(19) United States
(12) Patent Application Publication
(10) Pub. No.: US 2021/0315933 A1
(43) Pub. Date: Oct. 14, 2021

(54) COMPOSITIONS AND METHODS FOR TCR REPROGRAMMING USING TARGET SPECIFIC FUSION PROTEINS

(71) Applicant: TCR2 THERAPEUTICS INC., Cambridge, MA (US)

(72) Inventors: Patrick Alexander Baeuerle, Gauting (DE); Robert Hofmeister, Scituate, MA (US); Daniel Getts, Westminster, MA (US); Vania Ashminova, North Billerica, MA (US); Jian Ding, Newton, MA (US)

(21) Appl. No.: 17/261,823

(22) PCT Filed: Jul. 26, 2019
(86) PCT No.: PCT/US2019/043690

§ 371 Date: Jan. 20, 2021

Related U.S. Application Data
(60) Provisional application No. 62/703,824, filed on Jul. 26, 2018.
(60) Provisional application No. 62/703,834, filed on Jul. 26, 2018.
(60) Provisional application No. 62/725,066, filed on Aug. 30, 2018.
(60) Provisional application No. 62/727,459, filed on Sep. 5, 2018.
(60) Provisional application No. 62/727,469, filed on Sep. 5, 2018.

(51) Int. Cl. A61K 35/17; C07K 14/725; C07K 16/30; A61P 35/00; C07K 16/28

(52) U.S. Cl. CPC A61K 35/17; C07K 14/7051; C07K 16/3092; A61P 35/00; A61K 2039/505; C07K 2319/03; C07K 2319/30; C07K 2317/622; C07K 2317/569; C07K 16/2866

(57) ABSTRACT

Provided herein are T cell receptor (TCR) fusion proteins (TFPs), T cells engineered to express one or more MUC16 or IL 13Rα2 or MSLN TFPs, and methods of use thereof for the treatment of diseases, including cancer.

### CROSS-REFERENCE

This application claims the benefit of U.S. Provisional Patent Application No. 62/703,824, filed Jul. 26, 2018, U.S. Provisional Patent Application No. 62/725,066, filed Aug. 30, 2018, U.S. Provisional Patent Application No. 62/703,834, filed Jul. 26, 2018, U.S. Provisional Patent Application No. 62/727,469, filed Sep. 5, 2018, and U.S. Provisional Patent Application No. 62/727,459, filed Sep. 5, 2018, each of which is entirely incorporated herein by reference.

### BACKGROUND OF THE INVENTION

Most patients with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue, or are poorly immunogenic. Furthermore, cancer cells use multiple mechanisms to render themselves invisible or hostile to the initiation and propagation of an immune attack by cancer immunotherapies.

Recent developments using chimeric antigen receptor (CAR) modified autologous T cell therapy, which relies on redirecting genetically engineered T cells to a suitable cell-surface molecule on cancer cells, show promising results in harnessing the power of the immune system to treat B cell malignancies (see, e.g., Sadelain et al., Cancer Discovery 3:388-398 (2013)). The clinical results with CD-19-specific CAR-T cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma.

Besides the ability of genetically modified T cells expressing a CAR or a second TCR to recognize and destroy respective target cells in vitro/ex vivo, successful patient therapy with engineered T cells requires the T cells to be capable of strong activation, expansion, persistence over time, and, in case of relapsing disease, to enable a ‘memory’ response. High and manageable clinical efficacy of CAR-T cells is currently limited to BCMA- and CD-19-positive B cell malignancies and to NY-ESO-1-peptide expressing synovial sarcoma patients expressing HLA-A2. There is a clear need to improve genetically engineered T cells to more broadly act against various human malignancies.

### SUMMARY

Provided herein are T cell receptor (TCR) fusion proteins (TFPs), T cells engineered to express one or more TFPs, and methods of use thereof for the treatment of diseases.

According to an aspect, provided herein is a pharmaceutical composition comprising (I) a T cell from a human subject, wherein the T cell comprises a recombinant nucleic acid molecule encoding a T cell receptor (TCR) fusion protein (TFP) comprising (a) a TCR subunit comprising (i) at least a portion of a TCR extracellular domain, (ii) a TCR transmembrane domain, and (iii) a TCR intracellular domain comprising a stimulatory domain from an intracellular signaling domain; and (b) an antigen binding domain comprising an anti-MUC16 binding domain, an anti-IL13Rα2 binding domain or an anti-mesothelin (MSLN) binding domain; and (II) a pharmaceutically acceptable carrier; wherein the TCR subunit and the antigen binding domain are operatively linked; wherein the TFP functionally interacts with a TCR when expressed in the T cell.

In some embodiments, the T cell exhibits increased cytotoxicity to a cell expressing an antigen that specifically interacts with the antigen binding domain compared to a T cell not containing the TFP.

In some embodiments, the TCR extracellular domain, the TCR transmembrane domain, and the TCR intracellular domain of the TCR subunit are derived from a TCR alpha chain, a TCR beta chain, a TCR gamma chain, a TCR delta chain, CD3 epsilon, CD3 gamma, or CD3 delta.

In some embodiments, the TCR extracellular domain, the TCR transmembrane domain, and the TCR intracellular domain of the TCR subunit are derived from a single subunit of a TCR complex, wherein the single subunit is a TCR alpha chain, a TCR beta chain, a TCR gamma chain, a TCR delta chain, CD3 epsilon, CD3 gamma, or CD3 delta.

According to an aspect, provided herein is a pharmaceutical composition comprising (I) a T cell from a human subject, wherein the T cell comprises a recombinant nucleic acid molecule encoding a T cell receptor (TCR) fusion protein (TFP) comprising (a) a TCR subunit comprising (i) at least a portion of a TCR extracellular domain, (ii) a transmembrane domain, and (iii) a TCR intracellular domain comprising a stimulatory domain from an intracellular signaling domain; and (b) a scFv or single domain antibody comprising an anti-MUC16 binding domain, an anti-IL13Rα2 binding domain or an anti-mesothelin (MSLN) binding domain; and (II) a pharmaceutically acceptable carrier; wherein the TCR subunit and the anti-MUC16 or the anti-IL13Rα2 or the anti-MSLN binding domain are operatively linked; wherein the extracellular, transmembrane, and intracellular signaling domains of the TCR subunit are derived only from a TCR subunit other than a TCR alpha chain or a TCR beta chain; wherein the TFP functionally interacts with a TCR when expressed in the T cell; and wherein the T cell exhibits increased cytotoxicity to a cell expressing an antigen that specifically interacts with the anti-MUC16 or an anti-IL13Rα2 binding domain compared to a T cell not containing the TFP.

In some embodiments, the sequence encoding the anti-MUC16 or the anti-IL13Rα2 or the anti-MSLN binding domain is connected to the sequence encoding the TCR extracellular domain by a sequence encoding a linker. In some embodiments, the linker comprises (G₄S)ₙ, wherein G is glycine, S is serine, and n is an integer from 1 to 4.

In some embodiments, the anti-MUC16 binding domain comprises (a) a heavy chain (HC) CDR1 sequence GRTVSSLF, GRAVSSLF, or GDSLDGYV, (b) a HC CDR2 sequence ISRYSLYT, or ISGDGSMR, and (c) a HC CDR3 sequence ASKLEYTSNDYDS, or AADPPTWDY. In some embodiments, the anti-MUC16 binding domain comprises a sequence having at least 70%, at least 75%, at least 80%, at least 85%, at least 90%, at least 95%, or 100% sequence identity of SEQ ID NO:15, SEQ ID NO:20, SEQ ID NO:25, SEQ ID NO:30, SEQ ID NO:35, or SEQ ID NO:40.

In some embodiments, the anti-IL13Rα2 binding domain comprises (a) a heavy chain (HC) CDR1 sequence GFTSDYYI or GFASDDYI, (b) a HC CDR2 sequence ISSKYANT or ISSRYANT, and (c) a HC CDR3 sequence AADTRRYTCPDIATMHRNFDS or AMDSRRVTCPEISTMHRNFDS. In some embodiments, the anti-IL13Rα2 binding domain comprises a sequence having at least 70%, at least 75%, at least 80%, at least 85%, at least 90%, at least 95%, or 100% sequence identity of SEQ ID NO:51, SEQ ID NO:56, SEQ ID NO:61, SEQ ID NO:66, SEQ ID NO:71, or SEQ ID NO:76. In some embodiments, the sequence identity is determined using a BLAST algorithm with a word size of 6, a BLOSUM62 matrix, an existence penalty of 11 and an extension penalty of 1.

In some embodiments, the anti-MSLN binding domain comprises a sequence having at least 70%, at least 75%, at least 80%, at least 85%, at least 90%, at least 95%, or 100% sequence identity of SEQ ID NO:97 or SEQ ID NO:98. In some embodiments, the pharmaceutical composition is substantially free of serum. In some embodiments, the scFv or single domain antibody is a scFv. In some embodiments, the scFv or single domain antibody is a single domain antibody. In some embodiments, the single domain antibody is a V_{H} domain. In some embodiments, the encoded anti-TAA binding domain comprises an anti-TAA binding domain, and wherein the T cells have greater than or more efficient cytotoxic activity than CD8+ or CD4+ T cells comprising a nucleic acid encoding a chimeric antigen receptor (CAR) comprising (a) the anti-TAA binding domain, operatively linked to (b) at least a portion of a CD28 extracellular domain (c) a CD28 transmembrane domain (d) at least a portion of a CD28 intracellular domain and (e) a CD3 zeta intracellular domain. In some embodiments, the encoded TFP molecule functionally interacts with an endogenous TCR complex, at least one endogenous TCR polypeptide, or a combination thereof when expressed in the T cell. In some embodiments, the T cell is a primary T cell. In some embodiments, the T cell is a human CD4+ T cell. In some embodiments, the T cell is a human CD8+ T cell. In some embodiments, the T cell further comprises a nucleic acid encoding a first polypeptide comprising at least a portion of an inhibitory molecule selected from the group consisting of PD-1 and BTLA, wherein the at least a portion of an inhibitory molecule is associated with a second polypeptide comprising a positive signal from an intracellular signaling domain. In some embodiments, the second polypeptide comprises a costimulatory domain and primary signaling domain from a protein selected from the group consisting of CD28, CD27, ICOS, CD3ζ, 41-BB, OX40, GITR, CD30, CD40, ICOS, BAFFR, HVEM, LFA-1, CD2, CD7, LIGHT, NKG2C, SLAMF7, NKp80, CD160, and B7-H3. In some embodiments, production of IL-2 or IFNγ by the T cell is increased in the presence of a cell expressing an antigen that specifically interacts with the anti-TAA binding domain compared to a T cell not containing the TFP. In some embodiments, the cell is a population of human CD8+ or CD4+ T cells, wherein an individual T cell of the population comprises at least two TFP molecules, or at least two T cells of the population collectively comprise at least two TFP molecules; wherein the at least two TFP molecules comprise an anti-TAA binding domain, a TCR extracellular domain, a transmembrane domain, and an intracellular domain; and wherein at least one of the at least two TFP molecules functionally interacts with an endogenous TCR complex, at least one endogenous TCR polypeptide, or a combination thereof. In some embodiments, the TCR subunit is derived only from CD3 epsilon. In some embodiments, the TCR subunit is derived only from CD3 gamma. In some embodiments, the TCR subunit is derived only from CD3 delta.

According to an aspect, provided herein is a method of providing an anti-tumor immunity in a mammal comprising administering to the mammal an effective amount of a population of T cells transduced with a recombinant nucleic acid molecule encoding a T cell receptor (TCR) fusion protein (TFP) comprising (a) a TCR subunit comprising (i) at least a portion of a TCR extracellular domain, and (ii) a TCR intracellular domain comprising a stimulatory domain from a TCR intracellular signaling domain; and (b) an antibody domain comprising an antigen binding domain that is an anti-TAA binding domain; wherein the TCR subunit and the antibody domain are operatively linked, wherein the TFP incorporates into a TCR when expressed in a T cell, and wherein lower levels of cytokines are released following treatment compared to the cytokine levels of a mammal treated with a CAR-T cell comprising the same antibody domain. In some embodiments, the TCR intracellular signaling domain is derived from CD3 epsilon or CD3 gamma. In some embodiments, the TCR subunit further comprises a TCR transmembrane domain. In some embodiments, the TCR extracellular domain, the TCR transmembrane domain, and the TCR intracellular domain are derived from a TCR alpha chain, a TCR beta chain, a TCR delta chain, a TCR gamma chain, CD3 epsilon, CD3 gamma, or CD3 delta. In some embodiments, the TCR extracellular domain, the TCR transmembrane domain, and the TCR intracellular domain are derived from a single subunit of a TCR complex, wherein the single subunit is a TCR alpha chain, a TCR beta chain, a TCR delta chain, a TCR gamma chain, CD3 epsilon, CD3 gamma, or CD3 delta.

In some embodiments, the antibody domain is an anti-MUC16 V_{HH} domain having at least 70%, at least 75%, at least 80%, at least 85%, at least 90%, at least 95%, or 100% sequence identity to a sequence set forth in SEQ ID NO:15, SEQ ID NO:20, SEQ ID NO:25, SEQ ID NO:30, SEQ ID NO:35, or SEQ ID NO:40. In some embodiments, the antibody domain is an anti-IL13Rα2 V_{HH} domain having at least 70%, at least 75%, at least 80%, at least 85%, at least 90%, at least 95%, or 100% sequence identity to a sequence set forth in SEQ ID NO:51, SEQ ID NO:56, SEQ ID NO:61, SEQ ID NO:66, SEQ ID NO:71, or SEQ ID NO:76. In some embodiments, the sequence identity is determined using a BLAST algorithm with a word size of 6, a BLOSUM62 matrix, an existence penalty of 11 and an extension penalty of 1. In some embodiments, the cell is an autologous T cell. In some embodiments, the cell is an allogeneic T cell. In some embodiments, the mammal is a human.

According to an aspect, provided herein is a method of treating a mammal having a disease associated with expression of a tumor associated antigen (TAA) (e.g., MUC16, IL13Rα2, or MSLN) comprising administering to the mammal an effective amount of a population of T cells transduced with a recombinant nucleic acid molecule encoding a T cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of CD3epsilon or CD3gamma; and (b) an antibody domain comprising an antigen binding domain that is an anti-TAA binding domain; wherein the TCR subunit and the antibody domain are operatively linked, wherein the TFP incorporates into a TCR when expressed in a T cell, and wherein lower levels of cytokines are released following treatment compared to the cytokine levels of a mammal treated with a CAR-T cell comprising the same antibody domain.

In some embodiments, the antibody domain is a V_{HH} domain having at least 70%, at least 75%, at least 80%, at least 85%, at least 90%, at least 95%, or 100% sequence identity to a sequence set forth in SEQ ID NO:15, SEQ ID NO:20, SEQ ID NO:25, SEQ ID NO:30, SEQ ID NO:35, or SEQ ID NO:40. In some embodiments, the antibody domain is a V_{HH} domain having at least 70%, at least 75%, at least 80%, at least 85%, at least 90%, at least 95%, or 100% sequence identity to a sequence set forth in SEQ ID NO:51, SEQ ID NO:56, SEQ ID NO:61, SEQ ID NO:66, SEQ ID NO:71, or SEQ ID NO:76. In some embodiments, the sequence identity is determined using a BLAST algorithm with a word size of 6, a BLOSUM62 matrix, an existence penalty of 11 and an extension penalty of 1. In some embodiments, the cell is an autologous T cell. In some embodiments, the cell is an allogeneic T cell. In some embodiments, the disease associated with the TAA expression is selected from the group consisting of a proliferative disease, a cancer, a malignancy, and a non-cancer related indication associated with expression of the TAA, e.g., MUC16, IL13Rα2, or MSLN. In some embodiments, the disease is a cancer selected from the group consisting of glioblastoma, mesothelioma, renal cell carcinoma, stomach cancer, breast cancer, lung cancer, ovarian cancer, prostate cancer, colon cancer, cervical cancer, brain cancer, liver cancer, pancreatic cancer, thyroid cancer, bladder cancer, ureter cancer, kidney cancer, endometrial cancer, esophageal cancer, gastric cancer, thymic carcinoma, cholangiocarcinoma, stomach cancer, and any combination thereof. In some embodiments, the disease is a cancer selected from the group consisting of glioblastoma, mesothelioma, papillary serous ovarian adenocarcinoma, clear cell ovarian carcinoma, mixed Mullerian ovarian carcinoma, endometroid mucinous ovarian carcinoma, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with MUC16 expression, a disease associated with IL13Rα2 expression, a disease associated with MSLN expression, and any combination thereof. In some embodiments, the cells expressing a TFP molecule are administered in combination with an agent that increases the efficacy of a cell expressing a TFP molecule. In some embodiments, for a given cytokine, at least 10% less amount of the given cytokine is released following treatment compared to an amount of the given cytokine of a mammal treated with a CAR-T cell comprising the same antibody domain. In some embodiments, the given cytokine comprises one or more cytokines selected from the group consisting of IL-2, IFN-γ, IL-4, TNF-α, IL-6, IL-13, IL-5, IL-10, sCD137, GM-CSF, MIP-1α, MIP-1β, and any combination thereof. In some embodiments, a tumor growth in the mammal is inhibited such that a size of the tumor is at most 10%, at most 20%, at most 30%, at most 40%, at most 50%, or at most 60% of a size of a tumor in a mammal treated with T cells that do not express the TFP after at least 8 days of treatment, wherein the mammal treated with T cells expressing TFP and the mammal treated with T cells that do not express the TFP have the same tumor size before the treatment. In some embodiments, the tumor growth in the mammal is completely inhibited. In some embodiments, the tumor growth in the mammal is completely inhibited for at least 20 days, at least 30 days, at least 40 days, at least 50 days, at least 60 days, at least 70 days, at least 80 days, at least 90 days, at least 100 days, or more. In some embodiments, the population of T cells transduced with TFP kill similar amount of tumor cells compared to the CAR-T cells comprising the same antibody domain. In some embodiments, the population of T cells transduced with the TFP have a different gene expression profile than the CAR-T cells comprising the same antibody domain. In some embodiments, an expression level of a gene is different in the T cells transduced with the TFP than an expression level of the gene in the CAR-T cells comprising the same antibody domain. In some embodiments, the gene has a function in antigen presentation, TCR signaling, homeostasis, metabolism, chemokine signaling, cytokine signaling, toll like receptor signaling, MMP and adhesion molecule signaling, or TNFR related signaling.

According to an aspect, provided herein is a recombinant nucleic acid molecule encoding a T-cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of CD3 epsilon; and (b) an antibody domain comprising an anti-TAA binding domain; wherein the TCR subunit and the antibody domain are operatively linked, and wherein the TFP incorporates into a TCR when expressed in a T-cell.

According to an aspect, provided herein is an recombinant nucleic acid molecule encoding a T-cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of CD3 gamma; and (b) an antibody domain comprising an anti-TAA binding domain; wherein the TCR subunit and the antibody domain are operatively linked, and wherein the TFP incorporates into a TCR when expressed in a T-cell.

According to an aspect, provided herein is a recombinant nucleic acid molecule encoding a T-cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of CD3 delta; and (b) an antibody domain comprising an anti-TAA binding domain; wherein the TCR subunit and the antibody domain are operatively linked, and wherein the TFP incorporates into a TCR when expressed in a T-cell.

According to an aspect, provided herein is a recombinant nucleic acid molecule encoding a T-cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of TCR alpha; and (b) an antibody domain comprising an anti-TAA binding domain; wherein the TCR subunit and the antibody domain are operatively linked, and wherein the TFP incorporates into a TCR when expressed in a T-cell.

According to an aspect, provided herein is a recombinant nucleic acid molecule encoding a T-cell receptor (TCR) fusion protein (TFP) comprising (a) a TCR subunit comprising (i) at least a portion of a TCR extracellular domain, and (ii) a TCR intracellular domain comprising a stimulatory domain from an intracellular signaling domain of TCR beta; and (b) an antibody domain comprising an anti-TAA binding domain; wherein the TCR subunit and the antibody domain are operatively linked, and wherein the TFP incorporates into a TCR when expressed in a T-cell.

In some embodiments, the antibody domain is a human or humanized antibody domain. In some embodiments, the encoded antigen binding domain is connected to the TCR extracellular domain by a linker sequence. In some embodiments, the encoded linker sequence comprises (G₄S)ₙ, wherein n=1 to 4. In some embodiments, the TCR subunit comprises a TCR extracellular domain. In some embodiments, the TCR subunit comprises a TCR transmembrane domain. In some embodiments, the TCR subunit comprises a TCR intracellular domain. In some embodiments, the TCR subunit comprises (i) a TCR extracellular domain, (ii) a TCR transmembrane domain, and (iii) a TCR intracellular domain, wherein at least two of (i), (ii), and (iii) are from the same TCR subunit. In some embodiments, the TCR subunit comprises a TCR intracellular domain comprising a stimulatory domain selected from an intracellular signaling domain of CD3 epsilon, CD3 gamma or CD3 delta, or an amino acid sequence having at least one modification thereto. In some embodiments, the TCR subunit comprises an intracellular domain comprising a stimulatory domain selected from a functional signaling domain of 4-1BB and/or a functional signaling domain of CD3 zeta, or an amino acid sequence having at least one modification thereto. In some embodiments, the antibody domain comprises an antibody fragment. In some embodiments, the antibody domain comprises a scFv or a V_{H} domain.

In some embodiments, the recombinant nucleic acid molecule encodes (a) a heavy chain (HC) CDR1 sequence GRTVSSLF, GRAVSSLF, or GDSLDGYV, (b) a HC CDR2 sequence ISRYSLYT, or ISGDGSMR, and (c) a HC CDR3 sequence ASKLEYTSNDYDS, or AADPPTWDY. In some embodiments, the recombinant nucleic acid molecule encodes (a) a heavy chain (HC) CDR1 sequence GFTSDYYI or GFASDDYI, (b) a HC CDR2 sequence ISSKYANT or ISSRYANT, and (c) a HC CDR3 sequence AADTRRYTCPDIATMHRNFDS or AMDSRRVTCPEISTMHRNFDS. In some embodiments, the isolated nucleic acid molecule encodes a heavy chain variable domain having at least 70%, at least 75%, at least 80%, at least 85%, at least 90%, at least 95%, or 100% sequence identity to a sequence set forth in SEQ ID NO:15, SEQ ID NO:20, SEQ ID NO:25, SEQ ID NO:30, SEQ ID NO:35, or SEQ ID NO:40. In some embodiments, the antibody domain is a V_{HH} domain having at least 70%, at least 75%, at least 80%, at least 85%, at least 90%, at least 95%, or 100% sequence identity to a sequence set forth in SEQ ID NO:51, SEQ ID NO:56, SEQ ID NO:61, SEQ ID NO:66, SEQ ID NO:71, or SEQ ID NO:76. In some embodiments, the sequence identity is determined using a BLAST algorithm with a word size of 6, a BLOSUM62 matrix, an existence penalty of 11 and an extension penalty of 1. In some embodiments, the TFP includes an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the encoded TFP includes a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the encoded TFP includes a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the recombinant nucleic acid molecule further comprises a sequence encoding a costimulatory domain. In some embodiments, the costimulatory domain is a functional signaling domain obtained from a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In some embodiments, the at least one but not more than 20 modifications thereto comprise a modification of an amino acid that mediates cell signaling or a modification of an amino acid that is phosphorylated in response to a ligand binding to the TFP. In some embodiments, the isolated nucleic acid molecule is mRNA. In some embodiments, the TFP includes an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the ITAM replaces an ITAM of CD3 gamma, CD3 delta, or CD3 epsilon. In some embodiments, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit. In some embodiments, the nucleic acid comprises a nucleotide analog. In some embodiments, the nucleotide analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some embodiments, the recombinant nucleic acid molecule further comprises a leader sequence.

According to an aspect, provided herein is a recombinant polypeptide molecule encoded by the recombinant nucleic acid molecule described herein.

According to an aspect, provided herein is a recombinant TFP molecule comprising an anti-TAA binding domain (e.g., a MUC16, IL13Ra2, or MSLN binding domain), a TCR extracellular domain, a transmembrane domain, and an intracellular domain.

According to an aspect, provided herein is a recombinant TFP molecule comprising an anti-TAA binding domain, a TCR extracellular domain, a transmembrane domain, and an intracellular signaling domain, wherein the TFP molecule is capable of functionally interacting with an endogenous TCR complex and/or at least one endogenous TCR polypeptide.

According to an aspect, provided herein is a recombinant TFP molecule comprising an anti-TAA binding domain, a TCR extracellular domain, a transmembrane domain, and an intracellular signaling domain, wherein the TFP molecule is capable of functionally integrating into an endogenous TCR complex.

In some embodiments, the recombinant TFP molecule comprises an antibody or antibody fragment comprising an anti-MUC16, an anti-IL13Rα2, or an anti-MSLN binding domain, a TCR extracellular domain, a transmembrane domain, and an intracellular domain.

In some embodiments, the anti-TAA binding domain is a scFv, a V_{HH} or a V_{H} domain.

In some embodiments, the anti-TAA binding domain comprises a heavy chain with 95-100% identity to an amino acid sequence of SEQ ID NO:15, SEQ ID NO:20, SEQ ID NO:25, SEQ ID NO:30, SEQ ID NO:35, or SEQ ID NO:40, a functional fragment thereof, or an amino acid sequence thereof having at least one but not more than 30 modifications. In some embodiments, the anti-TAA binding domain comprises a heavy chain with 95-100% identity to an amino acid sequence of NO:51, SEQ ID NO:56, SEQ ID NO:61, SEQ ID NO:66, SEQ ID NO:71, or SEQ ID NO:76, a functional fragment thereof, or an amino acid sequence thereof having at least one but not more than 30 modifications.

In some embodiments, the sequence identity is determined using a BLAST algorithm with a word size of 6, a BLOSUM62 matrix, an existence penalty of 11 and an extension penalty of 1.

In some embodiments, the recombinant TFP molecule comprises a TCR extracellular domain that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the anti-TAA binding domain is connected to the TCR extracellular domain by a linker sequence. In some embodiments, the linker region comprises (G₄S)ₙ, wherein n=1 to 4.

According to an aspect, provided herein is a nucleic acid comprising a sequence encoding a TFP.

In some embodiments, the nucleic acid is selected from the group consisting of a DNA and a RNA. In some embodiments, the nucleic acid is a mRNA. In some embodiments, the nucleic acid comprises a nucleotide analog. In some embodiments, the nucleotide analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some embodiments, the nucleic acid further comprises a promoter. In some embodiments, the nucleic acid is an in vitro transcribed nucleic acid. In some embodiments, the nucleic acid further comprises a sequence encoding a poly(A) tail. In some embodiments, the nucleic acid further comprises a 3′UTR sequence.

According to an aspect, provided herein is a vector comprising a nucleic acid molecule encoding a TFP.

In some embodiments, the vector is selected from the group consisting of a DNA, a RNA, a plasmid, a lentivirus vector, adenoviral vector, a Rous sarcoma viral (RSV) vector, or a retrovirus vector. In some embodiments, the vector further comprises a promoter. In some embodiments, the vector is an in vitro transcribed vector. In some embodiments, a nucleic acid sequence in the vector further comprises a poly(A) tail. In some embodiments, a nucleic acid sequence in the vector further comprises a 3′UTR.

In some embodiments, provided herein is a cell comprising the recombinant nucleic acid molecule described herein. In some embodiments, provided herein is a polypeptide molecule. In some embodiments, provided herein is a TFP molecule. In some embodiments, provided herein is a nucleic acid. In some embodiments, provided herein is a vector. In some embodiments, the cell is a human T cell. In some embodiments, the T cell is a CD8+ or CD4+ T-cell or CD4+CD8+ T cell. In some embodiments, the T cell is a gamma delta T cell. In some embodiments, the cell further comprises a nucleic acid encoding an inhibitory molecule that comprises a first polypeptide that comprises at least a portion of an inhibitory molecule, associated with a second polypeptide that comprises a positive signal from an intracellular signaling domain. In some embodiments, the inhibitory molecule comprise first polypeptide that comprises at least a portion of PD1 and a second polypeptide comprising a costimulatory domain and primary signaling domain.

According to an aspect, provided herein is a human CD8+ or CD4+ T-cell comprising at least two TFP molecules, the TFP molecules comprising an anti-TAA binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T-cell.

According to an aspect, provided herein is a protein complex comprising: (a) a TFP molecule comprising an anti-TAA binding domain, a TCR extracellular domain, a transmembrane domain, and an intracellular domain; and (b) at least one endogenous TCR subunit or endogenous TCR complex.

In some embodiments, the TCR comprises an extracellular domain or portion thereof of a protein selected from the group consisting of TCR alpha chain, a TCR beta chain, a CD3 epsilon TCR subunit, a CD3 gamma TCR subunit, and a CD3 delta TCR subunit. In some embodiments, the anti-TAA binding domain is connected to the TCR extracellular domain by a linker sequence. In some embodiments, the linker region comprises (G₄S)ₙ, wherein n=1 to 4.

According to an aspect, provided herein is a protein complex comprising (a) a TFP encoded by any of the recombinant nucleic acid molecules disclosed herein, and (b) at least one endogenous TCR subunit or endogenous TCR complex.

According to an aspect, provided herein is a protein complex comprising: (a) a TFP molecule comprising an anti-TAA binding domain, a TCR extracellular domain, a transmembrane domain, and an intracellular domain; and (b) at least one endogenous TCR subunit or endogenous TCR complex.

In some embodiments, the TCR comprises an extracellular domain or portion thereof of a protein selected from the group consisting of TCR alpha chain, a TCR beta chain, a CD3 epsilon TCR subunit, a CD3 gamma TCR subunit, and a CD3 delta TCR subunit. In some embodiments, the anti-TAA binding domain is connected to the TCR extracellular domain by a linker sequence. In some embodiments, the linker region comprises (G₄S)ₙ, wherein n=1 to 4.

According to an aspect, provided herein is a human CD8+ or CD4+ T-cell comprising at least two different TFP proteins per the protein complex.

According to an aspect, provided herein is a human CD8+ or CD4+ T-cell comprising at least two different TFP molecules encoded by the isolated nucleic acid molecules described herein.

According to an aspect, provided herein is a population of human CD8+ or CD4+ T-cells, wherein the T-cells of the population individually or collectively comprise at least two TFP molecules, the TFP molecules comprising an anti-TAA binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T-cell.

According to an aspect, provided herein is a population of human CD8+ or CD4+ T-cells, wherein the T-cells of the population individually or collectively comprise at least two TFP molecules encoded by the recombinant nucleic acid molecule described herein.

According to an aspect, provided herein is a method of making a cell comprising transducing a T-cell with the recombinant nucleic acid molecule described herein, the nucleic acid described herein, or the vector described herein.

According to an aspect, provided herein is a method of generating a population of RNA-engineered cells comprising introducing an in vitro transcribed RNA or synthetic RNA into a cell, where the RNA comprises a nucleic acid encoding the TFP molecule described herein.

According to an aspect, provided herein is a method of providing an anti-tumor immunity in a mammal comprising administering to the mammal an effective amount of the recombinant nucleic acid molecule described herein, the polypeptide molecule described herein, a cell expressing the polypeptide molecule described herein, the TFP molecule described herein, the nucleic acid described herein, the vector described herein, or the cell described herein. In some embodiments, the cell is an autologous T-cell. In some embodiments, the cell is an allogeneic T-cell. In some embodiments, the mammal is a human.

According to an aspect, provided herein is a method of treating a mammal having a disease associated with expression of MUC16, IL13Rα2, or MSLN comprising administering to the mammal an effective amount of the isolated nucleic acid molecule, the polypeptide molecule described herein, a cell expressing the polypeptide molecule, the TFP molecule described herein, the nucleic acid, the vector, or the cell described herein. In some embodiments, the disease associated with MUC16, IL13Rα2, or MSLN expression is selected from the group consisting of a proliferative disease, a cancer, a malignancy, myelodysplasia, a myelodysplastic syndrome, a preleukemia, a non-cancer related indication associated with expression of MUC16, IL13Rα2, or MSLN. In some embodiments, the disease is pancreatic cancer, ovarian cancer, breast cancer, or any combination thereof. In some embodiments, the cells expressing a TFP molecule are administered in combination with an agent that increases the efficacy of a cell expressing a TFP molecule. In some embodiments, less cytokines are released in the mammal compared a mammal administered an effective amount of a T-cell expressing an anti-TAA chimeric antigen receptor (CAR). In some embodiments, the cells expressing a TFP molecule are administered in combination with an agent that ameliorates one or more side effects associated with administration of a cell expressing a TFP molecule. In some embodiments, the cells expressing a TFP molecule are administered in combination with an agent that treats the disease associated with the TAA, e.g., MUC16, IL13Rα2, or MSLN. In some embodiments, the polypeptide molecule, a cell expressing the polypeptide molecule, the recombinant TFP, the nucleic acid, the vector, the complex, or the cell, for use as a medicament.

According to an aspect, provided herein is a recombinant nucleic acid molecule encoding a TFP, a polypeptide molecule of a TFP, a cell expressing the polypeptide molecule of a TFP, a recombinant TFP, a nucleic acid encoding a TFP, a vector comprising a nucleic acid encoding a TFP, a protein complex, or a cell, for use as a medicament. According to an aspect, provided herein is a method of treating a mammal having a disease associated with expression of MUC16, IL13Rα2, or MSLN comprising administering to the mammal an effective amount of the recombinant nucleic acid molecule, the polypeptide molecule, a cell expressing the polypeptide molecule, the recombinant TFP molecule, the nucleic acid, the vector, or the cell, wherein less cytokines are released in the mammal compared a mammal administered an effective amount of a T-cell expressing an anti-TAA chimeric antigen receptor (CAR).

According to an aspect, provided herein is a pharmaceutical composition comprising (I) a T cell from a human subject, wherein the T cell comprises a recombinant nucleic acid molecule encoding a T cell receptor (TCR) fusion protein (TFP) comprising (a) a TCR subunit comprising (i) at least a portion of a TCR extracellular domain, (ii) a TCR transmembrane domain, and (iii) a TCR intracellular domain comprising a stimulatory domain from an intracellular signaling domain; and (b) an antigen binding domain comprising an anti-IL13Rα2 binding domain; and (II) a pharmaceutically acceptable carrier; wherein the TCR subunit and the anti-IL13Rα2 binding domain are operatively linked; wherein the TFP functionally interacts with a TCR when expressed in the T cell. In some embodiments, the TCR extracellular, the TCR transmembrane domain, and the TCR intracellular domain of the TCR subunit are derived from a TCR alpha chain, a TCR beta chain, a TCR gamma chain, a TCR delta chain, CD3 epsilon, CD3 delta, or CD3 gamma. In some embodiments, the TCR extracellular domain, the TCR transmembrane domain, and the TCR intracellular domain of the TCR subunit are derived from a single subunit of a TCR complex, wherein the single subunit is a TCR alpha chain, a TCR beta chain, a TCR gamma chain, a TCR delta chain, CD3 epsilon, CD3 gamma, or CD3 delta. In some embodiments, the T cell exhibits increased cytotoxicity to a cell expressing an antigen that specifically interacts with the anti-IL13Rα2 binding domain compared to a T cell not containing the TFP.

According to an aspect, provided herein is a method of providing an anti-tumor immunity in a mammal comprising administering to the mammal an effective amount of a population of T cells transduced with a recombinant nucleic acid molecule encoding a T-cell receptor (TCR) fusion protein (TFP) comprising a TCR subunit comprising at least a portion of a TCR extracellular domain, and a TCR intracellular domain comprising a stimulatory domain from an intracellular signaling domain; and a human or humanized antibody domain comprising an antigen binding domain that is an anti-mesothelin binding domain; wherein the TCR subunit and the antibody domain are operatively linked, wherein the TFP incorporates into a TCR when expressed in a T-cell, and wherein the population of T cells preferentially kill tumor cells with higher mesothelin expression comparted with tumor cells with lower mesothelin expression.

In some embodiments, the TCR subunit further comprises a TCR transmembrane domain. In some embodiments, the TCR extracellular domain, the TCR transmembrane domain, and the TCR intracellular domain are derived from a TCR alpha chain, a TCR beta chain, a TCR gamma chain, a TCR delta chain, CD3 epsilon, CD3 gamma, or CD3 delta. In some embodiments, the TCR extracellular domain, the TCR transmembrane domain, and the TCR intracellular domain of the TCR subunit are derived from a single subunit of a TCR complex, wherein the single subunit is a TCR alpha chain, a TCR beta chain, a TCR gamma chain, a TCR delta chain, CD3 epsilon, CD3 gamma, or CD3 delta. In some embodiments, the TCR intracellular signaling domain is derived from CD3 epsilon or CD3 gamma.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts example IL13Rα2 clone sequences.

FIG. 2 is a diagram illustrating the way the Pall Forte Bio Dip & Read AHC epitope binning assay was carried out. The AHC biosensor tip was coupled to the 4H111 scFv-Fc antibody (4H111) via the Fc domain which was then bound to the antigen peptide (“Ag”, e.g., MUC16, IL13Rα2, MSLN) via its Fv domain. The sdAbs (Ab2) were then added at 100 nM each to assess competition for binding to the antigen with the 4H111 scFv-Fc antibody.

FIG. 3A depicts data from a tumor cell lysis assay testing the in vitro activity of anti-IL13Rα2 nanobodies.

FIG. 3B depicts experimental data showing the ability of TFP T cells to induce IFNγ and IL-2 production.

FIG. 3C depicts experimental data from a tumor cell lysis assay testing the in vitro activity of anti-IL13Rα2 nanobodies.

FIG. 3D depicts experimental data showing that ability of TFP T cells to induce IFNγ and IL-2 production.

FIG. 3E depicts experimental data from a tumor cell lysis assay testing the in vitro activity of anti-IL13Rα2 nanobodies.

FIG. 3F depicts experimental data showing that ability of TFP T cells to induce IFNγ and IL-2 production.

FIG. 4 depicts experimental data from an IL13Rα2 U251 GBM model testing efficacy of IL13Rα2-TFP T cells. The graph shows the average tumor volumes measured by caliper over time after subcutaneous injection of 5×10⁶ U251 cells into NSG mice followed by intravenous administration of 1×10⁷ IL13Rα2-TFP T cells 4 days later.

FIGS. 5A-C show titration and measurement of binding affinity of parental (llama) and humanized anti-MUC16 single chain antibody (V_{HH}). FIG. 5A is a diagram illustrating the experimental procedure by which the V_{HH} binders produced in Example 5 are screened using an NTA biosensor (nickel coated surface). The His-tagged MUC16 sdAbs (3.25 μg/ml) are bound to the biosensor, and the MUC16 peptide is added at concentrations of 0, 1.56, 6.25, 25, 50, 100 or 200 nM. Buffer: 1× Octet; 1× Corning® Cellgro® PBS (cat. 21-040-CM) containing 0.02% Tween® 20 at 30° C. Sensors: Pall Forte Bio Dip & Read (cat. 18-5102). FIG. 5B (clone R3Mu4 parental and humanized variants) and FIG. 5C (clone R3Mu29 parental and humanized variants) show binding kinetics of the sdAbs to the MUC16 target (see also Table 1). These curves were used to derive binding affinity constant for each protein and to assess the effect of humanization on antigen binding.

FIGS. 6A-C show epitope binning of the anti-MUC16 sdAbs in comparison with the MUC-16 specific scFv-Fc tool binder 4H11 used as a positive control. FIG. 6A is a diagram illustrating the way the Pall Forte Bio Dip & Read AHC epitope binning assay was carried out as shown in FIG. 2 for IL13Rα2 binders. The AHC biosensor tip was coupled to the 4H11 scFv-Fc antibody (4H11) via the Fc domain which was then bound to the MUC16 antigen peptide (Ag) via its Fv domain. The sdAbs (Ab2) were then added at 100 nM each to assess competition for binding to the MUC16 antigen with the 4H11 scFv-Fc antibody. As shown in FIG. 6B, the MUC16 sdAbs—parental (llama) R3Mu4 and parental (llama) R3Mu29 show binding to the MUC16 peptide after 4H11 tool binder had already bound to it, demonstrating that the parental sdAbs recognize and bin to a different epitope of MUC16 peptide as compared to 4H11 scFv-Fc tool binder. The negative control with no antigen (MUC16 peptide) shows no binding, ruling out any chances of non-specific binding. FIG. 6C depicts epitopes of relevant antibodies in the context of the MUC16 ectodomain sequence.

FIG. 7 shows graphs of dose-dependent lysis of MUC16-ectodomain (“MUC16^{ecto}”) expressing cells by T cells expressing a T cell Receptor (TCR) fusion protein (TFP) that comprise a TCR subunit and an antibody domain comprising an anti-MUC16 binding domain. The T cells specifically killed SKOV3-MUC16Cterm ovarian cancer cells that were transduced to overexpress a C-terminal cell associated MUC16 form in a dose dependent manner, while the parental SKOV3 MUC16-negative cells were spared from T cell mediated killing. Likewise, T cells expressing the MUC16-TFP eliminated OVCAR3-MUC16-Cterm cells that overexpressed the cell-associated form of MUC16. Parental OVCAR3 cells expressing low levels of MUC16 were only killed at the highest TFP-T cell-to-target cell ratio. Likewise, TFP-T cells only released cytokines when MUC16 was present on the target cells, which supports the specificity of the single-domain antibody.

FIG. 8 depicts example experimental data showing the potency of MUC16-TFP in cellular assays using ovarian cell lines expressing high and low levels of MUC16. In these studies, MUC16-TFP was observed to have preferential killing abilities depending on the level of MUC16 on the tumor cell surface. More precisely, MUC16-TFP was observed to kill high MUC16 expressing tumor cells in a dose dependent fashion, whereas MUC16-TFP killing of low MUC16 expressing cells was not observed at the dose levels used in these assays.

FIGS. 9A-B depicts results of flow-cytometry-based MUC16^{ecto} copy number quantitation. 4H11-PE antibody-stained tumor cells were run on Fortessa® X-20 together with the Quantibrite beads. The geometric median fluorescent intensity (gMFI) was calculated for the cells as well as the beads (FIG. 9A). The beads stock contains 4 populations manufactured to have different number of PE molecules per bead (high, moderate, low, negative). A standard curve was generated based on the given copies of PE molecules per bead versus the measured MFI for each set of beads. The copy number of MUC16^{ecto} on tumor cells were then estimated based on the beads-generated standard curve. The copies of MUC16^{ecto} on OVCAR3, OVCAR3-MUC16^{ecto}, SKOV3 and SKOV3-MUC16^{ecto} cells were determined as 726, 3616, 39 and 2351, respectively (FIG. 9B).

FIGS. 10A-D show a series of graphs showing MUC16^{ecto} specific tumor cell lysis by MUC16 TFP-T cells. T cells expressing MUC16 TFPs specifically killed SKOV3-MUC16^{ecto} cells that overexpressed the cell-associated form of MUC16 (FIG. 10A), while the parental SKOV3 cells were not killed by T cells expressing MUC16 TFPs (FIG. 10B). Likewise, T cells expressing MUC16 TFPs eliminated OVCAR3-MUC16^{ecto} cells that overexpressed the cell-associated form of MUC16 (FIG. 0C). Parental OVCAR3 cells expressing low levels of MUC16^{ecto} were only killed partially (FIG. 10D).

FIGS. 11A-H show a series of graphs showing MUC16^{ecto} specific cytokine production by MUC16 TFP-T cells. T cells expressing MUC16 TFPs secreted IFN-γ and IL-2 when co-cultured with SKOV3-MUC16^{ecto} cells (FIGS. 11A and 11E, respectively) or OVCAR3-MUC16^{ecto} cells (FIGS. 11C and 11G, respectively), but not with SKOV3 cells (FIGS. 11B and 11F, respectively) or OVCAR3 cells (FIGS. 11D and 11H, respectively).

FIG. 12 depicts MUC16^{ecto} specific proliferation of T cells expressing MUC16-TFPs. MUC16^{ecto} specific proliferation of MUC16-TFP T cells were determined by monitoring the dilution of T cell tracing signal (decrease in signal intensity of CellTrace™) by flowcytometry analysis. T cells expressing MUC16-TFPs were labelled with CellTrace™ Far Red Proliferation Kit (Cat. #C34564ThermoFisher), then co-cultured with SKOV3 or SKOV3-MUC16^{ecto} cells at 1-to-1 ratio for 3 days. T cells expressing MUC16-TFPs labelled with CellTrace Far Red Proliferation kit were also stimulated with medium alone or with 1 μg/mL plate-bound anti-CD3 antibody (clone OKT-3, Cat #14-0037-82, Invitrogen) for 3 days. T cells expressing MUC16-TFPs showed MUC16^{ecto}-specific proliferation, demonstrated by the decrease of CellTrace signal when co-cultured with SKOV3-MUC16^{ecto} cells, but not SKOV3 cells (FIG. 12).

FIG. 13A-C depict a series of graphs showing in vivo activity of MUC16-TFP T cells. T cells expressing MUC16-TFPs were evaluated in NSG mouse xenograft models of human ovarian carcinoma cell lines, SKOV3-MUC16^{ecto} cells and OVCAR3-MUC16^{ecto} cells. In intraperitoneal model of SKOV3-MUC16^{ecto} cells, MUC16 TFP 1 showed significant decrease of the tumor burden in comparison to the baseline level on day 0 (day of T cell injection) (FIG. 13A). Consistently, MUC16 TFP1 significantly delayed the tumor growth in subcutaneous models of SKOV3-MUC16^{ecto} cells, when compared to NT T cells (FIG. 13B). In the intraperitoneal model of OVCAR3-MUC16^{ecto} cells, MUC16 TFP1 and MUC16 TFP2 both completed cleared tumor from the mice (FIG. 13C).

FIG. 14A-B is two graphs showing the differential killing ability of MSLN-TFP T cells against MSLN high (MSTO-MSLNhigh, 11006 copies of surface MSLN) and MSLN low tumors (MSTO-MSLNlow, 198 copies surface MSLN) in NSG mouse bearing either MSTO-MSLNhigh or MSTO-MSLNlow tumors. Tumor bearing mice were injected intravenously with non-transduced T cells (NT, 1×10⁷ total T cells) or MSLN-TFP T cells (1×10⁷ total T cells). MSLN-TFP T cells dramatically controlled the growth of MSLN high tumors, compared to NT T cells treated mice (FIG. 14A). On the other hand, limited anti-tumor response were observed in MSLN-TFP T cells treated mice with MSLN low tumors (FIG. 14B).

### DETAILED DESCRIPTION

Described herein are novel fusion proteins of TCR subunits, including CD3 epsilon, CD3 gamma and CD3 delta, and of TCR alpha and TCR beta chains with binding domains specific for cell surface antigens that have the potential to overcome limitations of existing approaches.

Described herein are novel fusion proteins that more efficiently kill target cells than CARs, but release comparable or lower levels of pro-inflammatory cytokines. These fusion proteins and methods of their use represent an advantage for T cell receptor (TCR) fusion proteins (TFPs) relative to CARs because elevated levels of these cytokines have been associated with dose-limiting toxicities for adoptive CAR-T therapies.

In one aspect, described herein are isolated nucleic acid molecules encoding a T cell Receptor (TCR) fusion protein (TFP) that comprise a TCR subunit and an antibody domain comprising an anti-tumor associated antigen (TAA) binding domain (e.g., an IL13Ra2, MUC16, or MSLN binding domain). In some embodiments, the antibody domain is a human or humanized antibody domain. In some embodiments, the TCR subunit comprises a TCR extracellular domain. In other embodiments, the TCR subunit comprises a TCR transmembrane domain. In yet other embodiments, the TCR subunit comprises a TCR intracellular domain. In further embodiments, the TCR subunit comprises (i) a TCR extracellular domain, (ii) a TCR transmembrane domain, and (iii) a TCR intracellular domain, wherein at least two of (i), (ii), and (iii) are from the same TCR subunit. In yet further embodiments, the TCR subunit comprises a TCR intracellular domain comprising a stimulatory domain selected from an intracellular signaling domain of CD3 epsilon, CD3 gamma or CD3 delta, or an amino acid sequence having at least one, two or three modifications thereto. In yet further embodiments, the TCR subunit comprises an intracellular domain comprising a stimulatory domain selected from a functional signaling domain of 4-1BB and/or a functional signaling domain of CD3 zeta, or an amino acid sequence having at least one, two or three modifications thereto.

In some embodiments, the isolated nucleic acid molecules comprise (i) a light chain (LC) CDR1, LC CDR2 and LC CDR3 of any anti-TAA light chain binding domain amino acid sequence provided herein, and/or (ii) a heavy chain (HC) CDR1, HC CDR2 and HC CDR3 of any anti-TAA heavy chain binding domain amino acid sequence provided herein.

In some embodiments, the isolated nucleic acid molecule comprise a HC CDR1, HC CDR2, and HC CDR3 of any anti-TAA heavy chain antibody or single domain antibody provided herein. For example, heavy chain antibodies or single domain antibodies can be found in the animals of the Camelidae family. The Camelidae family (camels: one-humped Camelus dromedaries and two-humped Camelus bactrianus; llamas: Lama glama, Lama guanicoe, Lama vicugna; alpaca: Vicugna pacos), of suborder Tylopoda, of order Artiodactyla have a special type of antibody in addition to classical antibodies in their serum. These antibodies, called heavy chain antibodies (HCAbs), are unique in their absence of the entire light chain and the first heavy chain constant region (C_{H}1). Antibodies similar to camelid heavy-chain only antibodies (cAbs) have also been found in wobbegong, nurse sharks and spotted ratfish.

In some embodiments, the light chain variable region comprises an amino acid sequence having at least one, two or three modifications but not more than 30, 20 or 10 modifications of an amino acid sequence of a light chain variable region provided herein, or a sequence with 95-99% identity to an amino acid sequence provided herein. In other embodiments, the heavy chain variable region comprises an amino acid sequence having at least one, two or three modifications but not more than 30, 20 or 10 modifications of an amino acid sequence of a heavy chain variable region provided herein, or a sequence with 95-99% identity to an amino acid sequence provided herein.

In some embodiments, the TFP includes an extracellular domain of a TCR subunit that comprises an extracellular domain or portion thereof of a protein selected from the group consisting of the alpha or beta chain of the T cell receptor, CD3 delta, CD3 epsilon, or CD3 gamma, or a functional fragment thereof, or an amino acid sequence having at least one, two or three modifications but not more than 20, 10 or 5 modifications thereto. In other embodiments, the encoded TFP includes a transmembrane domain that comprises a transmembrane domain of a protein selected from the group consisting of the alpha, beta chain of the TCR or TCR subunits CD3 epsilon, CD3 gamma and CD3 delta, or a functional fragment thereof, or an amino acid sequence having at least one, two or three modifications but not more than 20, 10 or 5 modifications thereto.

In some embodiments, the encoded TFP includes a transmembrane domain that comprises a transmembrane domain of a protein selected from the group consisting of the alpha, beta or zeta chain of the TCR, or CD3 epsilon, CD3 gamma, CD3 delta, CD45, CD2, CD4, CD5, CD8, CD9, CD16, CD22, CD33, CD28, CD37, CD64, CD80, CD86, CD134, CD137, CD154, and a functional fragment thereof. In some embodiments, the encoded TFP comprises a transmembrane domain of a protein comprising an amino acid sequence having at least one, two or three modifications but not more than 20, 10 or 5 modifications thereto, wherein the protein is selected from the group consisting of the alpha, beta or zeta chain of the TCR, or CD3 epsilon, CD3 gamma, CD3 delta, CD45, CD2, CD4, CD5, CD8, CD9, CD16, CD22, CD33, CD28, CD37, CD64, CD80, CD86, CD134, CD137, CD154, and a functional fragment thereof.

In some embodiments, the encoded anti-TAA binding domain is connected to the TCR extracellular domain by a linker sequence. In some instances, the encoded linker sequence comprises (G₄S)ₙ, wherein n=1 to 4. In some instances, the encoded linker sequence comprises a long linker (LL) sequence. In some instances, the encoded long linker sequence comprises (G₄S)ₙ, wherein n=2 to 4. In some instances, the encoded linker sequence comprises a short linker (SL) sequence. In some instances, the encoded short linker sequence comprises (G₄S)ₙ, wherein n=1 to 3.

In some embodiments, the isolated nucleic acid molecules further comprise a sequence encoding a costimulatory domain. In some instances, the costimulatory domain is a functional signaling domain obtained from a protein selected from the group consisting of DAP10, DAP12, CD30, LIGHT, OX40, CD2, CD27, CD28, CDS, ICAM-1, LFA-1 (CD11a/CD18), ICOS (CD278), and 4-1BB (CD137), or an amino acid sequence having at least one, two or three modifications but not more than 20, 10 or 5 modifications thereto.

In some embodiments, the isolated nucleic acid molecules further comprise a leader sequence.

Also provided herein are isolated polypeptide molecules encoded by any of the previously described nucleic acid molecules.

Also provided herein in another aspect, are isolated T cell receptor fusion protein (TFP) molecules that comprise an anti-TAA binding domain, a TCR extracellular domain, a transmembrane domain, and an intracellular domain. In some embodiments, the isolated TFP molecules comprises an antibody or antibody fragment comprising a human or humanized anti-TAA binding domain, a TCR extracellular domain, a transmembrane domain, and an intracellular domain. In some embodiments, the anti-TAA binding domain is a human or humanized binding domain. In some embodiments, the anti-TAA binding domain is not humanized. In some embodiments, the anti-TAA binding domain comprises a camelid antibody or an antibody fragment thereof.

In some embodiments, the antibody domain comprises an antibody fragment. In some embodiments, the antibody domain comprises a scFv, single-domain antibody (sdAb), or a V_{H} domain.

In some embodiments, the human or humanized antibody domain comprises an antibody fragment. In some embodiments, the human or humanized antibody domain comprises a scFv, single-domain antibody (sdAb), or a V_{H} domain.

In some embodiments, the anti-TAA binding domain is a scFv, a single-domain antibody (sdAb), a V_{HH} or a V_{H} domain. In other embodiments, the anti-TAA binding domain comprises a light chain and a heavy chain of an amino acid sequence provided herein, or a functional fragment thereof, or an amino acid sequence having at least one, two or three modifications but not more than 30, 20 or 10 modifications of an amino acid sequence of a light chain variable region provided herein, or a sequence with 95-99% identity with an amino acid sequence provided herein.

In some embodiments, the isolated TFP molecules comprise a TCR extracellular domain that comprises an extracellular domain or portion thereof of a protein selected from the group consisting of the alpha or beta chain of the T cell receptor, CD3 delta, CD3 epsilon, or CD3 gamma, or an amino acid sequence having at least one, two or three modifications but not more than 20, 10 or 5 modifications thereto.

In some embodiments, the anti-TAA binding domain is connected to the TCR extracellular domain by a linker sequence. In some instances, the linker region comprises (G₄S)ₙ, wherein n=1 to 4. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some embodiments, the isolated TFP molecules further comprise a sequence encoding a costimulatory domain. In other embodiments, the isolated TFP molecules further comprise a sequence encoding an intracellular signaling domain. In yet other embodiments, the isolated TFP molecules further comprise a leader sequence.

Also provided herein are vectors that comprise a nucleic acid molecule encoding any of the previously described TFP molecules. In some embodiments, the vector is selected from the group consisting of a DNA, a RNA, a plasmid, a lentivirus vector, adenoviral vector, or a retrovirus vector. In some embodiments, the vector further comprises a promoter. In some embodiments, the vector is an in vitro transcribed vector. In some embodiments, a nucleic acid sequence in the vector further comprises a poly(A) tail. In some embodiments, a nucleic acid sequence in the vector further comprises a 3′UTR.

Also provided herein are cells that comprise any of the described vectors. In some embodiments, the cell is a human T cell. In some embodiments, the cell is a CD8+ or CD4+ T cell. In other embodiments, the cells further comprise a nucleic acid encoding an inhibitory molecule that comprises a first polypeptide that comprises at least a portion of an inhibitory molecule, associated with a second polypeptide that comprises a positive signal from an intracellular signaling domain. In some instances, the inhibitory molecule comprise first polypeptide that comprises at least a portion of PD1 and a second polypeptide comprising a costimulatory domain and primary signaling domain.

In another aspect, provided herein are isolated TFP molecules that comprise an anti-TAA binding domain, a TCR extracellular domain, a transmembrane domain, and an intracellular signaling domain, wherein the TFP molecule is capable of functionally interacting with an endogenous TCR complex and/or at least one endogenous TCR polypeptide. In some embodiments, the anti-TAA binding domain is a human or humanized anti-TAA binding domain.

In another aspect, provided herein are isolated TFP molecules that comprise an anti-TAA binding domain, a TCR extracellular domain, a transmembrane domain, and an intracellular signaling domain, wherein the TFP molecule is capable of functionally integrating into an endogenous TCR complex.

In another aspect, provided herein are human CD8+ or CD4+ T cells that comprise at least two TFP molecules, the TFP molecules comprising a human or humanized anti-TAA binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein are protein complexes that comprise i) a TFP molecule comprising a human or humanized anti-TAA binding domain, a TCR extracellular domain, a transmembrane domain, and an intracellular domain; and ii) at least one endogenous TCR complex.

In some embodiments, the TCR comprises an extracellular domain or portion thereof of a protein selected from the group consisting of the alpha or beta chain of the T cell receptor, CD3 delta, CD3 epsilon, or CD3 gamma. In some embodiments, the anti-TAA binding domain is connected to the TCR extracellular domain by a linker sequence. In some instances, the linker region comprises (G₄S)ₙ, wherein n=1 to 4. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

Also provided herein are human CD8+ or CD4+ T cells that comprise at least two different TFP proteins per any of the described protein complexes.

In another aspect, provided herein is a population of human CD8+ or CD4+ T cells, wherein the T cells of the population individually or collectively comprise at least two TFP molecules, the TFP molecules comprising a human or humanized anti-TAA binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein is a population of human CD8+ or CD4+ T cells, wherein the T cells of the population individually or collectively comprise at least two TFP molecules encoded by an isolated nucleic acid molecule provided herein.

In another aspect, provided herein are methods of making a cell comprising transducing a T cell with any of the described vectors.

In another aspect, provided herein are methods of generating a population of RNA-engineered cells that comprise introducing an in vitro transcribed RNA or synthetic RNA into a cell, where the RNA comprises a nucleic acid encoding any of the described TFP molecules.

In another aspect, provided herein are methods of providing an anti-tumor immunity in a mammal that comprise administering to the mammal an effective amount of a cell expressing any of the described TFP molecules. In some embodiments, the cell is an autologous T cell. In some embodiments, the cell is an allogeneic T cell. In some embodiments, the mammal is a human.

In another aspect, provided herein are methods of treating a mammal having a disease associated with expression of a tumor associated antigen (TAA) (e.g., MUC16, IL13Rα2, or MSLN) that comprise administering to the mammal an effective amount of the cell of comprising any of the described TFP molecules. In some embodiments, the disease associated with the TAA (e.g., MUC16, IL13Rα2, MSLN) expression is selected from a proliferative disease such as a cancer or malignancy or a precancerous condition such as a pancreatic cancer, an ovarian cancer, a stomach cancer, mesothelioma, a lung cancer, or an endometrial cancer, or is a non-cancer related indication associated with expression of the TAA (e.g., MUC16, IL13Rα2, MSLN).

In some embodiments, the cells expressing any of the described TFP molecules are administered in combination with an agent that ameliorates one or more side effects associated with administration of a cell expressing a TFP molecule. In some embodiments, the cells expressing any of the described TFP molecules are administered in combination with an agent that treats the disease associated with the TAA (e.g., MUC16, IL13Rα2, MSLN).

Also provided herein are any of the described isolated nucleic acid molecules, any of the described isolated polypeptide molecules, any of the described isolated TFPs, any of the described protein complexes, any of the described vectors or any of the described cells for use as a medicament.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. A “TFP T cell” is a T cell that has been transduced according to the methods disclosed herein and that expresses a TFP, e.g., incorporated into the natural TCR. In some embodiments, the T cell is a CD4+ T cell, a CD8+ T cell, or a CD4+/CD8+ T cell. In some embodiments, the TFP T cell is an NK cell. In some embodiments,

As used herein, the term “MUC16” also known as mucin 16 or CA125 (cancer antigen 125, carcinoma antigen 125, or carbohydrate antigen 125), refers to a protein that in humans is encoded by the MUC16 gene. MUC16 is a member of the mucin family glycoproteins and has found application as a tumor marker or biomarker that may be elevated in the blood of some patients with specific types of cancers or other conditions that are benign. MUC16 is used as a biomarker for ovarian cancer detection and has been found to be elevated in other cancers, including endometrial cancer, fallopian tube cancer, lung cancer, breast cancer and gastrointestinal cancer. MUC16 has also been shown to suppress the activity of natural killer cells in the immune response to cancer cells (see, e.g., Patankar et al., Gynecologic Oncology 99(3); 704-13).

As used herein, the term “IL13Rα2”, also known as cluster of differentiation 213A2 (CD213A2), refers to a membrane-bound protein that in humans is encoded by the IL13Rα2 gene. IL13Rα2 is a subunit of the interleukin 13 receptor complex and is a receptor of the IL13 protein. IL13Rα2 has been found to be over-expressed in a variety of cancers, including pancreatic, ovarian, melanomas, and malignant gliomas.

As used herein, the term “MSLN” or “mesothelin” refers to a 40 kDa cell-surface glycosylphosphatidylinositol (GPI)-linked glycoprotein. The human mesothelin protein is synthesized as a 69 kD precursor which is then proteolytically processed. The 30 kD amino terminus of mesothelin is secreted and is referred to as megakaryocyte potentiating factor (Yamaguchi et al., J. Biol. Chem. 269:805 808, 1994). The 40 kD carboxyl terminus remains bound to the membrane as mature mesothelin (Chang et al., Natl. Acad. Sci. USA 93:136 140, 1996; Scholler et al., Cancer Lett 247(2007), 130-136). Exemplary nucleic acid and amino acid mesothelin sequences can also be determined from the MSLN gene transcript found at (NCBI accession number NM_005823 or NCBI accession number NM_013404. Accordingly, where the conjugate constructs disclosed herein are characterized by cross-competing with a reference antibody to mesothelin, or an epitope thereof, the mesothelin is that reported in Scholler et al., Cancer Lett 247(2007), 130-136.

The human and murine amino acid and nucleic acid sequences can be found in a public database, such as GenBank, UniProt and Swiss-Prot. For example, the amino acid sequence of human MUC16 can be found as UniProt/Swiss-Prot Accession No. Q8WXI7. The nucleotide sequence encoding human MUC16 can be found at Accession No. NM_024690. The nucleotide sequence encoding human MUC16 transcript variant X1 can be found at Accession No. XM_017027486. The nucleotide sequence encoding human MUC16 transcript variant X2 can be found at Accession No. XM_017027487. The nucleotide sequence encoding human MUC16 transcript variant X3 can be found at Accession No. XM_017027488. The nucleotide sequence encoding human MUC16 transcript variant X4 can be found at Accession No. XM_017027489. The nucleotide sequence encoding human MUC16 transcript variant X5 can be found at Accession No. XM_017027490. The nucleotide sequence encoding human MUC16 transcript variant X6 can be found at Accession No. XM_017027491. The nucleotide sequence encoding human MUC16 transcript variant X7 can be found at Accession No. XM_017027492. The nucleotide sequence encoding human MUC16 transcript variant X8 can be found at Accession No. XM_017027493. The nucleotide sequence encoding human MUC16 transcript variant X9 can be found at Accession No. XM_017027494. The nucleotide sequence encoding human MUC16 transcript variant X10 can be found at Accession No. XM_017027495. The nucleotide sequence encoding human MUC16 transcript variant X11 can be found at Accession No. XM_017027499. The nucleotide sequence encoding human MUC16 transcript variant X12 can be found at Accession No. XM_017027500. The nucleotide sequence encoding human MUC16 transcript variant X13 can be found at Accession No. XM_017027501. In one example, the antigen-binding portion of TFPs recognizes and binds an epitope within the extracellular domain of the MUC16 protein as expressed on a glioma cell, glioma initiating cell, normal or malignant mesothelioma cell, ovarian cancer cell, pancreatic adenocarcinoma cell, or squamous cell carcinoma cell.

The amino acid sequence of human IL13Rα2 can be found as UniProt/Swiss-Prot Accession No. Q14627. The nucleotide sequence encoding human IL13Rα2 can be found at Accession No. NM_000640. The nucleotide sequence encoding human IL13Rα2 precursor can be found at Accession No. NP_000631. In one example, the antigen-binding portion of TFPs recognizes and binds an epitope within the extracellular domain of the IL13Rα2 protein as expressed on a glioma cell, glioma initiating cell, normal or malignant mesothelioma cell, ovarian cancer cell, pancreatic adenocarcinoma cell, or squamous cell carcinoma cell.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” (or, in the case of single domain antibodies, e.g., nanobodies, “V_{HH}”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the invention comprising an antibody or antibody fragment thereof may exist in a variety of forms where the antigen binding domain is expressed as part of a contiguous polypeptide chain including, for example, a single domain antibody fragment (sdAb) or heavy chain antibodies HCAb, a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the present disclosure comprises an antibody fragment. In a further aspect, the TFP comprises an antibody fragment that comprises a scFv or a sdAb.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lung cancer, and the like.

The phrase “disease associated with expression of” MUC16, IL13Rα2, or MSLN includes, but is not limited to, a disease associated with expression of MUC16, IL13Rα2, or MSLN or condition associated with cells which express MUC16, IL13Rα2, or MSLN including, e.g., proliferative diseases such as a cancer or malignancy or a precancerous condition. In one aspect, the cancer is a glioblastoma. In one aspect, the cancer is a mesothelioma. In one aspect, the cancer is a pancreatic cancer. In one aspect, the cancer is an ovarian cancer. In one aspect, the cancer is a brain cancer. In one aspect, the cancer is a stomach cancer. In one aspect, the cancer is a lung cancer. In one aspect, the cancer is an endometrial cancer. Non-cancer related indications associated with expression of MUC16, IL13Rα2, or MSLN include, but are not limited to, e.g., autoimmune disease, (e.g., lupus, rheumatoid arthritis, colitis), inflammatory disorders (allergy and asthma), and transplantation.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the invention can be replaced with other amino acid residues from the same side chain family and the altered TFP can be tested using the functional assays described herein.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

An “intracellular signaling domain,” as the term is used herein, refers to an intracellular portion of a molecule. The intracellular signaling domain generates a signal that promotes an immune effector function of the TFP containing cell, e.g., a TFP-expressing T cell. Examples of immune effector function, e.g., in a TFP-expressing T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD66d, DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that may be required for an efficient immune response. Costimulatory molecules include, but are not limited to an MHC class 1 molecule, BTLA and a Toll ligand receptor, as well as DAP10, DAP12, CD30, LIGHT, OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, that can initiate the specific transcription of a polynucleotide sequence.

The term “promoter/regulatory sequence” refers to a nucleic acid sequence which can be used for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the invention are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that may be required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, glioblastoma, mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, and stomach cancer.

In some instances, the disease is a cancer selected from the group consisting of mesothelioma, glioblastoma, papillary serous ovarian adenocarcinoma, clear cell ovarian carcinoma, mixed Mullerian ovarian carcinoma, endometroid mucinous ovarian carcinoma, malignant pleural disease, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with MUC16, IL13Rα2, or MSLN expression, and any combination thereof.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., MUC16, IL13Rα2, or MSLN) present in a sample, but which does not necessarily and substantially recognize or bind other molecules in the sample.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### DESCRIPTION

Provided herein are compositions of matter and methods of use for the treatment of a disease such as cancer, using T cell receptor (TCR) fusion proteins. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one costimulatory molecule such as 4-1BB (i.e., CD137), CD27 and/or CD28.

### MUC16

MUC16 is a tumor associated antigen polypeptide, expressed by the human ocular surface epithelia in the mucosa of the bronchus, fallopian tube, and uterus. One proposed function of MUC16 can be to provide a protective, lubricating barrier against particles and infectious agents at mucosal surfaces. Highly polymorphic, MUC16 is composed of three domains, a Ser-/Thr-rich N-terminal domain, a repeat domain of between eleven and more than 60 partially conserved tandem repeats of on average 156 amino acids each, and a C-terminal non-repeating domain containing a transmembrane sequence and a short cytoplasmic tail. MUC16 may be heavily 0-glycosylated and N-glycosylated. mRNA encoding the MUC16 polypeptide expressed from the MUC16 gene can be significantly, reproducibly and detectably overexpressed in certain types of human cancerous ovarian, breast and pancreatic tumors as compared to the corresponding normal human ovarian, breast and pancreatic tissues, respectively. A variety of independent and different types of cancerous human ovarian tissue samples quantitatively analyzed for MUC16 expression show the level of expression of MUC16 in the cancerous samples can be variable, with a significant number of the cancerous samples showing an at least 6-fold (to as high as an about 580-fold) increase in MUC16 expression when compared to the mean level of MUC16 expression for the group of normal ovarian tissue samples analyzed. In particular, detectable and reproducible MUC16 overexpression can be observed for ovarian cancer types; endometrioid adenocarcinoma, serous cystadenocarcinoma, including papillary and clear cell adenocarcinoma, as compared to normal ovarian tissue. Due to its overexpression in certain human tumors, the MUC16 polypeptide and the nucleic acid encoding that polypeptide are targets for quantitative and qualitative comparisons among various mammalian tissue samples. The expression profiles of MUC16 polypeptide, and the nucleic acid encoding that polypeptide, can be exploited for the diagnosis and therapeutic treatment of certain types of cancerous tumors in mammals.

CA125 (Carcinoma antigen 125 (0772P, CA-0772P, CA-125) is an extracellular shed protein encoded by the MUC16 gene, and a serum marker used routinely to monitor patients with ovarian cancer. CA125 is a mullerian duct differentiation antigen that is overexpressed in epithelial ovarian cancer cells and secreted into the blood, although its expression may not be entirely confined to ovarian cancer. Serum CA125 levels can be elevated in about 80% of patients with epithelial ovarian cancer (EOC) but in less than 1% of healthy women. CA125 is a giant mucin-like glycoprotein present on the cell surface of tumor cells associated with beta-galactoside-binding, cell-surface lectins, which can be components of the extracellular matrix implicated in the regulation of cell adhesion, apoptosis, cell proliferation and tumor progression. High serum concentration of CA125 can be typical of serous ovarian adenocarcinoma, whereas it is not elevated in mucinous ovarian cancer. CA125 may not be recommended for ovarian cancer screening because normal level may not exclude tumor. However, CA125 detection can be a standard tool in monitoring clinical course and disease status in patients who have histologically confirmed malignancies. Numerous studies have confirmed the usefulness of CA125 levels in monitoring the progress of patients with EOC, and as a cancer serum marker. A rise in CA125 levels typically can precede clinical detection by about 3 months. During chemotherapy, changes in serum CA125 levels can correlate with the course of the disease. CA125 can be used as a surrogate marker for clinical response in trials of new drugs. On the other hand, CA125 may not be useful in the initial diagnosis of EOC because of its elevation in a number of benign conditions. The CA125-specific antibody MAb-B43.13 (oregovomab, OvaRex MAb-B43.13) was in clinical trials for patients with ovarian carcinoma as an immunotherapeutic agent.

MUC16 (CA-125) can play a role in advancing tumorigenesis and tumor proliferation by several different mechanisms. One way that MUC16 helps the growth of tumors can be by suppressing the response of natural killer cells, thereby protecting cancer cells from the immune response. Further evidence that MUC16 can protect tumor cells from the immune system may be the discovery that the heavily glycosylated tandem repeat domain of MUC16 can bind to galectin-1 (an immunosuppressive protein). MUC16 can participate in cell-to-cell interactions that enable the metastasis of tumor cells. This can be supported by evidence showing that MUC16 can bind selectively to mesothelin, a glycoprotein normally expressed by the mesothelial cells of the peritoneum (the lining of the abdominal cavity). MUC16 and mesothelin interactions may provide the first step in tumor cell invasion of the peritoneum. Mesothelin has also been found to be expressed in several types of cancers including mesothelioma, ovarian cancer and squamous cell carcinoma. Since mesothelin is also expressed by tumor cells, MUC16 and mesothelial interactions may aid in the gathering of other tumor cells to the location of a metastasis, thus increasing the size of the metastasis. Evidence suggests that expression of the cytoplasmic tail of MUC16 can enable tumor cells to grow, promote cell motility and may facilitate invasion. This appears to be due to the ability of the C-terminal domain of MUC16 to facilitate signaling that leads to a decrease in the expression of E-cadherin and increase the expression of N-cadherin and vimentin, which can be expression patterns consistent with epithelial-mesenchymal transition. MUC16 may also play a role in reducing the sensitivity of cancer cells to drug therapy. For example, overexpression of MUC16 can protect cells from the effects of genotoxic drugs, such as cisplatin.

### IL13Rα2

Interleukin-13 is an immune microenvironment regulator during an immune response under normal physiological conditions and also in cancer. IL-13 binds to two different receptors IL13Rα1 and IL13Rα2. In most cells, IL-13 binds to the receptor IL13Rα1 monomer with a low affinity and binds IL4Ra to form a heterodimer complex leading to downstream pathway activation of signal transducer and activator of transcription (STAT)6. IL-13 binds to the IL13Rα2 receptor in some normal cells such as testis cells but it also binds the IL13Rα2 receptor in cancer cells with high affinity.

The RNA transcript for the IL13Rα2 gene that is located in Xq13.1-q28 encodes for a 380-amino-acid protein that includes a 26-amino-acid signaling sequence and a short 17-amino-acid intracellular domain. In a glioblastoma cell, IL13Rα2 expresses up to 30,000 binding sites for IL-13 protein.

One proposed function of IL13Rα2 is as a decoy receptor which leads to sequestration of IL-13 away from IL13Rα1. As IL13Rα2 binds available IL-13 with higher affinity and provides more binding sites as compared to IL13Rα1, sequestration of IL-13 is promoted in cells. In normal cells, IL-13 binding to IL13Rα1 activates STAT6, which translocates to the nucleus, where it exerts transcriptional control over genes containing the N6-growth arrest specific promoter, such as 15-lipooxygenase-1. This may lead to apoptosis through increased caspase-3 activity. IL-13 sequestration can thus be an apoptosis escape mechanism of tumor cells. Another proposed function of IL13Rα2 is the blocking of IL13Rα1 by IL13Rα2 by physical blocking of the docking of STST6 to the receptor. The lack of STAT6 docking impedes downstream activation of apoptosis. IL13Rα2 also induces upregulation of STAT3 and B-cell lymphoma 2 in glioma cells.

IL13Rα2 is expressed on glioma initiating cells and is expressed in about 58% of adult and about 83% of pediatric brain tumors. In ovarian and pancreatic cancers, it promotes invasion and metastasis via the pathway of extracellular signal-regulated kinase/activator protein 1. Expression of IL13Rα2 in immune cells, such as myeloid derived suppressor cells, also promotes tumor immune escape and progression via upregulation of transforming growth factor 3. Increased expression of IL13Rα2 may promote tumor progression in glioma and other tumor models. Expression of IL13Rα2 increases with glioma malignancy grade and thus may provide a prognostic indicator for patient survival. The expression profiles of IL13Rα2 polypeptide and the nucleic acid encoding that polypeptide, can be exploited for the diagnosis and therapeutic treatment of certain types of cancerous tumors in mammals.

### T Cell Receptor (TCR) Fusion Proteins (TFP)

The present disclosure encompasses recombinant DNA constructs encoding TFPs, wherein the TFP comprises an antibody fragment that binds specifically to MUC16, IL13Rα2, or MSLN, e.g., human MUC16, IL13Rα2, or MSLN, wherein the sequence of the antibody fragment is contiguous with and in the same reading frame as a nucleic acid sequence encoding a TCR subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the present disclosure comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the invention include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

In one aspect, the portion of the TFP comprising the antigen binding domain comprises an antigen binding domain that targets MUC16, IL13Rα2, or MSLN. In one aspect, the antigen binding domain targets human MUC16, IL13Rα2, or MSLN.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a humanized or human antibody or an antibody fragment, or a camelid antibody or antibody fragment, or a murine antibody or antibody fragment. In one embodiment, the humanized or human anti-TAA binding domain comprises one or more (e.g., all three) light chain complementary determining region 1 (LC CDR1), light chain complementary determining region 2 (LC CDR2), and light chain complementary determining region 3 (LC CDR3) of a humanized or human anti-TAA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-TAA binding domain described herein, e.g., a humanized or human anti-TAA binding domain comprising one or more, e.g., all three, LC CDRs and one or more, e.g., all three, HC CDRs. In one embodiment, the humanized or human anti-TAA binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized or human anti-TAA binding domain described herein, e.g., the humanized or human anti-TAA (binding domain has two variable heavy chain regions, each comprising a HC CDR1, a HC CDR2 and a HC CDR3 described herein. In one embodiment, the humanized or human anti-TAA binding domain comprises a humanized or human light chain variable region described herein and/or a humanized or human heavy chain variable region described herein. In one embodiment, the humanized or human anti-TAA binding domain comprises a humanized heavy chain variable region described herein, e.g., at least two humanized or human heavy chain variable regions described herein. In one embodiment, the anti-TAA binding domain is a scFv comprising a light chain and a heavy chain of an amino acid sequence provided herein. In an embodiment, the anti-TAA binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-TAA binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-TAA binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H4}-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the present disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human tumor associated antigens such as MUC16, IL13Rα2, or MSLN. In some embodiments, a humanized antibody or antibody fragment may have improved affinity and/or specificity of binding to human MUC16, IL13Rα2, or MSLN.

In one aspect, the anti-TAA binding domain (i.e., the MUC16, IL13Rα2, or MSLN binding domain) is characterized by particular functional features or properties of an antibody or antibody fragment. For example, in one aspect, the portion of a TFP composition of the invention that comprises an antigen binding domain specifically binds human MUC16, IL13Rα2, or MSLN. In one aspect, the present disclosure relates to an antigen binding domain comprising an antibody or antibody fragment, wherein the antibody binding domain specifically binds to a MUC16, IL13Rα2, or MSLN protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-TAA binding domain is a fragment, e.g., a single chain variable fragment (scFv). In one aspect, the anti-TAA binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof disclosed herein bind a MUC16, IL13Rα2, or MSN protein with wild-type or enhanced affinity.

Also provided herein are methods for obtaining an antibody antigen binding domain specific for a target antigen (e.g., MUC16, IL13Rα2, MLSN, or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest (e.g., MUC16, IL13Rα2, MSLN) and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding may also be required to bring the two variable regions together to form a functional epitope binding site. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Pat. No. 7,695,936, U.S. Patent Application Publication Nos. 20050100543 and 20050175606, and PCT Publication Nos. WO2006/020258 and WO2007/024715, all of which are incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

### Stability and Mutations

The stability of an anti-TAA binding domain, e.g., scFv molecules (e.g., soluble scFv) can be evaluated in reference to the biophysical properties (e.g., thermal stability) of a conventional control scFv molecule or a full length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-TAA binding domain, e.g., scFv is subsequently conferred to the entire TAA-TFP construct, leading to improved therapeutic properties of the anti-TAA TFP construct. The thermal stability of the anti-TAA binding domain, e.g., scFv can be improved by at least about 2° C. or 3° C. as compared to a conventional antibody. In one embodiment, the anti-TAA binding domain, e.g., scFv has a 1° C. improved thermal stability as compared to a conventional antibody. In another embodiment, the anti-TAA binding domain, e.g., scFv has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described below.

Mutations in scFv (arising through humanization or mutagenesis of the soluble scFv) alter the stability of the scFv and improve the overall stability of the scFv and the anti-TAA TFP construct. Stability of the humanized scFv is compared against the murine scFv using measurements such as T_{M}, temperature denaturation and temperature aggregation. In one embodiment, the anti-TAA binding domain, e.g., a scFv, comprises at least one mutation arising from the humanization process such that the mutated scFv confers improved stability to the anti-TAA TFP construct. In another embodiment, the anti-TAA binding domain, e.g., scFv comprises at least 1, 2, 3, 4, 5, 6, 7, 8, 9, 10 mutations arising from the humanization process such that the mutated scFv confers improved stability to the anti-TAA-TFP construct.

In one aspect, the antigen binding domain of the TFP comprises an amino acid sequence that is homologous to an antigen binding domain amino acid sequence described herein, and the antigen binding domain retains the desired functional properties of the anti-TAA antibody fragments described herein. In one specific aspect, the TFP composition of the invention comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the invention comprises an antibody fragment. In a further aspect, that antibody fragment comprises a scFv.

It will be understood by one of ordinary skill in the art that the antibody or antibody fragment of the present disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. The algorithm parameters for using nucleotide BLAST to determine nucleotide sequence identity may use scoring parameters with a match/mismatch score of 1,−2 and wherein the gap costs are linear. The length of the sequence that initiates an alignment or the word size in a BLAST algorithm may be set to 28 for sequence alignment. The algorithm parameters for using protein BLAST to determine a peptide sequence identity may use scoring parameters with a BLOSUM62 matrix to assign a score for aligning pairs of residues, and determining overall alignment score, wherein the gap costs may have an existence penalty of 11 and an extension penalty of 1. The matrix adjustment method to compensate for amino acid composition of sequences may be a conditional compositional score matrix adjustment. The length of the sequence that initiates an alignment or the word size in a BLAST algorithm may be set to 6 for sequence alignment.

In one aspect, the present invention contemplates modifications of the starting antibody or fragment (e.g., scFv) amino acid sequence that generate functionally equivalent molecules. For example, the V_{H} or V_{L} of an anti-TAA binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-TAA binding domain, e.g., scFv. The present invention contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this present disclosure may include at least the extracellular region(s) of e.g., the alpha, beta, gamma, delta, or zeta chain of the T cell receptor, or CD3 epsilon, CD3 gamma, or CD3 delta, or in alternative embodiments, an extracellular domain may include at least the extracellular domain of CD28, CD45, CD4, CD5, CD8, CD9, CD16, CD22, CD33, CD37, CD64, CD80, CD86, CD134, CD137, or CD154. In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. In some instances, the TCR subunit comprises a transmembrane domain comprising a transmembrane domain of a protein selected from the group consisting of a TCR alpha chain, a TCR beta chain, a TCR gamma chain, a TCR delta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications. In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. A glycine-serine doublet provides a particularly suitable linker. For example, in one aspect, the linker comprises the amino acid sequence of GGGGSGGGGS (SEQ ID NO:99). In some embodiments, the linker is encoded by a nucleotide sequence of GGTGGCGGAGGTTCTGGAGGTGGAGGTTCC (SEQ ID NO:100). Other exemplary linkers are set forth in Table 4.

### Cytoplasmic Domain

The cytoplasmic domain of the TFP can include an intracellular signaling domain, if the TFP contains CD3 gamma, delta or epsilon polypeptides; TCR alpha and TCR beta subunits are generally lacking in a signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone may be insufficient for full activation of naive T cells and that a secondary and/or costimulatory signal may be required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain can regulate primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the invention include those of CD3 zeta, FcR gamma, FcR beta, CD3 gamma, CD3 delta, CD3 epsilon, CD5, CD22, CD79a, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that may be required for an efficient response of lymphocytes to an antigen. Examples of such molecules include CD27, CD28, 4-1BB (CD137), OX40, DAP10, DAP12, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target (e.g., MUC16, IL13Rα2, MSLN) or a different target (e.g., CD123). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not associate with the antigen binding domain of the second TFP, e.g., the antigen binding domain of the second TFP is a V_{HH}. In one embodiment, the antigen binding domain is SD1 (SEQ ID NO:15), SD2 (SEQ ID NO:20), SD3 (SEQ ID NO:25), SD4 (SEQ ID NO:30), SD5 (SEQ ID NO:35), or SD6 (SEQ ID NO:40). In one embodiment, the antigen binding domain is LSD1 (SEQ ID NO:51), H1-LSD1 (SEQ ID NO:56), H2-LSD1 (SEQ ID NO:61), LSD2 (SEQ ID NO:66), H1-LSD1 (SEQ ID NO:71), or H2-LSD2 (SEQ ID NO:76). In one embodiment, the antigen binding domain is anti-MSLN VHH1 (SEQ ID NO:96) or anti-MSLN VHH2 (SEQ ID NO:97).

In another aspect, the TFP-expressing cell described herein can further express another agent, e.g., an agent which enhances the activity of a TFP-expressing cell. For example, in one embodiment, the agent can be an agent which inhibits an inhibitory molecule. Inhibitory molecules, e.g., PD1, can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD1 can be expressed on activated B cells, T cells and myeloid cells (Agata et al. 1996 Int. Immunol 8:765-75). Two ligands for PD1, Programmed Death-Ligand 1 (PD-L1) and Programmed Death-Ligand 2 (PD-L2) have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 can be abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-TAA TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the PD-L1 or PD-L2.

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having an anti-TAA binding domain described herein, and a second cell expressing a TFP having a different anti-TAA binding domain, e.g., an anti-TAA binding domain described herein that differs from the anti-TAA binding domain in the TFP expressed by the first cell. As another example, the population of TFP-expressing cells can include a first cell expressing a TFP that includes an anti-TAA binding domain, e.g., as described herein, and a second cell expressing a TFP that includes an antigen binding domain to a target other than the anti-TAA TFP of the first cell (e.g., with specificity for MUC16, IL13Rα2, or MSLN) (e.g., another tumor-associated antigen).

In another aspect, the present disclosure provides a population of cells wherein at least one cell in the population expresses a TFP having an anti-TAA domain described herein, and a second cell expressing another agent, e.g., an agent which enhances the activity of a TFP-expressing cell. For example, in one embodiment, the agent can be an agent which inhibits an inhibitory molecule. Inhibitory molecules, e.g., can, in some embodiments, decrease the ability of a TFP-expressing cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present invention also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect, the anti-TAA TFP is encoded by a messenger RNA (mRNA). In one aspect the mRNA encoding the anti-TAA TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present invention. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3,000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths that can be used to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 Ts), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Nucleic Acid Constructs Encoding a TFP

The present disclosure also provides nucleic acid molecules encoding one or more TFP constructs described herein. In one aspect, the nucleic acid molecule is provided as a messenger RNA transcript. In one aspect, the nucleic acid molecule is provided as a DNA construct.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Disclosed herein, in some embodiments, are vectors comprising the recombinant nucleic acid disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector. In some embodiments, the vector is a circular RNA vector (e.g., as disclosed in co-pending Provisional Patent Application No. 62/836,977).

The present disclosure also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. Disclosed herein, in some embodiments, are vectors comprising the recombinant nucleic acid disclosed herein.

In another embodiment, the vector comprising the nucleic acid encoding the desired TFP of the present disclosure is an adenoviral vector (A5/35). In another embodiment, the expression of nucleic acids encoding TFPs can be accomplished using of transposons such as sleeping beauty, crisper, CAS9, and zinc finger nucleases (See, June et al. 2009 Nature Reviews Immunol. 9.10: 704-716, incorporated herein by reference).

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the present disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes. Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Gene Editing Technologies

In some embodiments, the modified T cells disclosed herein are engineered using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. In this way, a chimeric construct may be engineered to combine desirable characteristics of each subunit, such as conformation or signaling capabilities. See also Sander & Joung, Nat. Biotech. (2014) v32, 347-55; and June et al., 2009 Nature Reviews Immunol. 9.10: 704-716, each incorporated herein by reference. In some embodiments, one or more of the extracellular domain, the transmembrane domain, or the cytoplasmic domain of a TFP subunit are engineered to have aspects of more than one natural TCR subunit domain (i.e., are chimeric).

Recent developments of technologies to permanently alter the human genome and to introduce site-specific genome modifications in disease relevant genes lay the foundation for therapeutic applications. These technologies are now commonly known as “genome editing.

In some embodiments, gene editing techniques are employed to disrupt an endogenous TCR gene. In some embodiments, mentioned endogenous TCR gene encodes a TCR alpha chain, a TCR beta chain, or a TCR alpha chain and a TCR beta chain. In some embodiments, gene editing techniques pave the way for multiplex genomic editing, which allows simultaneous disruption of multiple genomic loci in endogenous TCR gene. In some embodiments, multiplex genomic editing techniques are applied to generate gene-disrupted T cells that are deficient in the expression of endogenous TCR, and/or human leukocyte antigens (HLAs), and/or programmed cell death protein 1 (PD1), and/or other genes.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or -when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a predetermined DNA sequence −18 basepairs in length. By fusing this engineered protein domain to the Fokl nuclease, it is possible to target DNA breaks with genome-level specificity. ZFNs have been used extensively to target gene addition, removal, and substitution in a wide range of eukaryotic organisms (reviewed in Durai et al. (2005), Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fokl nuclease domain (reviewed in Mak et al. (2013), Curr Opin Struct Biol. 23:93-9). In this case, however, the DNA binding domain comprises a tandem array of TAL-effector domains, each of which specifically recognizes a single DNA basepair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-TevI homing endonuclease. Unlike Fokl, I-TevI does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or a RNA duplex comprising a 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing). There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally-occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy. 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, must be efficiently delivered to the cells of interest. Delivery methods such as physical, chemical, and viral methods are also know in the art (Mali (2013). Indian J. Hum. Genet. 19: 3-8.). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

The present invention further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian T cells. In one aspect, the mammalian T cell is a human T cell.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present invention, any number of T cell lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, and 7,572,631.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-TAA TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-TAA TFP are described in further detail below.

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by Western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0, and transduced with TFP on day 1 using a bicistronic lentiviral vector expressing TFP along with eGFP using a 2A ribosomal skipping sequence. Cultures are re-stimulated with either TAA+ cells (e.g., K562 cells) wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human TAA-specific TFP+ T cells to treat a cancer in immunodeficient mice (see, e.g., Milone et al., Molecular Therapy 17(8): 1453-1464 (2009)). Very briefly, after establishment of cancer, mice are randomized as to treatment groups. Different numbers of engineered T cells are coinjected at a 1:1 ratio into NOD/SCID/γ−/− mice bearing cancer. The number of copies of each vector in spleen DNA from mice is evaluated at various times following T cell injection. Animals are assessed for cancer at weekly intervals. Peripheral blood TAA+ cancer cell counts are measured in mice that are injected with alpha-TAA-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/−mice can also be analyzed. Mice are injected with cancer cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for cancer at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing cancer in mice injected on day 21 with TFP T cells, an equivalent number of mock-transduced T cells, or no T cells. Mice from each group are randomly bled for determination of peripheral blood TAA+ cancer cell counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with cells expressing TAA or CD32 and CD137 (KT32-BBL) for a final T cell:cell expressing TAA ratio of 2:1. Cells expressing TAA are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant TAA protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur flow cytometer, and data is analyzed according to the manufacturer's instructions.

Cytotoxicity can be assessed by a standard ⁵¹Cr-release assay (see, e.g., Milone et al., Molecular Therapy 17(8): 1453-1464 (2009)). Briefly, target cells are loaded with ⁵¹Cr (as NaCrO₄, New England Nuclear) at 37° C. for 2 hours with frequent agitation, washed twice in complete RPMI medium and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER-SR)/(TR-SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). Briefly, NOD/SCID/γc−/− (NSG) mice are injected IV with cancer cells followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in a cancer xenograft model can be measured as follows: NSG mice are injected with cancer cells transduced to stably express firefly luciferase, followed by a single tail-vein injection of T cells electroporated with TAA TFP 7 days later. Animals are imaged at various time points post injection. For example, photon-density heat maps of firefly luciferase positive cancer in representative mice at day 5 (2 days before treatment) and day 8 (24 hours post TFP+ PBLs) can be generated.

Other assays, including those described in the Example section herein as well as those that are known in the art can also be used to evaluate the anti-TAA TFP constructs of the present disclosure.

### Therapeutic Applications

MUC16, IL13Rα2, and MSLN Associated Diseases and/or Disorders

In one aspect, the present disclosure provides methods for treating a disease associated with MUC16, IL13Rα2, or MSLN expression. In one aspect, the present disclosure provides methods for treating a disease wherein part of the tumor is negative for MUC16, IL13Rα2, or MSLN and part of the tumor is positive for MUC16, IL13Rα2, or MSLN. For example, the TFP of the present disclosure is useful for treating subjects that have undergone treatment for a disease associated with elevated expression of MUC16, IL13Rα2, or MSLN, wherein the subject that has undergone treatment for elevated levels of MUC16, IL13Rα2, or MSLN exhibits a disease associated with elevated levels of MUC16, IL13Rα2, or MSLN.

In one aspect, the present disclosure pertains to a vector comprising anti-TAA TFP operably linked to promoter for expression in mammalian T cells. In one aspect, the present disclosure provides a recombinant T cell expressing the MUC16, IL13Rα2, or MSLN TFP for use in treating MUC16, IL13Rα2, or MSLN-expressing tumors, respectively wherein the recombinant T cell expressing the MUC16, IL13Rα2, or MSLN TFP is termed a MUC16, IL13Rα2, or MSLN TFP-T. In one aspect, the MUC16, IL13Rα2, or MSLN TFP-T of the present disclosure is capable of contacting a tumor cell with at least one MUC16, IL13Rα2, or MSLN TFP of the invention expressed on its surface such that the TFP-T targets the tumor cell and growth of the tumor is inhibited.

In one aspect, the present disclosure pertains to a method of inhibiting growth of a MUC16, IL13Rα2, or MSLN-expressing tumor cell, comprising contacting the tumor cell with a anti-MUC16, anti-IL13Rα2, or anti-MSLN TFP T cell of the present disclosure such that the TFP-T is activated in response to the antigen (e.g., the MUC16, IL13Rα2, or MSLN antigen present on the surface of the cancer cell) and targets the cancer cell, wherein the growth of the tumor is inhibited.

In one aspect, the present disclosure pertains to a method of treating cancer in a subject. The method comprises administering to the subject an anti-TAATFP T cell of the present disclosure such that the cancer is treated in the subject. An example of a cancer that is treatable by the anti-TAA TFP T cell of the invention is a cancer associated with expression of the corresponding TAA. In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is an endometrial cancer. In some embodiments, anti-TAA TFP therapy can be used in combination with one or more additional therapies.

The present disclosure includes a type of cellular therapy where T cells are genetically modified to express a TFP and the TFP-expressing T cell is infused to a recipient in need thereof. The infused cell is able to kill tumor cells in the recipient. Unlike antibody therapies, TFP-expressing T cells are able to replicate in vivo, resulting in long-term persistence that can lead to sustained tumor control. In various aspects, the T cells administered to the patient, or their progeny, persist in the patient for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The present disclosure also includes a type of cellular therapy where T cells are modified, e.g., by in vitro transcribed RNA, to transiently express a TFP and the TFP-expressing T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the TFP-expressing T cells may be an active or a passive immune response, or alternatively may be due to a direct vs indirect immune response. In one aspect, the TFP transduced T cells exhibit specific proinflammatory cytokine secretion and potent cytolytic activity in response to human cancer cells expressing the tumor associated antigen (TAA) (e.g., MUC16, IL13Rα2, or MSLN), resist soluble TAA inhibition, mediate bystander killing and/or mediate regression of an established human tumor. For example, antigen-less tumor cells within a heterogeneous field of TAA-expressing tumor may be susceptible to indirect destruction by TAA-redirected T cells that has previously reacted against adjacent antigen-positive cancer cells.

In one aspect, the human TFP-modified T cells of the present disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a TFP disclosed herein. The TFP-modified cell can be administered to a mammalian recipient to provide a therapeutic benefit. The mammalian recipient may be a human and the TFP-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the TFP-modified T cells of the invention are used in the treatment of diseases, disorders and conditions associated with expression of MUC16, IL13Rα2, or MSLN. In certain aspects, the cells of the invention are used in the treatment of patients at risk for developing diseases, disorders and conditions associated with expression of MUC16, IL13Rα2, or MSLN. Thus, the present invention provides methods for the treatment or prevention of diseases, disorders and conditions associated with expression of MUC16, IL13Rα2, or MSLN comprising administering to a subject in need thereof, a therapeutically effective amount of the TFP-modified T cells of the present disclosure.

In one aspect the TFP-T cells of the present disclosure may be used to treat a proliferative disease such as a cancer or malignancy or a precancerous condition. In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is breast cancer. In one aspect, the cancer is a endometrial cancer. Further a disease associated with MUC16, IL13Rα2, or MSLN expression includes, but is not limited to, e.g., atypical and/or non-classical cancers, malignancies, precancerous conditions or proliferative diseases expressing MUC16, IL13Rα2, or MSLN. Non-cancer related indications associated with expression of MUC16, IL13Rα2, or MSLN include, but are not limited to, e.g., autoimmune disease, (e.g., lupus), inflammatory disorders (allergy and asthma), inflammatory bowel disease, liver cirrhosis, cardiac failure, peritoneal infection, and abdominal surgery and transplantation.

The TFP-modified T cells of the present disclosure may be administered either alone, or as a pharmaceutical composition in combination with diluents and/or with other components such as IL-2 or other cytokines or cell populations.

The present invention also provides methods for inhibiting the proliferation or reducing a TAA-expressing cell population, the methods comprising contacting a population of cells comprising a TAA-expressing cell with an anti-TAA TFP-T cell of the present disclosure that binds to the TAA-expressing cell. In some aspects, the present disclosure provides methods for inhibiting the proliferation or reducing the population of cancer cells expressing TAA, the methods comprising contacting the TAA-expressing cancer cell population with an anti-TAA TFP-T cell of the present disclosure that binds to the TAA-expressing cell. In one aspect, the present disclosure provides methods for inhibiting the proliferation or reducing the population of cancer cells expressing the tumor associated antigen, the methods comprising contacting the TAA-expressing cancer cell population with an anti-TAA TFP-T cell of the present disclosure that binds to the TAA-expressing cell. In certain aspects, the anti-TAA TFP-T cell of the present disclosure reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model a cancer associated with TAA-expressing cells relative to a negative control. In one aspect, the subject is a human.

The present disclosure also provides methods for preventing, treating and/or managing a disease associated with TAA-expressing cells (e.g., a cancer expressing TAA), the methods comprising administering to a subject in need an anti-TAA TFP-T cell of the present disclosure that binds to the TAA-expressing cell. In one aspect, the subject is a human. Non-limiting examples of disorders associated with TAA-expressing cells include autoimmune disorders (such as lupus), inflammatory disorders (such as allergies and asthma) and cancers (such as pancreatic cancer, ovarian cancer, stomach cancer, lung cancer, or endometrial cancer. or atypical cancers expressing TAA).

The present disclosure also provides methods for preventing, treating and/or managing a disease associated with TAA-expressing cells, the methods comprising administering to a subject in need an anti-TAA TFP-T cell of the present disclosure that binds to the TAA-expressing cell. In one aspect, the subject is a human.

The present disclosure provides methods for preventing relapse of cancer associated with TAA-expressing cells, the methods comprising administering to a subject in need thereof an anti-TAA TFP-T cell of the present disclosure that binds to the TAA-expressing cell. In one aspect, the methods comprise administering to the subject in need thereof an effective amount of an anti-TAA TFP-T cell described herein that binds to the TAA-expressing cell in combination with an effective amount of another therapy.

### Combination Therapies

A TFP-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a TFP-expressing cell. Also provided are T cells that express multiple TFPs, which bind to the same or different target antigens, or same or different epitopes on the same target antigen. Also provided are populations of T cells in which a first subset of T cells express a first TFP and a second subset of T cells express a second TFP.

A TFP-expressing cell described herein and the at least one additional therapeutic agent can be administered simultaneously, in the same or in separate compositions, or sequentially. For sequential administration, the TFP-expressing cell described herein can be administered first, and the additional agent can be administered second, or the order of administration can be reversed.

In further aspects, a TFP-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, FK506, rapamycin, mycophenolic acid, steroids, FR901228, cytokines, and irradiation. A TFP-expressing cell described herein may also be used in combination with a peptide vaccine, such as that described in Izumoto et al. 2008 J Neurosurg 108:963-971. In a further aspect, a TFP-expressing cell described herein may also be used in combination with a promoter of myeloid cell differentiation (e.g., all-trans retinoic acid), an inhibitor of myeloid-derived suppressor cell (MDSC) expansion (e.g., inhibitors of c-kit receptor or a VEGF inhibitor), an inhibition of MDSC function (e.g., COX2 inhibitors or phosphodiesterase-5 inhibitors), or therapeutic elimination of MDSCs (e.g., with a chemotherapeutic regimen such as treatment with doxorubicin and cyclophosphamide). Other therapeutic agents that may prevent the expansion of MDSCs include amino-biphosphonate, biphosphanate, sildenafil and tadalafil, nitroaspirin, vitamin D3, and gemcitabine. (See, e.g., Gabrilovich and Nagaraj, Nat. Rev. Immunol, (2009) v9(3): 162-174).

In one embodiment, the subject can be administered an agent which reduces or ameliorates a side effect associated with the administration of a TFP-expressing cell. Side effects associated with the administration of a TFP-expressing cell include, but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a TFP-expressing cell described herein to a subject and further administering an agent to manage elevated levels of a soluble factor resulting from treatment with a TFP-expressing cell. In one embodiment, the soluble factor elevated in the subject is one or more of IFN-γ, TNFα, IL-2, IL-6 and IL8. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of a TFP-expressing cell. For example, in one embodiment, the agent can be an agent which inhibits an inhibitory molecule. Inhibitory molecules, e.g., Programmed Death 1 (PD1), can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a TFP-expressing cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a TFP-expressing cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy™; Bristol-Myers Squibb; tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the T cells may be altered (e.g., by gene transfer) in vivo via a lentivirus, e.g., a lentivirus specifically targeting a CD4+ or CD8+ T cell. (See, e.g., Zhou et al., J. Immunol. (2015) 195:2493-2501).

In some embodiments, the agent which enhances the activity of a TFP-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-TAA TFP.

### Pharmaceutical Compositions

Pharmaceutical compositions of the present disclosure may comprise a TFP-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the T cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a TFP-expressing T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded TFP T cells of the present disclosure. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the present disclosure, and one or more subsequent administrations of the TFP T cells of the present disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the present disclosure are administered to the subject (e.g., human) per week, e.g., 2, 3, or 4 administrations of the TFP T cells of the present disclosure are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the present disclosure are administered for at least two, three, four, five, six, seven, eight or more weeks.

In one aspect, TAA TFP T cells are generated using lentiviral viral vectors, such as lentivirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be effected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen-day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Cytokine Release

Cytokine release syndrome is a form of systemic inflammatory response syndrome that arises as a complication of some diseases or infections, and is also an adverse effect of some monoclonal antibody drugs, as well as adoptive T cell therapies. TFP T cells can exhibit better killing activity than CAR-T cells. TFP T cells administered to a subject can exhibit better killing activity than CAR-T cells administered to a subject. This can be one of the advantages of TFP T cells over CAR-T cells. TFP T cells can exhibit less cytokine release CAR-T cells. A subject administered TFP T cells can exhibit less cytokine release than a subject administered CAR-T cells. This can be one of the advantages of TFP T cell therapies over CAR-T cell therapies. TFP T cells can exhibit similar or better killing activity than CAR-T cells and the TFP T cells can exhibit less cytokine release than the CAR-T cells. TFP T cells administered to a subject can exhibit similar or better killing activity than CAR-T cells administered to a subject and the subject can exhibit less cytokine release than a subject administered CAR-T cells. This can be one of the advantages of TFP T cell therapies over CAR-T cell therapies.

In some cases, the cytokine release of a treatment with TFP T cells is less than the cytokine release of a treatment with CAR-T cells. In some embodiments, the cytokine release of a treatment with TFP T cells is at least 10%, at least 20%, at least 30%, at least 40%, at least 50%, at least 60%, at least 70%, at least 80%, or at least 90% less than the cytokine release of a treatment with CAR-T cells. Various cytokines can be released less in the T cell treatment with TFP T cells than CAR-T cells. In some embodiments, the cytokine is IL-2, IFN-γ, IL-4, TNF-α, IL-6, IL-13, IL-5, IL-10, sCD137, GM-CSF, MIP-1α, MIP-1β, or a combination thereof. In some cases, the treatment with TFP T cells release less perforin, granzyme A, granzyme B, or a combination thereof, than the treatment with CAR-T cells. In some embodiments, the perforin, granzyme A, or granzyme B released in a treatment with TFP T cells is at least 10%, at least 20%, at least 30%, at least 40%, at least 50%, or at least 60% less than a treatment with CAR-T cells.

In some embodiments, for a given cytokine, at least 10% less amount of the given cytokine is released following treatment compared to an amount of the given cytokine of a mammal treated with a CAR-T cell comprising the same human or humanized antibody domain. In some embodiments, the given cytokine comprises one or more cytokines selected from the group consisting of IL-2, IFN-γ, IL-4, TNF-α, IL-6, IL-13, IL-5, IL-10, sCD137, GM-CSF, MIP-1α, MIP-1β, and any combination thereof.

The TFP T cells may exhibit similar or better activity in killing tumor cells than CAR-T cells. In some embodiments, a tumor growth in the mammal is inhibited such that a size of the tumor is at most 10%, at most 20%, at most 30%, at most 40%, at most 50%, or at most 60% of a size of a tumor in a mammal treated with T cells that do not express the TFP after at least 8 days of treatment, wherein the mammal treated with T cells expressing TFP and the mammal treated with T cells that do not express the TFP have the same tumor size before the treatment. In some embodiments, the tumor growth in the mammal is completely inhibited. In some embodiments, the tumor growth in the mammal is completely inhibited for at least 20 days, at least 30 days, at least 40 days, at least 50 days, at least 60 days, at least 70 days, at least 80 days, at least 90 days, at least 100 days, or more. In some embodiments, the population of T cells transduced with TFP kill similar amount of tumor cells compared to the CAR-T cells comprising the same human or humanized antibody domain.

The TFP T cells can exhibit different gene expression profile than cells that do not express TFP. In some cases, the TFP T cells may exhibit similar gene expression profiles than CAR-T cells. In some other cases, the TFP T cells may exhibit different gene expression profiles than CAR-T cells. In some embodiments, the population of T cells transduced with TFP have a different gene expression profile than the CAR-T cells comprising the same human or humanized antibody domain. In some embodiments, an expression level of a gene is different in the T cells transduced with the TFP than an expression level of the gene in the CAR-T cells comprising the same human or humanized antibody domain. In some embodiments, the gene has a function in antigen presentation, TCR signaling, homeostasis, metabolism, chemokine signaling, cytokine signaling, toll like receptor signaling, MMP and adhesion molecule signaling, or TNFR related signaling.

### EXAMPLES

The present disclosure is further described in detail by reference to the following experimental examples. These examples are provided for purposes of illustration only, and are not intended to be limiting unless otherwise specified. Thus, the present disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present disclosure and practice the claimed methods. The following working examples specifically point out various aspects of the present disclosure, and are not to be construed as limiting in any way the remainder of the disclosure.

### Example 1: TFP Constructs

Anti-TAA TFP constructs can be engineered by cloning an anti-TAA V_{HH} domain (or SD domain) DNA fragment linked to a CD3 or TCR DNA fragment by either a DNA sequence encoding a short linker (SL): AAAGGGGSGGGGSGGGGSLE (SEQ ID NO:2) or a long linker (LL): AAAIEVMYPPPYLGGGGSGGGGSGGGGSLE (SEQ ID NO:3) into p510 vector ((System Biosciences (SBI)) at XbaI and EcoR1 sites. Other vectors may also be used, for example, pLRPO vector.

Examples of the anti-TAA TFP constructs generated include p510_anti-TAA_LL_TCRα (anti-TAA V_{HH}— long linker—human full length T cell receptor α chain), p510_TAA_LL_TCR αC (anti-TAA V_{HH}—long linker—human T cell receptor a constant domain chain), p510_anti-TAA_LL_TCRβ (anti-TAA V_{HH}— long linker—human full length T cell receptor β chain), p510_anti-TAA_LL_TCRβ C (anti-TAA V_{HH}—long linker—human T cell receptor β constant domain chain), p510_anti-TAA_LL_CD3γ (anti-TAA V_{HH}— long linker-human CD3γ chain), p510_anti-TAA_LL_CD3δ (anti-TAA V_{HH}— long linker—human CD3δ chain), p510_anti-TAA_LL_CD3ε (anti-TAA V_{HH}— long linker—human CD3ε chain), p510_anti-TAA_SL_TCRβ (anti-TAA V_{HH}—short linker—human full length T cell receptor β chain), p510_anti-TAA_SL_CD3γ (anti-TAAV_{HH}—short linker—human CD3γ chain), p510_anti-TAA_SL_CD3δ (anti-TAA V_{HH}—short linker—human CD3δ chain), p510_anti-TAA_SL_CD3ε (anti-TAA V_{HH}—short linker—human CD3ε chain).

The anti-MUC16 used herein may be a human MUC16 specific scFv, for example, 4H11.

Example of the corresponding anti-MUC16, anti-IL13Rα2, or anti-MSLN A CAR construct, p510_anti-TAA_28ζ can be generated by cloning synthesized DNA encoding the anti-TAA, partial CD28 extracellular domain, CD28 transmembrane domain, CD28 intracellular domain and CD3 zeta into p510 vector at XbaI and EcoR1 sites.

Various other vector may be used to generate fusion protein constructs.

### Example 2: Antibody Sequences

### Generation of Antibody Sequences

Generation of scFvs

Human or humanized anti-TAA IgGs can be used to generate scFv sequences for TFP constructs. DNA sequences coding for human or humanized V_{L} and V_{H} domains can be obtained, and the codons for the constructs can be, optionally, optimized for expression in cells from Homo sapiens. The order in which the V_{L} and V_{H} domains appear in the scFv is varied (i.e., V_{L}-V_{H}, or V_{H}-V_{L} orientation), and three copies of the “G4S” or “G₄S” subunit (G₄S)₃ connect the variable domains to create the scFv domain. Anti-TAA scFv plasmid constructs can have optional Flag, His or other affinity tags, and can be electroporated into HEK293 or other suitable human or mammalian cell lines and purified. Validation assays include binding analysis by FACS, kinetic analysis using Proteon, and staining of MUC16-, IL13Rα2-, or MSLN-expressing cells.

Examples of anti-MUC16, anti-IL13Rα2, or anti-MSLN binding domains, including V_{L} domain, V_{H} domain, and CDRs, that can be used with the compositions and methods described herein can be in some publications and/or commercial sources. For example, Certain anti-MUC16 antibodies, including 3A5 and 11D10, have been disclosed in WO 2007/001851, die contents of which are incorporated by reference. The 3A5 monoclonal antibody binds multiple sites of the MUC16 polypeptide with 433 pM affinity by OVCAR-3 Scatchard analysis. Examples of VL and VH domains, CDRs and the nucleotide sequences encoding them, respectively, can be those of the following monoclonal antibodies: GTX10029, GTX21107. MA5-124525, MA5-11579, 25450002, ABIN1584127, ABIN93655, 112889, 120204, LS-C356195, LS-B6756, TA801241, T A801279, V3494, V3648, 666902, 666904, HPA065600, AMAb91056.

The human IL13Rα2 polypeptide canonical sequence is UniProt Accession No. Q14627. Provided are antibody polypeptides that are capable of specifically binding to the human MUC16, IL13Rα2, or MSLN polypeptide, and fragments or domains thereof. Anti-TAA antibodies can be generated using diverse technologies (see, e.g., Nicholson et al, 1997). Where murine anti-TAA antibodies are used as a starting material, humanization of murine anti-TAA antibodies is desired for the clinical setting, where the mouse-specific residues may induce a human-anti-mouse antigen (HAMA) response in subjects who receive T cell receptor (TCR) fusion protein (TFP) treatment, i.e., treatment with T cells transduced with the TFP.TAA construct. Humanization is accomplished by grafting CDR regions from murine anti-TAA antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

### Single Domain Binders

Camelid and other single domain antibodies can also be used to generate anti-MUC16, IL13Rα2, MSLN, or other anti-tumor antigen TFP constructs. The V_{HH} domain can be used to be fused with various TCR subunits. In some embodiments, single-domain (e.g., V_{HH}) binders are used such as those set forth in Table 4 (see, e.g., non-limiting examples of SEQ ID NO:15, SEQ ID NO:20, SEQ ID NO:25, SEQ ID NO:30, SEQ ID NO:35, SEQ ID NO:40, SEQ ID NO:44, SEQ ID NO:48, SEQ ID NO:51, SEQ ID NO:56, SEQ ID NO:61, SEQ ID NO:66, SEQ ID NO:71, SEQ ID NO:76, SEQ ID NO:97, OR SEQ ID NO:98). The preparation of anti-TAA single domain antibodies is further described in Examples 3 and 5.

### Source of TCR Subunits

Subunits of the human T Cell Receptor (TCR) complex all contain an extracellular domain, a transmembrane domain, and an intracellular domain. A human TCR complex contains the CD3-epsilon polypeptide, the CD3-gamma polypeptide, the CD3-delta polypeptide, the CD3-zeta polypeptide, the TCR alpha chain polypeptide and the TCR beta chain polypeptide. The human CD3-epsilon polypeptide canonical sequence is Uniprot Accession No. P07766. The human CD3-gamma polypeptide canonical sequence is Uniprot Accession No. P09693. The human CD3-delta polypeptide canonical sequence is Uniprot Accession No. P043234. The human CD3-zeta polypeptide canonical sequence is Uniprot Accession No. P20963. The human TCR alpha chain canonical sequence is Uniprot Accession No. Q6ISU1. The human TCR beta chain C region canonical sequence is Uniprot Accession No. P01850, a human TCR beta chain V region sequence is P04435.

The human CD3-epsilon polypeptide canonical sequence is:

(SEQ ID NO: 4)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATMVDICITGGLLLLVYYWS
KNRKAKAKPVTRGAGAGGRQRGQNKERPPPVPNPDYEPIRKGQRDLYSGL
NQRRI.

The human CD3-gamma polypeptide canonical sequence is:

(SEQ ID NO: 5)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

The human CD3-delta polypeptide canonical sequence is:

(SEQ ID NO: 6)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNKS.

The human CD3-zeta polypeptide canonical sequence is:

(SEQ ID NO: 7)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR.

The human TCR alpha chain canonical sequence is:

(SEQ ID NO: 8)
MAGTWLLLLLALGCPALPTGVGGTPFPSLAPPIMLLVDGKQQMVVVCLVL
DVAPPGLDSPIWFSAGNGSALDAFTYGPSPATDGTWTNLAHLSLPSEELA
SWEPLVCHTGPGAEGHSRSTQPMHLSGEASTARTCPQEPLRGTPGGALWL
GVLRLLLFKLLLFDLLLTCSCLCDPAGPLPSPATTTRLRALGSHRLHPAT
ETGGREATSSPRPQPRDRRWGDTPPGRKPGSPVWGEGSYLSSYPTCPAQA
WCSRSALRAPSSSLGAFFAGDLPPPLQAGAA.

The human TCR alpha chain C region canonical sequence is:

(SEQ ID NO: 9)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGFNLLMTLRLWSS.

The human TCR alpha chain V region CTL-L17 canonical sequence is:

(SEQ ID NO: 10)
MAMLLGASVLILWLQPDWVNSQQKNDDQQVKQNSPSLSVQEGRISILNCD
YTNSMFDYFLWYKKYPAEGPTFLISISSIKDKNEDGRFTVFLNKSAKHLS
LHIVPSQPGDSAVYFCAAKGAGTASKLTFGTGTRLQVTL.

The human TCR beta chain C region canonical sequence is:

(SEQ ID NO: 11)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF.

The human TCR beta chain V region CTL-L17 canonical sequence is:

(SEQ ID NO: 12)
MGTSLLCWMALCLLGADHADTGVSQNPRHNITKRGQNVTFRCDPISEHNR
LYWYRQTLGQGPEFLTYFQNEAQLEKSRLLSDRFSAERPKGSFSTLEIQR
TEQGDSAMYLCASSLAGLNQPQHFGDGTRLSIL.

The human TCR beta chain V region YT35 canonical sequence is:

(SEQ ID NO: 13)
MDSWTFCCVSLCILVAKHTDAGVIQSPRHEVTEMGQEVTLRCKPISGHNS
LFWYRQTMMRGLELLIYFNNNVPIDDSGMPEDRFSAKMPNASFSTLKIQP
SEPRDSAVYFCASSFSTCSANYGYTFGSGTRLTVV.

Generation of TFPs from TCR Domains and scFvs

The MUC16, IL13Rα2, or MSLN scFvs can be recombinantly linked to CD3-epsilon or other TCR subunits using a linker sequence, such as G₄S, (G₄S)₂ (G₄S)₃ or (G₄S)₄. Various linkers and scFv configurations can be utilized. TCR alpha and TCR beta chains can be used for generation of TFPs either as full length polypeptides or only their constant domains. Any variable sequence of TCR alpha and TCR beta chains can be allowed for making TFPs.

### TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

The TFP-encoding nucleic acid construct can be cloned into a lentiviral expression vector and expression validated based on the quantity and quality of the effector T cell response of TFP.TAA-transduced T cells (“TAA.TFP” or “TAA.TFP T cells” or “TFP.TAA” or “TFP.TAA T cells”) in response to TAA+ target cells, wherein ‘TAA’ is, e.g., MUC16, IL13Ra2, or MSLN. Effector T cell responses include, but are not limited to, cellular expansion, proliferation, doubling, cytokine production and target cell lysis or cytolytic activity (i.e., degranulation).

The anti-TAA TFP lentiviral transfer vectors can be used to produce the genomic material packaged into the VSV-G pseudotyped lentiviral particles. Lentiviral transfer vector DNA is mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media is collected, filtered and concentrated by ultracentrifugation. The resulting viral preparation is stored at −80° C. The number of transducing units can be determined by titration on Sup-T1 (T cell lymphoblastic lymphoma, ATCC® CRL-1942™) cells. Redirected TFP T cells are produced by activating fresh naïve T cells with, e.g., anti-CD3 anti-CD28 beads for 24 hrs and then adding the appropriate number of transducing units to obtain the desired percentage of transduced T cells. These modified T cells will be allowed to expand until they become rested and come down in size at which point they are cryopreserved for later analysis. The cell numbers and sizes are measured using a Coulter Multisizer™ III. Before cryopreserving, the percentage of cells transduced (expressing the TFP on the cell surface) and the relative fluorescence intensity of that expression will be determined by flow cytometric analysis. From the histogram plots, the relative expression levels of the TFPs can be examined by comparing percentage transduced with their relative fluorescent intensity.

In some embodiments, multiple TFPs are introduced by T cell transduction with multiple viral vectors.

### Evaluating Cytolytic Activity, Proliferation Capabilities and Cytokine Secretion of Humanized TFP Redirected T Cells

The functional abilities of TFP T cells to produce cell-surface expressed TFPs, and to kill target tumor cells, proliferate and secrete cytokines can be determined using assays known in the art.

Human peripheral blood mononuclear cells (PBMCs, e.g., blood from a normal apheresed donor whose naïve T cells will be obtained by negative selection for T cells, CD4+ and CD8+ lymphocytes) will be treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays will be used to confirm cell surface presence of a TFP, such as by an anti-FLAG antibody or an anti-murine variable domain antibody. Cytokine (e.g., IFN-γ) production will be measured using ELISA or other assays.

### Example 3: Production of Anti-IL13Rα2 Nanobodies

### Library Construction

### Immunization

A llama was subcutaneously injected on days 0, 7, 14, 21, 28 and 35, each time with about 150 μg recombinant human IL13Rα2 fused to an Fc domain of human IgG1 (hIL13Rα2-Fc) (R&D Systems). The adjuvant used was GERBU adjuvant P (GERBU Biotechnik GmbH). On day 40, about 100 ml anticoagulated blood was collected from the llama for lymphocyte preparation.

### Construction of a VHH Library

A V_{HH} library was constructed from the llama lymphocytes to screen for the presence of antigen-specific nanobodies. To this end, total RNA from peripheral blood lymphocytes was used as template for first strand cDNA synthesis with an oligo(dT) primer. Using this cDNA, the V_{HH} encoding sequences were amplified by PCR, digested with PstI and NotI, and cloned into the PstI and NotI sites of the phagemid vector pMECS. The V_{HH} library thus obtained was called Core 94. The library consists of about 7×10⁸ independent transformants, with 100% of transformants harboring the vector with the right insert size.

### Isolation of Human IL13Rα2-Specific Nanobodies

The Core 94 library was panned for 3 rounds on solid-phase coated (100 μg/ml in 100 mM NaHCO₃ pH 8.2) hIL13Rα2 antigen: hIL13Rα2-Fc antigen subjected to Fc removal by Factor Xa. The binding of phages to any remaining human IgG1 Fc on the antigen coated on the well and any contaminating Factor Xa was competed by recombinant human IgG1 Fc (R&D Systems, Cat. No. 110-HG) and Factor Xa, each at a final concentration of 1 μM. The enrichment for antigen-specific phages was assessed after each round of panning by comparing the number of phagemid particles eluted from antigen-coated wells with the number of phagemid particles eluted from negative control (uncoated blocked) wells. These experiments suggested that the phage population was enriched for antigen-specific phages about 7-fold, 200-fold and 1000-fold after the 1^{st}, 2^{nd} and 3^{rd} round, respectively. In total, 190 colonies (95 from round 2 and 95 from round 3) were randomly selected and analyzed by ELISA for the presence of antigen-specific Nanobodies in their periplasmic extracts (ELISA using crude periplasmic extracts including soluble Nanobodies). The antigen used for ELISA screening was the same as the one used for panning, using uncoated blocked wells and wells coated with a mix of recombinant human IgG1 Fc and Factor Xa as negative controls. The secondary antibody (anti-mouse antibody) gave a slight background signal on the wells coated with recombinant human IgG1 Fc/Xa (about 0.3 OD at 405 nm) which labels 5 clones as cross-reactive to Fc. Out of these 190 colonies, 141 colonies scored positive for hIL13Rα2 but not for hIgG1 Fc/factor Xa mix in this assay. Based on sequence data of the 141 colonies positive on hIL13Rα2, but not on hIgG1 Fc/factor Xa mix, 54 different full length nanobodies were distinguished, belonging to 16 different CDR3 groups (B-cell lineages). Nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different. Also tested, by ELISA, was the binding of the human IL13Rα2-specific nanobodies to His-tagged human IL13Rα1 (Acro Biosystems, Cat No. IL1-H5224). These ELISA experiments revealed that none of the IL13Rα2-specific nanobodies bind human IL13Rα1. Clones from these pannings bear the following code in their names: TIG.

Flow Cytometry Analysis of hIL13Rα2-Specific Nanobodies

### Nanobodies and Cells

Periplasmic extracts were generated for each anti-hIL13Rα2 Nb in the same way as was done for the initial ELISA screening described above. Cells from each cell-line (U251_Luc_Mch and A431_Luc) were thawed, washed and counted. The periplasmic extract from each Nb clone was incubated with about 2×10⁵ cells. After washing, the cells were incubated with a mix of mouse anti-HA tag antibody and anti-mouse-PE. After another wash, Topro was added to each sample as live/dead stain and the cells were analyzed on a flow cytometer. As a positive control Mab, PE coupled anti-IL13Rα2 clone 47 (+Topro) was used. Negative controls for each cell line were: a sample with an irrelevant Nb (BCII10—bacterial β lactamase specific), a sample with all detection Mabs, a sample with the secondary anti-mouse-PE Mab alone and a sample with cells alone (with and without Topro).

### Humanization of Antibodies

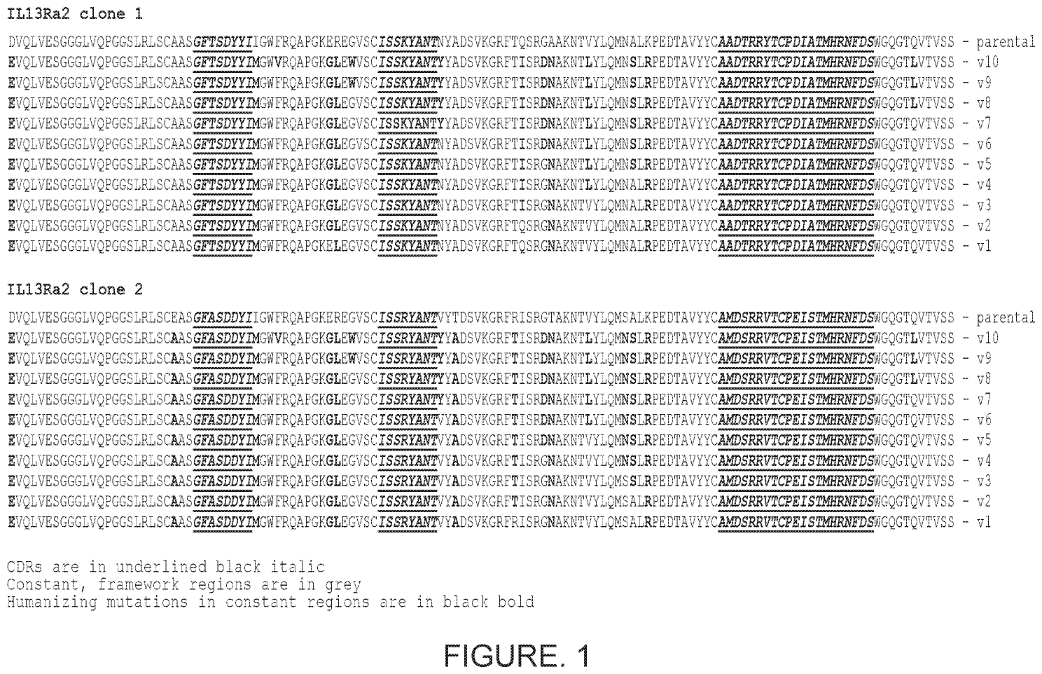

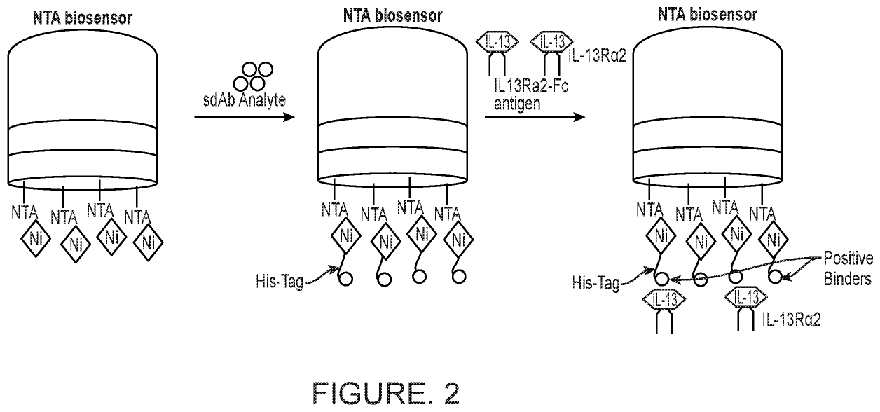

Two clones were chosen for humanization. FIG. 1 shows sequence alignments of clone 1 and clone 2, comprising the parental (non-humanized) sequence for each and ten humanized variants. Each humanized nanobody was analyzed by Octet at 500 nM on an Ni-NTA sensor, with three fold dilutions of antigen (IL13Rα2-Fc) at 125 nM, 41.66 nM, and 13.86 nM. A drawing of the experimental procedure is shown in FIG. 2. A summary of the octed measurements for each of the humanized variants depicted in FIG. 1 is shown in Table 1 (clone 1) and Table 2 (clone 2).

TABLE 1
Clone 1 parental and humanized variant analysis

| | | | Flow | | |
|---|---|---|---|---|---|
| | | | Cytometry | Octet | KD fold |
| Construct | # Mutations | % Human | MFI | KD nM | difference |
| parental | 0 | 84.62 | 2265.3 | 0.63 | 1 |
| 1-h1 | 5 | 90.11 | Not Tested | 0.42 | 0.67 |
| 1-h2 | 6 | 91.21 | Not Tested | 1.01 | 1.6 |
| 1-h3 | 7 | 92.31 | 1988.9 | 1.76 | 2.8 |
| 1-h4 | 8 | 93.41 | 1570.7 | 1.77 | 2.8 |
| 1-h5 | 9 | 94.51 | 1729.3 | 1.94 | 3.1 |
| 1-h6 | 10 | 96.70 | 1503.6 | 1.44 | 2.3 |
| 1-h7 | 11 | 96.70 | 1227.9 | 1.50 | 2.4 |
| 1-h8 | 12 | 97.80 | 1240.2 | 1.94 | 3.1 |
| 1-h9 | 13 | 98.90 | 842.1 | 1.91 | 3.0 |
| 1-h10 | 14 | 100 | 639.2 | 1.46 | 2.3 |
| 2o Ab | | | 90.4 | | |
| Isotype | | | 21.4 | | |
| Control | | | | | |

TABLE 2
Clone 1 parental and humanized variant analysis

| | | | Flow | | |
|---|---|---|---|---|---|
| | | | Cytometry | Octet | KD fold |
| Construct | # Mutations | % Human | MFI | KD uM | difference |
| 2-parental | 0 | 81.32 | 3647.4 | 0.81 | 1 |
| 2-h1 | 8 | 90.11 | Not Tested | 0.93 | 1.1 |
| 2-h2 | 9 | 91.21 | Not Tested | 3.30 | 4.1 |
| 2-h3 | 10 | 92.31 | 3065.3 | 0.91 | 1.1 |
| 2-h4 | 11 | 93.41 | 3058.4 | 0.96 | 1.2 |
| 2-h5 | 12 | 94.51 | 1453.2 | 0.87 | 1.1 |
| 2-h6 | 13 | 95.60 | 2868.7 | 1.13 | 1.4 |
| 2-h7 | 14 | 96.70 | 1903.5 | 0.95 | 1.2 |
| 2-h8 | 15 | 97.80 | 2024.2 | 0.95 | 1.2 |
| 2-h9 | 16 | 98.90 | 1637.3 | 0.87 | 1.1 |
| 2-h10 | 17 | 100 | 1122.3 | 3.31 | 4.1 |

Two humanized sequences for each clone were chosen for further study, and correspond to SEQ ID NOS:19-28 and 35-43, respectively.

### Example 4: In Vitro Activity of Anti-IL13Rα2 Nanobodies

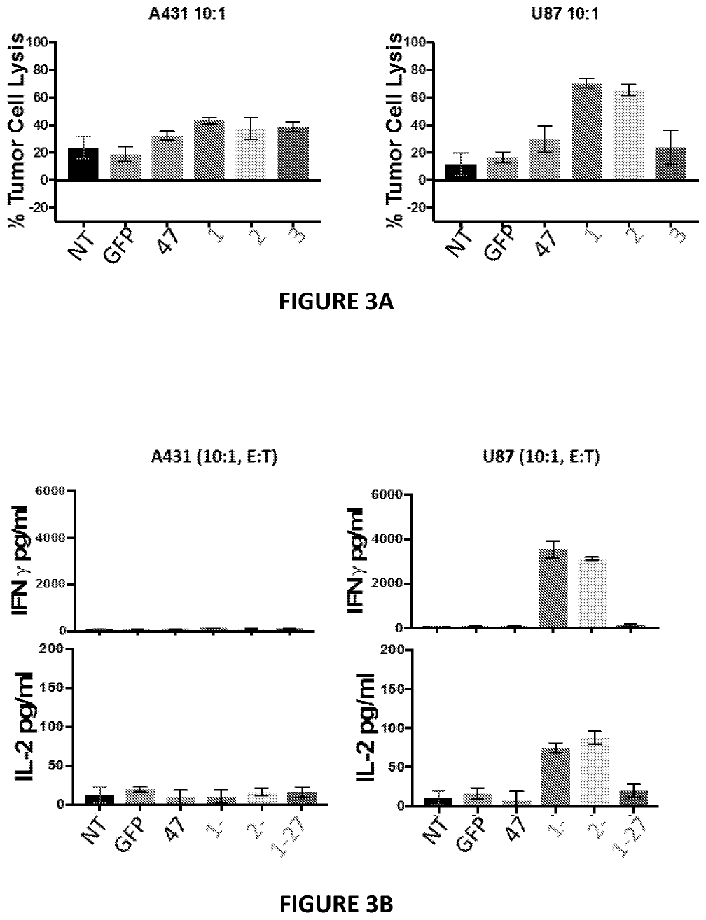

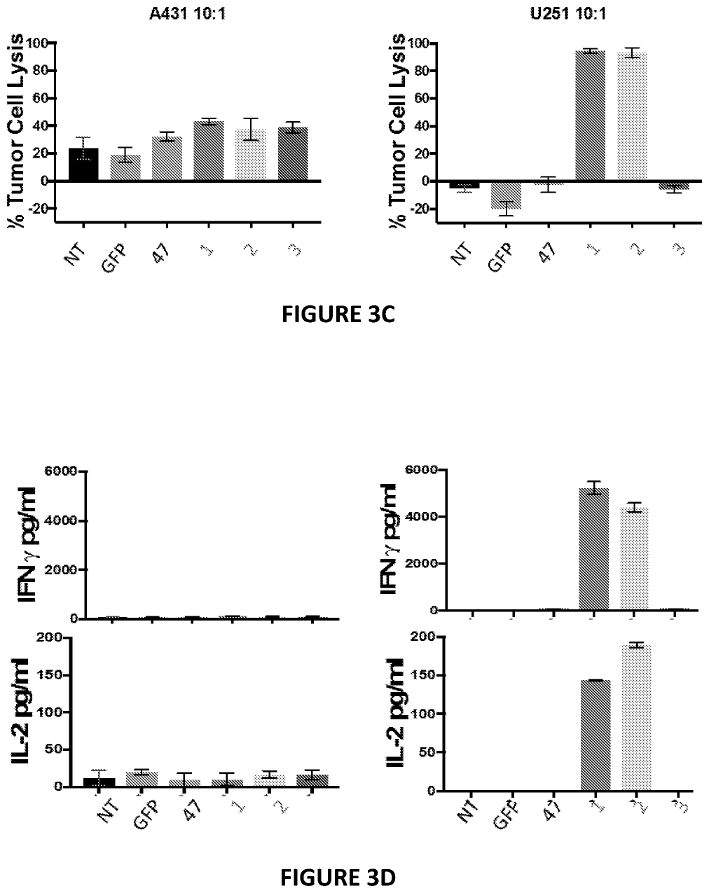

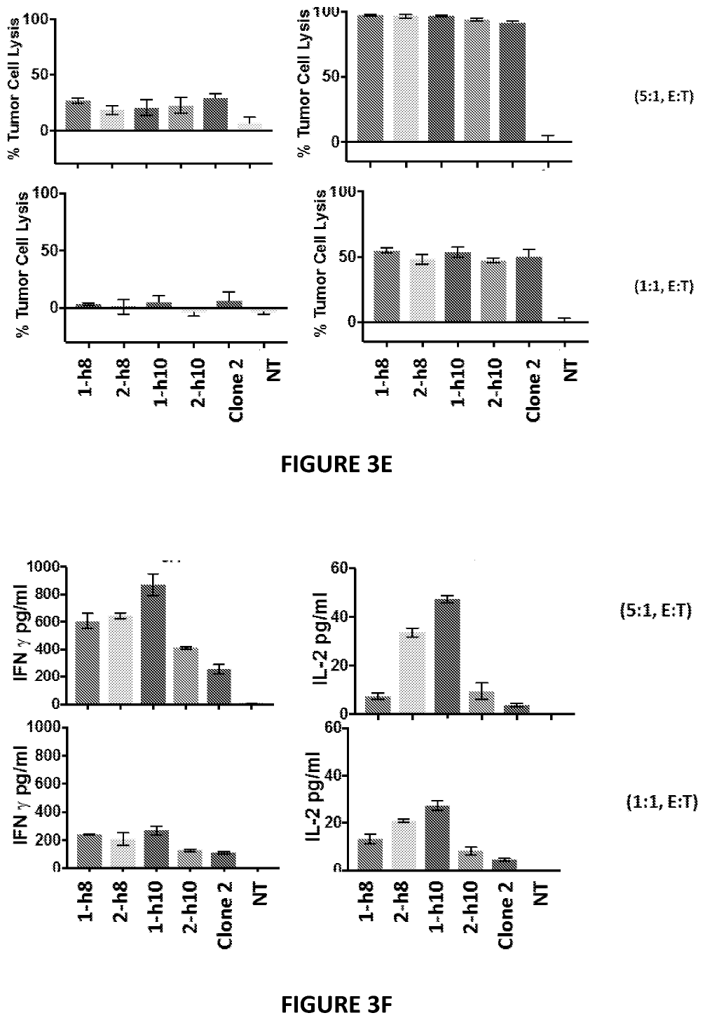

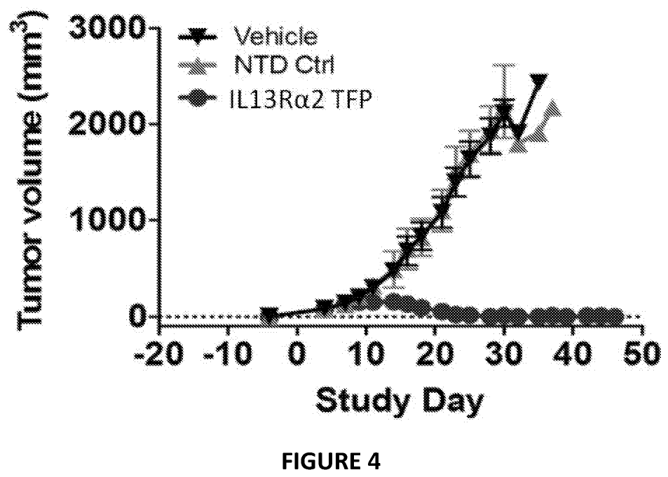

The humanized sdAbs described in Example 3 were expressed on a pLRPO backbone and incorporated into a CD3ε TFP. The corresponding IL13Rα2-TFP T cells' activity was tested on an IL-13-expressing cell line (U87) and an IL13Rα2-negative cell line (A431). Both clone 1 and clone 2 TFP T cells induced tumor cell lysis in the U87, but not A431, cells (FIG. 3A).

The same TFP T cells were tested for their ability to induce IFNγ and IL-2 production. As shown in FIG. 3B, the TFP T cells did not induce IFNγ or IL-2 from IL13Rα2 negative cells (A431) but clone 1 and clone 2 TFP T cells elicited an IFNγ response of greater than 3000 μg/ml and low IL-2 production of about 100 μg/ml. Similar results were seen when repeated in U251 glioblastoma cells.

### Example 5: Generation and Identification of Nanobodies Specific for Human MUC16 Peptide: NFSPLARRVDRVAIYEEFLRMTRNGTOLONFTLDRSSVLVDGYSPNRNEPLTGNSD LP

### Materials and Methods

### Transformation, Recloning and Expression of V_{HH}s

Transformation of Non-Suppressor Strain (e.g. WK6) with Recombinant pMECS GG

The nanobody gene cloned in pMECS GG vector contains PelB signal sequence at the N-terminus and HA tag and His₆ tag at the C-terminus (PelB leader-nanobody-HA-His₆). The PelB leader sequence directs the nanobody to the periplasmic space of the E. coli and the HA and His₆ tags can be used for the purification and detection of nanobody (e.g. in ELISA, Western Blot, etc.).

In pMECS GG vector, the His₆ tag is followed by an amber stop codon (TAG) and this amber stop codon is followed by gene III of M13 phage. In suppressor E. coli strains (e.g. TG1), the amber stop codon is read as glutamine and therefore the nanobody is expressed as fusion protein with protein III of the phage which allows the display of nanobody on the phage coat for panning. In non-suppressor E. coli strains (e.g., WK6), the amber stop codon is read as stop codon and therefore the resulting nanobody is not fused to protein III.

In order to express and purify nanobodies cloned in pMECS GG vector, simply prepare pMECS GG containing the gene of the nanobody of interest and transform a non-suppressor strain (e.g., WK6) with this plasmid. Sequence the nanobody of the resulting clone using MP057 primer (5′-TTATGCTTCCGGCTCGTATG-3′) to verify the identity of the clone. Retest antigen binding capacity by ELISA or any other appropriate assay. Now, the non-suppressor strain (e.g., WK6) containing the recombinant pMECS GG vector with the nanobody gene can be used for the expression and purification of nanobody.

Recloning Nanobody Genes from pMECS GG to pHEN6c Vector

### Primer Sequences:

Primer A6E
(5′ GAT GTG CAG CTG CAG GAG TCT GGR GGA GG 3′).
Primer PMCF
(5′ CTA GTG CGG CCG CTG AGG AGA CGG TGA CCT GGG
T 3′).
Universal reverse primer
(5′ TCA CAC AGG AAA CAG CTA TGA C 3′).
Universal forward primer
(5 CGC CAG GGT TTT CCC AGT CAC GAC 3′).

The nanobody gene is amplified by PCR using E. coli containing recombinant pMECS GG harboring the nanobody gene as template and primers A6E and PMCF (About 30 cycles of PCR, each cycle consisting of 30 seconds at 94° C., 30 seconds at 55° C. and 45 seconds at 72° C., followed by 10 minutes extension at 72° C. at the end of PCR). A fragment of about 400 bp is amplified. The PCR product is then purified (e.g. by QiaqQuick PCR purification kit from Qiagen) and digested overnight with PstI.

The PCR product is purified and digested with BstEII overnight (or with Eco91I from Fermentas) The PCR product is purified as above and the pHEN6c vector is digested with PstI for 3 hours; the digested vector is purified as above and then digested with BstEII for 2 to 3 hours The digested vector is run on a 1% agarose gel, the vector band cut out of gel and purified (e.g. by QiaQuick gel extraction kit from Qiagen). The PCR product and the vector are ligated. Electrocompetent WK6 cells are transformed with the ligation reaction. Transformants are selected using LB/agar/ampicillin (100 μg/ml)/glucose (1-2%) plates.

### Expression and Purification of Nanobodies:

A freshly transformed WK6 colony is used to inoculate 10-20 ml of LB+ampicillin (100 μg/ml)+glucose (1%) and incubated at 37° C. overnight with shaking at 200-250 rpm. 1 ml of this pre-culture is added to 330 ml TB medium supplemented with 100 μg/ml Ampicillin, 2 mM MgCl₂ and 0.1% glucose and grow at 37° C. with shaking (200-250 rpm) until an OD₆₀₀ of 0.6-0.9 is reached. Nanobody expression is induced by addition of IPTG to final concentration of 1 mM and the culture is incubated at 28° C. with shaking overnight (about 16-18 hours; the OD₆₀₀ after overnight induction should ideally be between 25 and 30). The culture is centrifuged for 8 minutes at 8000 rpm and the pellet resuspended from 1 liter culture in 12 ml TES and shaken for 1 hour on ice. Per each 12 ml TES used, 18 ml TES/4 is added and further incubated on ice for an additional hour (with shaking) and then centrifuged for 30 min at 8000 rpm at 4° C. The supernatant contains proteins extracted from the periplasmic space.

### Purification by IMAC:

His-select is equilibrated with PBS: per periplasmic extract derived from 1 liter culture, 1 ml Resin is added (about 2 ml His-select solution) to a 50 ml falcon tube, PBS is added to a final volume of 50 ml and mixed and then centrifuged at 2000 rpm for 2 min. and the supernatant discarded. The resin is washed twice with PBS and then the periplasmic extract is added and incubated for 30 minutes to 1 hour at room temperature with gentle shaking (longer incubation times may result in non-specific binding). The sample is loaded onto a PD-10 column with a filter at the bottom (GE healthcare, cat. No. 17-0435-01) and washed with 50 to 100 ml PBS (50-100 ml PBS per 1 ml resin used). Elution is performed 3 times, each time with 1 ml PBS/0.5 M imidazole per 1 ml resin used, and dialyzed overnight at 4° C. against PBS (cutoff 3500 daltons) to remove imidazole.

The amount of protein can be estimated at this point by OD₂₈₀ measurement of eluted sample. Extinction coefficient of each clone can be determined by protParam tool under primary structure analysis at the Expasy proteomics server. Further purification of nanobodies can be achieved by different methods. For example, the sample may be concentrated (Vivaspin 5000 MW cutoff, Vivascience) by centrifuging at 2000 rpm at 4° C. till an appropriate volume for loading on a Superdex 75 16/60 is obtained (max. 4 ml). The concentrated sample is then loaded onto a Superdex 75 16/60 column equilibrated with PBS. Peak fractions are pooled and the sample is measured at OD₂₈₀ for quantification. Aliquots are stored at −20° C. at a concentration of about 1 mg/ml.

### Immunization

A llama was subcutaneously injected on days 0, 7, 14, 21, 28 and 35, with human MUC16 peptide (hMUC16) conjugated to KLH (NFSPLARRVDRVAIYEEFLRMTRNGTQLQNFTLDRSSVLVDGYSPNRNEPLTGNSDLP—C-KLH) and/or human MUC16 peptide biotinylated at C-terminus (NFSPLARRVDRVAIYEEFLRMTRNGTQLQNFTLDRSSVLVDGYSPNRNEPLTGNSDLP—C-Biotin) and/or human MUC16 peptide biotinylated at N-terminus (Biotin—NFSPLARRVDRVAIYEEFLRMTRNGTQLQNFTLDRSSVLVDGYSPNRNEPLTGNSDLP. The biotinylated peptides were mixed with neutralite avidin before injections. The adjuvant used was GERBU adjuvant P (GERBU Biotechnik GmbH. On day 40, about 100 ml anticoagulated blood was collected from the llama for lymphocyte preparation.

### Construction of a VHH Library

A VHH library was constructed from the llama lymphocytes to screen for the presence of antigen-specific nanobodies. To this end, total RNA from peripheral blood lymphocytes was used as template for first strand cDNA synthesis with an oligo(dT) primer. Using this cDNA, the VHH encoding sequences were amplified by PCR, digested with SAPI, and cloned into the SAPI sites of the phagemid vector pMECS-GG. The VHH library thus obtained was called Core 93GG. The library consisted of about 10⁸ independent transformants, with about 87% of transformants harboring the vector with the right insert size.

### Isolation of Human MUC16 Peptide-Specific Nanobodies

The Core 93GG library was panned on hMUC16 peptide NFSPLARRVDRVAIYEEFLRMTRNGTQLQNFTLDRSSVLVDGYSPNRNEPLTGNSDLP biotinylated either at C- or N-terminus (bio-hMUC16) for 4 rounds. The bio-hMUC16 peptide was allowed to interact with streptavidin coated plates after which phages from the library were added to the plate. The enrichment for antigen-specific phages was assessed after each round of panning by comparing the number of phagemid particles eluted from antigen-coated wells with the number of phagemid particles eluted from negative control wells (coated with streptavidin and blocked but containing no peptide). These experiments suggested that the phage population was enriched for antigen-specific phages about 2-fold after the 2^{nd} round. No enrichment was observed after the 1^{st}, 3^{rd} and 4^{th} round. In total, 380 colonies (190 from round 3, 190 from round 4) were randomly selected and analyzed by ELISA for the presence of antigen-specific nanobodies in their periplasmic extracts (ELISA using crude periplasmic extracts including soluble nanobodies). The peptides used for ELISA screening were the same as the ones used for panning, using blocked streptavidin-coated wells without peptide as negative control. Out of these 380 colonies, 34 colonies scored positive in this assay. Based on sequence data of the positive colonies, 6 different full length nanobodies were distinguished, belonging to 2 different CDR3 groups (B-cell lineages) (see Excel file). Nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different. Clones from these pannings bear the following code in their name: MU.

Flow Cytometry Analysis of hMUC16 Peptide-Specific Nanobodies

### Nanobodies and Cells

Periplasmic extracts were generated for each anti-hMUC16-peptide Nb in the same way as was done for the initial ELISA screening described above. Cells from each cell-line (SKOV3 Muc16 Luc, OVCAR 3 Muc16 Luc, Expi-293 and Jurkat) were thawed, washed and counted. The periplasmic extract from each Nb clone was incubated with about 2×10⁵ cells. After washing, the cells were incubated with a mix of mouse anti-HA tag antibody and anti-mouse-PE. After another wash, Topro was added to each sample as live/dead stain and the cells were analyzed on a flow cytometer. As a positive control Mab, human anti-Muc16-4h11 (+anti-human IgG-PE+Topro), was used on the SKOV3 Muc16 Luc and OVCAR 3 Muc16 Luc cells. As negative controls, we used for each cell line: a sample with an irrelevant Nb (BCII10—bacterial β lactamase specific), a sample with all detection Mabs, a sample with the secondary anti-mouse-PE Mab alone and a sample with cells alone (with and without Topro).

### Example 6: Screening of Anti-MUC16 sdAbs to the hMUC16 Target

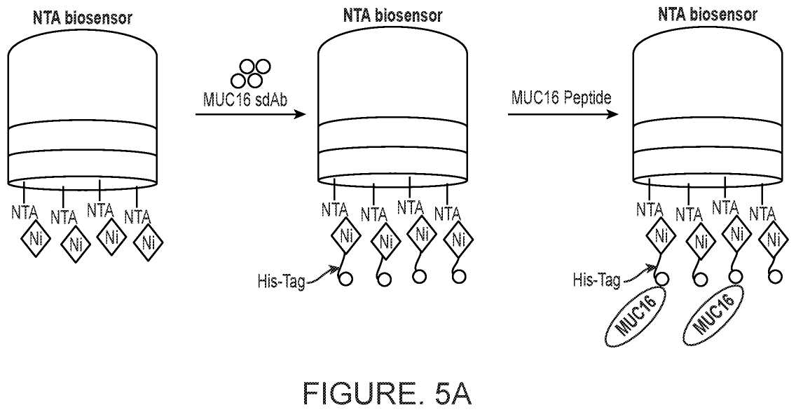

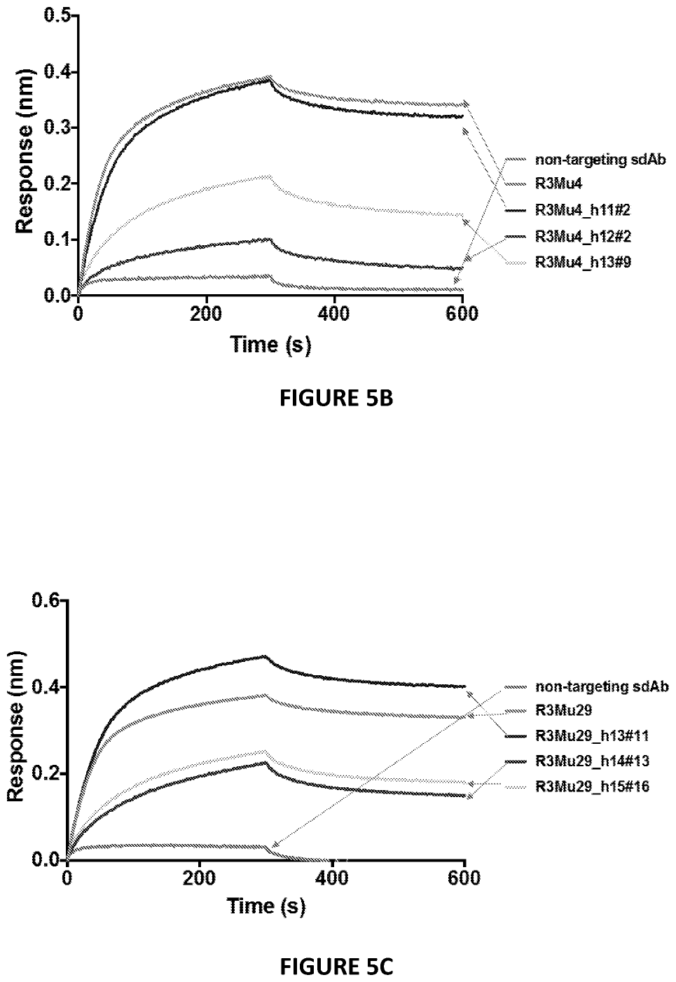

The V_{HH} binders produced in Example 5 are screened using an NTA biosensor (nickel column, see FIG. 5A for a drawing outlining the method). The His-tagged MUC16 sdAbs (3.25 μg/ml) are bound to the column, and then the MUC16 peptide is passed through the column at concentrations of 200, 100, 50, 25, 6.25, 1.56, and 0 nM. Buffer: 1× Corning® Cellgro® PBS pH 7.4 (cat. 21-040-CM) containing 0.02% Tween® 20) at 30° C. Sensors: Pall Forte Bio Dip & Read (cat. 18-5102).

Saturation binding of two clones, R3Mu4 (FIG. 1C) and R3Mu29 (FIG. 1D) llama and humanized sdAbs to the MUC16 target demonstrate that parental and humanized αMuc16 sdAb variants exhibit high affinity binding to MUC16 ectodomain (“MUC16^{ecto}”) peptide, associated with values of K_{D} ranging from 6-94 nM. There is some loss in affinity demonstrated by humanized variants compared to their respective parental llama clones. A summary is provided in Table 3.

TABLE 3
Determination of K_{D} from 1:1 global fit model of titration binding

| αMu16 sdAb* | K_{D} (nM) |
|---|---|
| Parental R3Mu4 | 10.2 ± 1.7 |
| R3Mu4 h11#2 | 10.9 ± 1.6 |
| R3Mu4 h12#2 | 94.6 ± 1.5 |
| R3Mu4 h13#9 | 36.2 ± 15.4 |
| Parental R3Mu29 | 6.3 ± 0.1 |
| R3Mu29_h13#11 | 8.3 ± 0.1 |
| R3Mu29_h14#13 | 36.4 ± 0.8 |
| R3Mu29_h15#16 | 22.9 ± 0.4 |

### Example 7: Epitope Binning of Anti-MUC16 Binders in Comparison with 4H11 Tool Binder

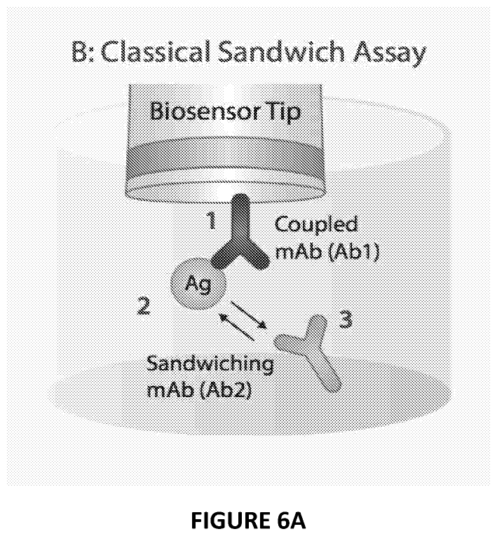

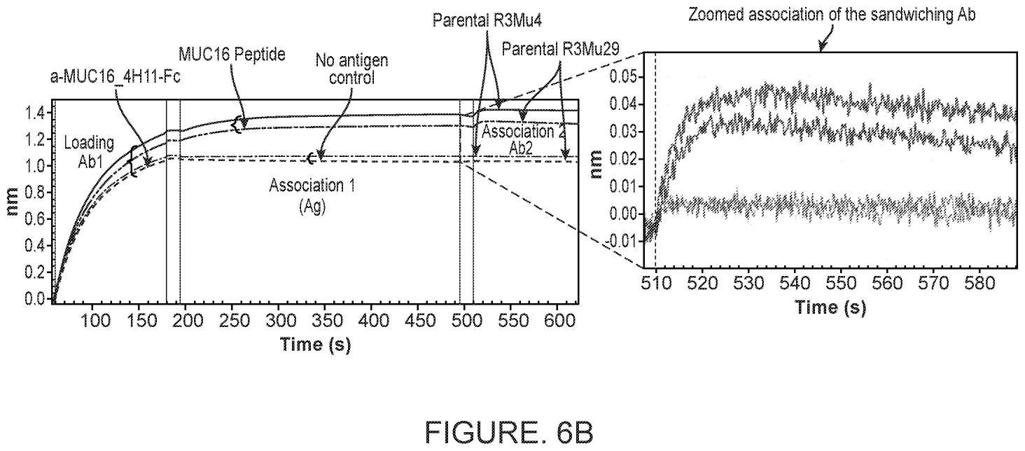

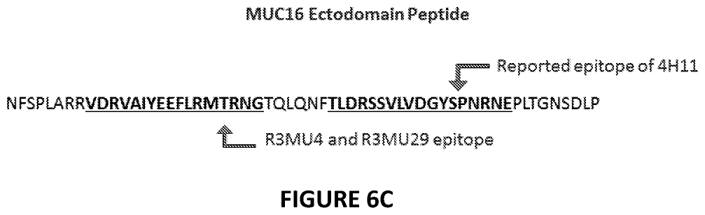

To determine if the MUC16 parental R3Mu4 and parental R3Mu29 sdAbs bin to same or different epitopes compared to the 4H11 scFv-Fc tool binder (from 4H11 hybridoma), a sandwich assay was used (See FIG. 6A).

As shown in FIG. 6B, the MUC 16 sdAbs—parental (llama) R3Mu4 and parental (llama) R3Mu29 show binding following 4H11 tool binder exposure, demonstrating that the parental sdAbs recognize and bin to a different epitope of MUC16 peptide as compared to 4H11 scFv-Fc tool binder. The negative control with no antigen (MUC16 peptide) shows no binding, ruling out any chances of non-specific binding. A diagram showing the binding epitope of the parental llama antibodies R3Mu4 and R3Mu29 is shown in FIG. 6C.

### Example 8: Preclinical Studies with T Cells Expressing MUC16-TFP

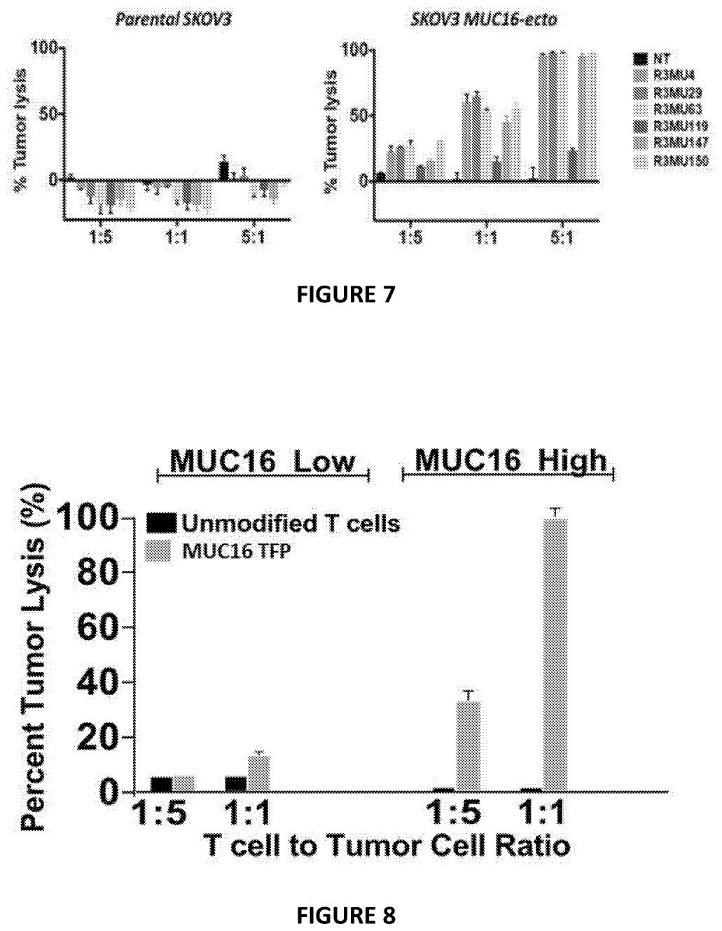

T cells expressing MUC16-TFPs were evaluated in preclinical in vitro studies (FIG. 7). T cells expressing MUC16-TFPs specifically killed SKOV3-MUC16Cterm ovarian cancer cells that were transduced to overexpress the C-terminal cell associated MUC16 form in a dose-dependent manner, while the parental SKOV3 MUC16-negative cells were spared from T cells expressing MUC16-TFPs mediated killing. Likewise, T cells expressing MUC16-TFPs eliminated OVCAR3-MUC16-Cterm cells that overexpressed the cell-associated form of MUC16. Parental OVCAR3 cells expressing low levels of MUC16 were only killed at the highest TFP-T cell-to-target cell ratio, which underscores the dose-dependent lysis of tumor cells. Likewise, TFP-T cells only released cytokines when MUC16 was present on the target cells. FIG. 8 depicts example experimental data showing the potency of MUC16-TFP in cellular assays using ovarian cell lines expressing high and low levels of MUC16. In these studies, MUC16-TFP was observed to have preferential killing abilities depending on the level of MUC16 on the tumor cell surface. More precisely, MUC16-TFP was observed to kill high MUC16 expressing tumor cells in a dose dependent fashion, whereas MUC16-TFP killing of low MUC16 expressing cells was not observed at the dose levels used in these assays.

### Example 9. Flow Cytometry Based MUC16^{ecto} Copy Number Quantitation

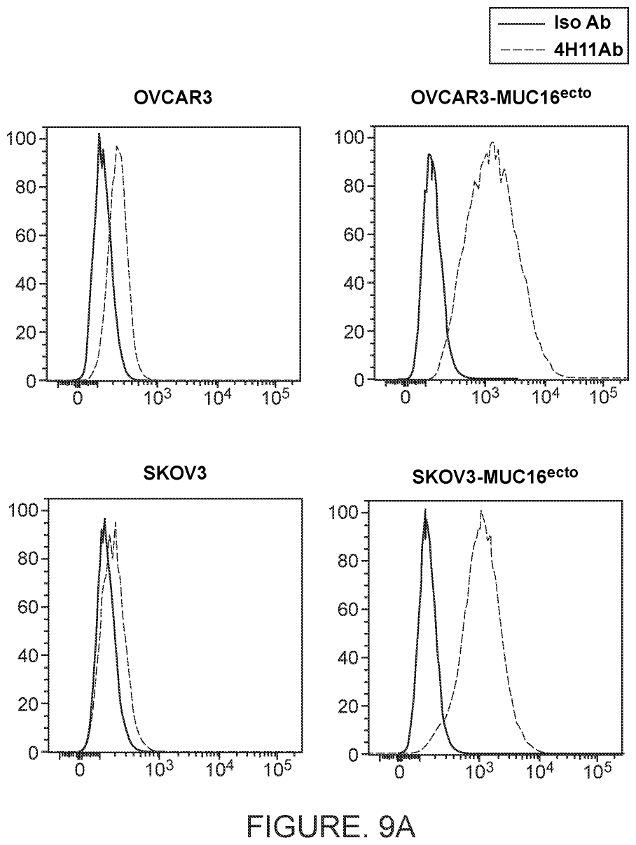

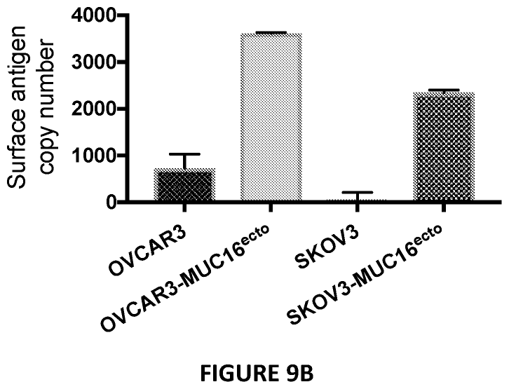

C-terminal cell associated MUC16 form (MUC16^{ecto}) specific antibody 4H11 was produced according to U.S. Pat. No. 9,169,328 and then conjugated with PE. The average number of PE molecules per antibody was estimated to be about 1. Ovarian carcinoma cell lines OVCAR3 and SKOV3, or the derivatives stably overexpressing MUC16^{ecto} (OVCAR3-MUC16^{ecto} and SKOV3-MUC16^{ecto} cells), were stained with the 4H11-PE Ab at 2 μg per sample. The copy number of cell-surface MUC16^{ecto} was estimated by Quantibrite Beads PE Fluorescence Quantitation kit (BD Bioscience) per manufacture's instruction. 4H11-PE antibody-stained tumor cells were run on Fortessa® X-20 together with the Quantibrite beads. The geometric median fluorescent intensity (gMFI) was calculated for the cells as well as the beads. The beads stock contains 4 populations manufactured to have different number of PE molecules per bead (high, moderate, low, negative). A standard curve was generated based on the given copies of PE molecules per bead versus the measured MFI for each set of beads. The copy number of MUC16^{ecto} on tumor cells were then estimated based on the beads-generated standard curve. The copies of MUC16^{ecto} on OVCAR3, OVCAR3-MUC16^{ecto}, SKOV3 and SKOV3-MUC16^{ecto} cells were determined as 726, 3616, 39 and 2351, respectively (FIG. 9B).

### Example 10. MUC16^{ecto} Specific Tumor Cell Lysis by MUC16-TFP T Cells

MUC16^{ecto} specific tumor cell lysis by MUC16-TFP T cells were evaluated by in vitro cytotoxicity assay. Tumor cell lines with or without MUC16^{ecto} expression were stably transduced to express firefly luciferase as the reporter. After twenty-four hours co-culture, the luciferase activity of the co-cultured cells was determined, with Bright-Glom Luciferase Assay System (Promega, Cat #E2610), as surgate of residual alive tumor cells. The percentage of tumor cell killing was then calculated with the following formula: % of Tumor Cell Lysis=100%×[1−RLU (Tumor cells+ T cells)/RLU (Tumor cells)].

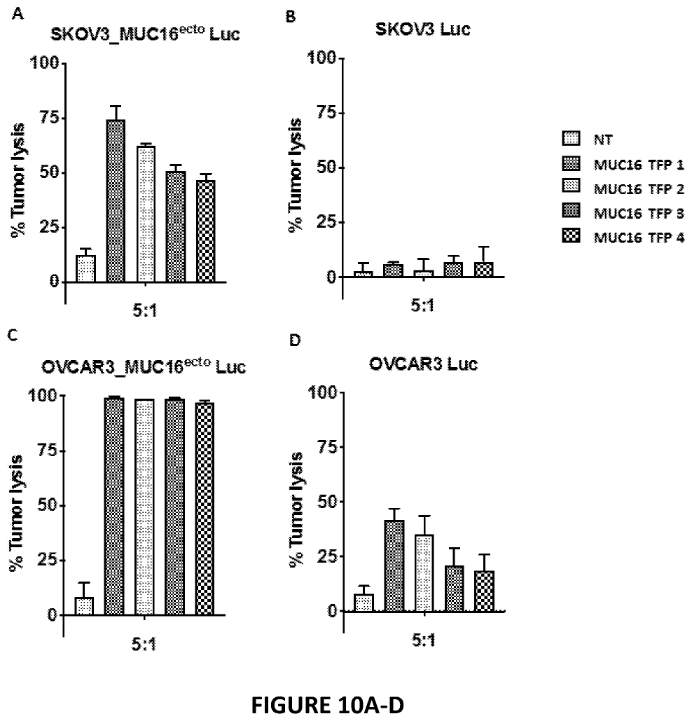

T cells expressing MUC16-TFPs specifically killed SKOV3-MUC16^{ecto} cells (FIG. 10A), while the parental SKOV3 cells were spared from T cells expressing MUC16-TFPs mediated killing (FIG. 10B). Likewise, T cells expressing MUC16-TFPs eliminated OVCAR3-MUC16^{ecto} cells that overexpressed the cell-associated form of MUC16 (FIG. 10C). Parental OVCAR3 cells expressing low levels of MUC16^{ecto} were only killed partially (FIG. 10D).

### Example 11. MUC16_{ecto} Specific Cytokine Production by MUC16-TFP T Cells

MUC16^{ecto} specific cytokine production by MUC16-TFP T cells were determined for the supernatant harvested from co-culture of various tumor cells, with or without MUC16^{ecto} expression and MUC16-TFP T cells. The levels of human IFN-γ and IL-2 in the supernatant were analyzed using MAGPIX Luminex® xMAP Technology (EMD Millipore), with 2-plex kits (Millipore, Catalog #HCYTOMAG-60K).

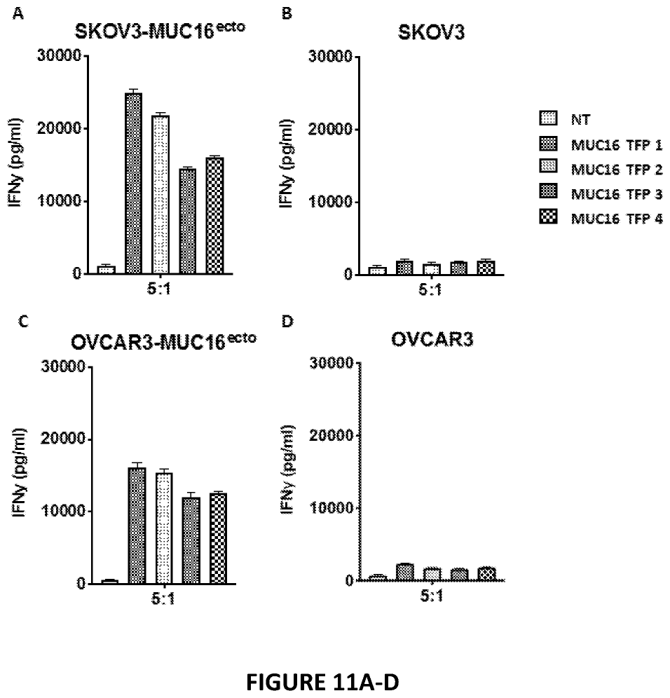

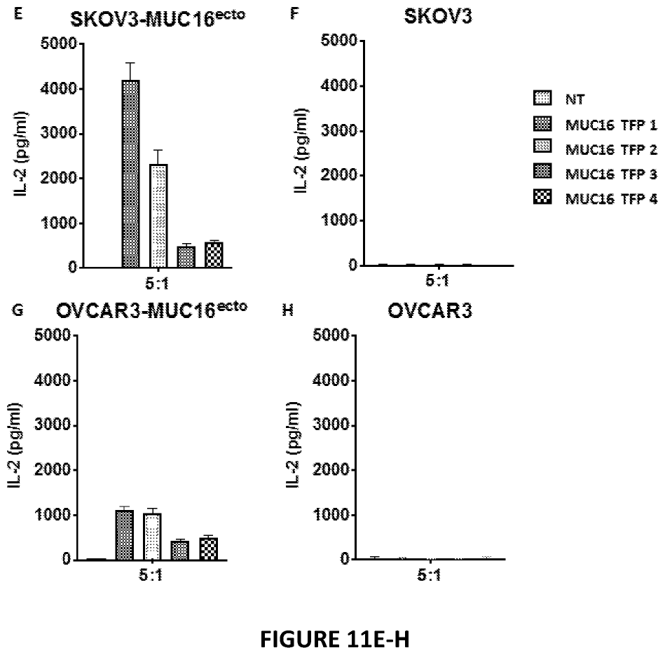

T cells expressing MUC16-TFPs secreted pro-inflammatory cytokines in an antigen-specific manner. T cells expressing MUC16-TFPs secreted IFN-γ and IL-2 when co-cultured with SKOV3-MUC16^{ecto} cells (FIGS. 11A and 11E, respectively) or OVCAR3-MUC16^{ecto} cells (FIGS. 11C and 11G, respectively), but not with SKOV3 cells (FIGS. 11B and 11F, respectively) or OVCAR3 cells (FIGS. 11D and 11H, respectively).

### Example 12. MUC16^{ecto} Specific Proliferation of T Cells Expressing MUC16-TFP

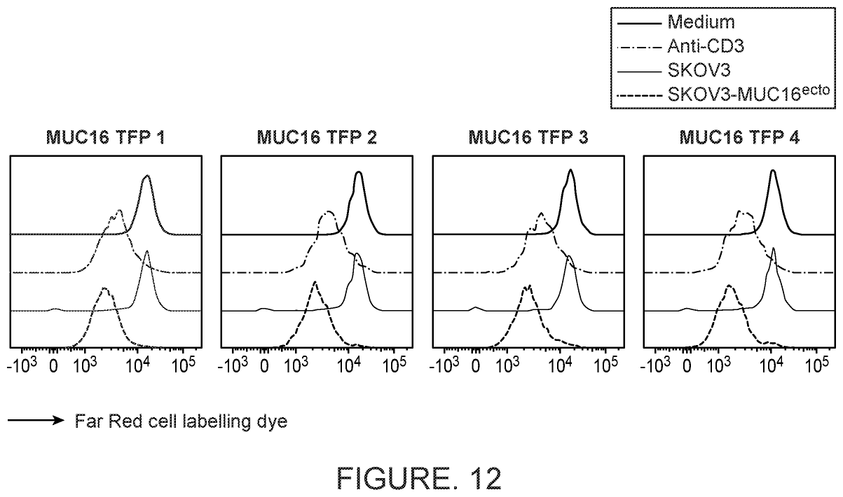

MUC16^{ecto} specific proliferation of MUC16-TFP T cells were determined by monitoring the dilution of T cell tracing signal (decrease in signal intensity of CellTrace™) by flowcytometry analysis. T cells expressing MUC16-TFPs were labelled with CellTrace™ Far Red Proliferation Kit (Cat. #C34564ThermoFisher), then co-cultured with SKOV3 or SKOV3-MUC16^{ecto} cells at 1-to-1 ratio for 3 days. T cells expressing MUC16-TFPs labelled with CellTrace Far Red Proliferation kit were also stimulated with medium alone or with 1 μg/mL plate-bound anti-CD3 antibody (clone OKT-3, Cat #14-0037-82, Invitrogen) for 3 days. T cells expressing MUC16-TFPs showed MUC16^{ecto} specific proliferation, demonstrated by the decrease of CellTracer signal when co-cultured with SKOV3-MUC16^{ecto} cells, but not SKOV3 cells (FIG. 12).

### Example 13. In Vivo Activity of MUC16-TFP T Cells

T cells expressing MUC16-TFPs were evaluated in NSG mouse xenograft models of human ovarian carcinoma cell lines, SKOV3-MUC16^{ecto} cells and OVCAR3-MUC16^{ecto} cells. Six-week-old female NSG (NOD.Cg-Prkdc^{scid}Il2rgtmiwji/SzJ, The Jackson Laboratory, stock number 005557) mice were intraperitoneally inoculated with SKOV3-MUC16^{ecto} cells (5×10⁵ cells/mouse) or OVCAR3-MUC16^{ecto} cells (5×10⁶ cells/mouse), or subcutaneously with SKOV3-MUC16^{ecto} cells (5×10⁶ cells/mouse, 1-to-1 mixture with Matrigel®). Tumor burden was determined by bioluminescence imaging (BLI) for the intraperitoneal models with the intraperitoneal injection of 0.2 ml of luciferin substrate (VWR) diluted in PBS (150 mg/kg). Tumor burden of the subcutaneous model was measured as the tumor volume by Caliper. Once the tumor model was established (intraperitoneal models: BLI signal >10⁸; subcutaneous model: tumor volume >75 mm³), T cells expressing MUC16-TFPs (MUC16 TFP1 and MUC16 TFP2) or non-transduced T cells (NT), or vehicle (PBS) were injected intravenously at the dose of 10⁷ T cells per mouse.

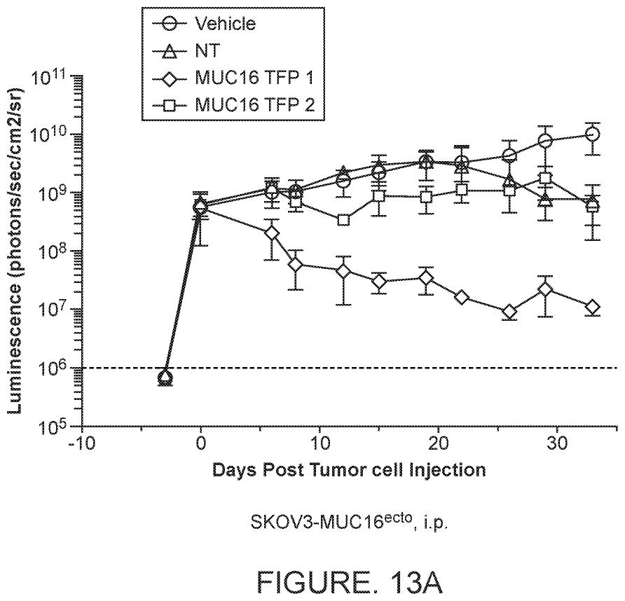

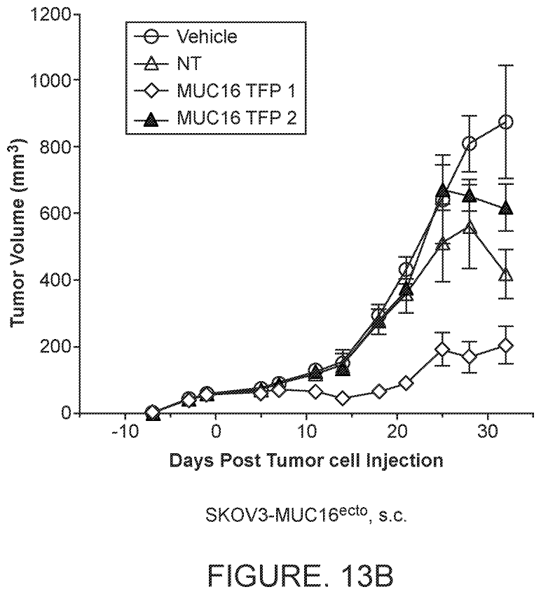

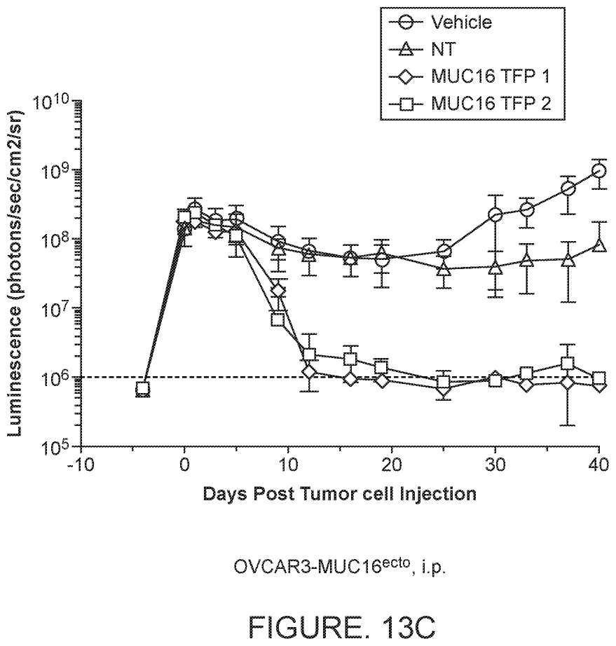

The in vivo efficacy of T cells expressing MUC16 TFPs was observed across intraperitoneal and subcutaneous models of SKOV3-MUC16^{ecto} cells and OVCAR3-MUC16^{ecto} cells. In intraperitoneal model of SKOV3-MUC16^{ecto} cells, MUC16 TFP 1 showed significant decrease of the tumor burden in comparison to the baseline level on day 0 (day of T cell injection) (FIG. 13A). Consistently, MUC16 TFP1 significantly delayed the tumor growth in subcutaneous models of SKOV3-MUC16^{ecto} cells, when compared to NT T cells (FIG. 13B). In the intraperitoneal model of OVCAR3-MUC16^{ecto} cells, MUC16 TFP1 and MUC16 TFP2 both completed cleared tumor from the mice (FIG. 13C).

### Example 14: Immunohistochemistry Staining of Normal Human Tissues Using Anti-MUC16 Single Domain Antibody Fc Fusion Protein

The objective of the studies was to obtain information on the MUC16 expression of normal human tissues.

Control materials and FFPE sections were stained with an anti-MUC16 single domain antibody that was genetically fused to a mouse Fc region for detection using HRP conjugated anti-mouse Fc secondary antibody. The positive control consisted of FFPE sections of human ovarian tumors from two donors. The negative control was an FFPE section of a human heart. The panel of tested tissues included the following: blood cells, cerebellum or cerebral cortex, gastrointestinal tract (esophagus, small intestine, stomach, colon—as available), spleen, kidney (glomerulus, tubule), liver, lymph node, skin, placenta, testis and tonsil from one donor each.

Results: Two human ovarian carcinoma tissues from different donors were used as a positive control and showed staining at different intensities, ranging from 1-3+(occasional to frequent) and 1-4+(occasional to frequent) for neoplastic cell membranes and cytoplasm. From the normal tissues, all showed negative staining for MUC16 but two: 1) human stomach epithelium, parietal (cytoplasm, cytoplasmic granules)—1-2+(occasional to frequent), and 2) human tonsil epithelium surface, crypt (membrane, cytoplasm and other elements)—1-3+ rare to occasional.

These data demonstrate that MUC16 has limited expression in normal human tissues and elevated expression in certain tumors. This makes it an attractive target for cancer therapy of MUC16 positive malignancies. The MUC16-specific single domain antibody was able to bind and stain antigen positive tissues.

### Example 15: Clinical Studies

Patients with unresectable ovarian cancer with relapsed or refractory disease will be enrolled for clinical studies of T cells expressing MUC16 TFPs. The initial study will explore the safety profile of T cells expressing MUC16 TFPs and will explore cell kinetics and pharmacodynamics outcomes. Those results will inform the selection of dosages for further studies, which will then be administered to a larger cohort of patients with unresectable ovarian cancer to define the efficacy profile of T cells expressing MUC16 TFPs.

### Example 16: Anti-MSLN TFP T Cells Preferentially Kill Tumor Cells with High MSLN Expression

The differential killing ability of MSLN-TFP T cells against MSLN high (MSTO-MSLN^{high}, 11006 copies of surface MSLN) and MSLN low tumors (MSTO-MSLN^{low}, 198 copies surface MSLN) was addressed in NSG mouse bearing either MSTO-MSLN^{high} or MSTO-MSLNI^{low} tumors.

The MSTO-MSLN^{high} and MSTO-MSLN^{low} cells were resuspended in sterile PBS (pH 7.4) at a concentration of 1×10⁶ cells/100 μL. The PBS cell suspension was then mixed 1:1 with ice cold Matrigel® for a final injection volume of 200 μL per mouse. To all animals, 200 μL of tumor cell suspension in sterile PBS/Matrigel® was injected by subcutaneous administration in the dorsal hind flank based. Tumor growth was monitored by tumor volume, measured twice a week by caliper. Once the tumor model is established (14 days after tumor injection), with average tumor volume reaches ˜300 mm³, the tumor bearing mice were injected intravenously with non-transduced T cells (NT, 1×10⁷ total T cells) or MSLN-TFP T cells (1×10⁷ total T cells).

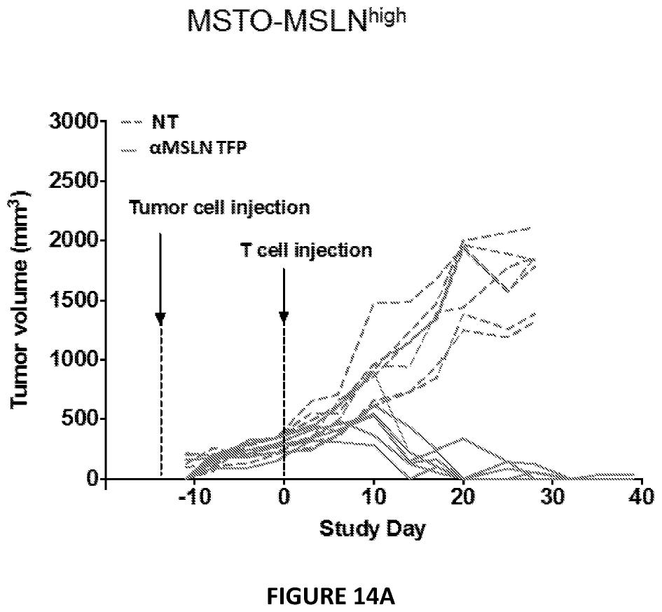

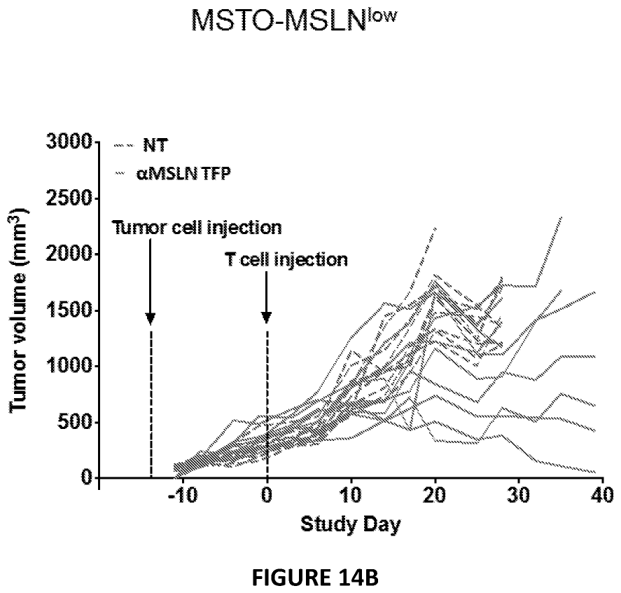

MSLN-TFP T cells dramatically controlled the growth of MSLN high tumors, compared to NT T cells treated mice (FIG. 14A). On the other hand, limited anti-tumor response were observed in MSLN-TFP T cells treated mice with MSLN low tumors (FIG. 14B). While tumor regression was observed in one animal, the other 9 MSLN-TFP T cells treated mice showed either slower (n=2) or similar (n=6) rate of tumor progression to animals receiving NT T cells (FIG. 14B).

### ENDNOTES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 4
SEQUENCES

| SEQ | | |
|---|---|---|
| ID | | |
| NO: | Name | Sequence |
| 1 | Short Linker 1 | GGGGSGGGGSGGGGSLE |
| 2 | Short Linker 2 | AAAGGGGSGGGGSGGGGSLE |
| 3 | Long Linker | AAAIEVMYPPPYLGGGGSGGGGSGGGGSLE |
| 4 | human CD3-ε | MQSGTHWRVLGLCLLSVGVWGQDGNEEMGGITQTPYKVSISGTTV |
| | | ILTCPQYPGSEILWQHNDKNIGGDEDDKNIGSDEDHLSLKEFSELEQ |
| | | SGYYVCYPRGSKPEDANFYLYLRARVCENCMEMDVMSVATMVD |
| | | ICITGGLLLLVYYWSKNRKAKAKPVTRGAGAGGRQRGQNKERPPP |
| | | VPNPDYEPIRKGQRDLYSGLNQRRI |
| 5 | human CD3-γ | MEQGKGLAVLILAIILLQGTLAQSIKGNHLVKVYDYQEDGSVLLTC |
| | | DAEAKNITWFKDGKMIGFLTEDKKKWNLGSNAKDPRGMYQCKGS |
| | | QNKSKPLQVYYRMCQNCIELNAATISGFLFAEIVSIFVLAVGVYFIA |
| | | GQDGVRQSRASDKQTLLPNDQLYQPLKDREDDQYSHLQGNQLRR |
| | | N |
| 6 | human CD3-δ | MEHSTFLSGLVLATLLSQVSPFKIPIEELEDRVFVNCNTSITWVEGTV |
| | | GTLLSDITRLDLGKRILDPRGIYRCNGTDIYKDKESTVQVHYRMCQS |
| | | CVELDPATVAGIIVTDVIATLLLALGVFCFAGHETGRLSGAADTQAL |
| | | LRNDQVYQPLRDRDDAQYSHLGGNWARNKS |
| 7 | human CD3-ζ | MKWKALFTAAILQAQLPITEAQSFGLLDPKLCYLLDGILFIYGVILT |
| | | ALFLRVKFSRSADAPAYQQGQNQLYNELNLGRREEYDVLDKRRGR |
| | | DPEMGGKPQRRKNPQEGLYNELQKDKMAEAYSEIGMKGERRRGK |
| | | GHDGLYQGLSTATKDTYDALHMQALPPR |
| 8 | human TCR α- | MAGTWLLLLLALGCPALPTGVGGTPFPSLAPPIMLLVDGKQQMVV |
| | chain | VCLVLDVAPPGLDSPIWFSAGNGSALDAFTYGPSPATDGTWTNLAH |
| | | LSLPSEELASWEPLVCHTGPGAEGHSRSTQPMHLSGEASTARTCPQE |
| | | PLRGTPGGALWLGVLRLLLFKLLLFDLLLTCSCLCDPAGPLPSPATT |
| | | TRLRALGSHRLHPATETGGREATSSPRPQPRDRRWGDTPPGRKPGS |
| | | PVWGEGSYLSSYPTCPAQAWCSRSALRAPSSSLGAFFAGDLPPPLQ |
| | | AGA |
| 9 | human TCR α- | PNIQNPDPAVYQLRDSKSSDKSVCLFTDFDSQTNVSQSKDSDVYITD |
| | chain C region | KTVLDMRSMDFKSNSAVAWSNKSDFACANAFNNSIIPEDTFFPSPES |
| | | SCDVKLVEKSFETDTNLNFQNLSVIGFRILLLKVAGFNLLMTLRLWS |
| | | S |
| 10 | human TCR α- | MAMLLGASVLILWLQPDWVNSQQKNDDQQVKQNSPSLSVQEGRIS |
| | chain V region | ILNCDYTNSMFDYFLWYKKYPAEGPTFLISISSIKDKNEDGRFTVFL |
| | CTL-L17 | NKSAKHLSLHIVPSQPGDSAVYFCAAKGAGTASKLTFGTGTRLQVT |
| | L | |
| 11 | human TCR β- | EDLNKVFPPEVAVFEPSEAEISHTQKATLVCLATGFFPDHVELSWW |
| | chain C region | VNGKEVHSGVSTDPQPLKEQPALNDSRYCLSSRLRVSATFWQNPRN |
| | | HFRCQVQFYGLSENDEWTQDRAKPVTQIVSAEAWGRADCGFTSVS |
| | | YQQGVLSATILYEILLGKATLYAVLVSALVLMAMVKRKDF |
| 12 | human TCR β- | MGTSLLCWMALCLLGADHADTGVSQNPRHNITKRGQNVTFRCDPI |
| | chain V region | SEHNRLYWYRQTLGQGPEFLTYFQNEAQLEKSRLLSDRFSAERPKG |
| | CTL-L17 | SFSTLEIQRTEQGDSAMYLCASSLAGLNQPQHFGDGTRLSIL |
| 13 | human TCR β- | MDSWTFCCVSLCILVAKHTDAGVIQSPRHEVTEMGQEVTLRCKPIS |
| | chain V region | GHNSLFWYRQTMMRGLELLIYFNNNVPIDDSGMPEDRFSAKMPNA |
| | YT35 | SFSTLKIQPSEPRDSAVYFCASSFSTCSANYGYTFGSGTRLTVV |
| 14 | Nucleic acid | caggtgcagctgcaggagtctgggggaggattggtgcaggctggggg |
| | sequence | ctctctgagactctcctgtgcagcctctggacgcaccgtcagtagct |
| | encoding single | tgttcatgggctggttccgccaagctccagggaaggagcgtgaactt |
| | domain anti- | gtagcagccattagccggtatagtctatatacatactatgcagactc |
| | MUC16 binder 1 | cgtgaagggccgattcaccatctccgcagacaacgccaagaacgcgg |
| | (SD1) | tatatctgcaaatgaacagcctgaaacctgaggacacggccgtttat |
| | | tactgtgcatcaaagttggaatatacttctaatgactatgactcctg |
| | | gggccaggggacccaggtcaccgtctcctca |
| 15 | single domain | QVQLQESGGGLVQAGGSLRLSCAASGRTVSSLFMGWFRQAP |
| | anti-MUC16 | GKERELVAAISRYSLYTYYADSVKGRFTISADNAKNAVYLQ |
| | binder R3MU4 | MNSLKPEDTAVYYCASKLEYTSNDYDSWGQGTQVTVSS |
| 16 | R3MU4CDR1 | GRTVSSLF |
| 17 | R3MU4 CDR2 | ISRYSLYT |
| 18 | R3MU4 CDR3 | ASKLEYTSNDYDS |
| 19 | Nucleic acid | caggtgcagctgcaggagtctgggggaggattggtgcaggctgggga |
| | sequence | ctctctgagactctcctgtgcagcctctggacgcgccgtcagtagct |
| | encoding | tgttcatgggctggttccgccgagctccagggaaggagcgtgaactt |
| | single domain | gtagcagccattagccggtatagtctatatacatactatgcagactc |
| | anti-MUC16 | cgtgaagggccgattcaccatctccgcagacaacgccaagaacgcgg |
| | R3MU29 | tatatctgcaaatgaacagcctaaaacctgaggacacggccgtttat |
| | | tactgtgcatcaaagttggaatatacttctaatgactatgactcctg |
| | | gggccaggggacccaggtcaccgtctcctca |
| 20 | Single domain | QVQLQESGGGLVQAGDSLRLSCAASGRAVSSLFMGWFRRAP |
| | anti-MUC16 | GKERELVAAISRYSLYTYYADSVKGRFTISADNAKNAVYLQ |
| | R3MU29 | MNSLKPEDTAVYYCASKLEYTSNDYDSWGQGTQVTVSS |
| 21 | R3MU29 CDR1 | GRAVSSLF |
| 22 | R3MU29 CDR2 | ISRYSLYT |
| 23 | R3MU29 CDR3 | ASKLEYTSNDYDS |
| 24 | Nucleic acid | caggtgcagctgcaggagtctgggggaggattggtgcaggctgggga |
| | sequence | ctctctgagactctcctgtgcagcctctggacgcaccgtcagtagct |
| | encoding | tgttcatggggtggttccgccgagctccagggaaggagcgtgaactt |
| | single domain | gtagcagccattagccggtatagtctatatacatactatgcagactc |
| | anti-MUC16 | cgtgaagggccgattcaccatctccgcagacaacgccaagaacgcgg |
| | R3MU63 | tatatctgcaaatgaacagcctgaaacctgaggacacggccgtttat |
| | | tactgtgcatcaaagttggaatatacttctaatgactatgactcctg |
| | | gggccaggggacccaggtcaccgtctcctca |
| 25 | Single domain | QVQLQESGGGLVQAGDSLRLSCAASGRTVSSLFMGWFRRAP |
| | anti-MUC16 | GKERELVAAISRYSLYTYYADSVKGRFTISADNAKNAVYLQ |
| | R3MU63 | MNSLKPEDTAVYYCASKLEYTSNDYDSWGQGTQVTVSS |
| 26 | R3MU63 CDR1 | GRTVSSLF |
| 27 | R3MU63 CDR2 | ISRYSLYT |
| 28 | R3MU63 CDR3 | ASKLEYTSNDYDS |
| 29 | Nucleic acid | caggtgcagctgcaggagtctgggggaggtttggtgcagcctgggga |
| | sequence | ttctatgagactctcctgtgcagccgagggggactctttggatggtt |
| | encoding | atgtagtaggttggttccgccaggccccagggaaggagcgccagggg |
| | single domain | gtctcaagtattagtggcgatggcagtatgcgatacgttgctgactc |
| | anti-MUC16 | cgtgaaggggcgattcaccatctcccgagacaacgccaagaacacgg |
| | R3MU119 | tgtatctgcaaatgatcgacctgaaacctgaggacacaggcgtttat |
| | | tactgtgcagcagacccacccacttgggactactggggtcaggggac |
| | | ccaggtcaccgtctcctca |
| 30 | Single domain | QVQLQESGGGLVQPGDSMRLSCAAEGDSLDGYVVGWFRQA |
| | anti-MUC16 | PGKERQGVSSISGDGSMRYVADSVKGRFTISRDNAKNTVYL |
| | R3MU119 | QMIDLKPEDTGVYYCAADPPTWDYWGQGTQVTVSS |
| 31 | R3MU119 | GDSLDGYV |
| | CDR1 | |
| 32 | R3MU119 | ISGDGSMR |
| | CDR2 | |
| 33 | R3MU119 | AADPPTWDY |
| | CDR3 | |
| 34 | Nucleic acid | caggtgcagctgcaggagtctgggggaggcttggtgcagcctggggg |
| | sequence | gtctctgagactctcctgtgcagcctctggacgcaccgtcagtagct |
| | encoding single | tgttcatgggctggttccgccgagctccagggaaggagcgtgaactt |
| | domain anti- | gtagcagccattagccggtatagtctatatacatactatgcagactc |
| | MUC16 | cgtgaagggccgattcaccatctccgcagacaacgccaagaacgcgg |
| | R3MU150 | tatatctgcaaatgaacagcctgaaacctgaggacacggccgtttat |
| | | tactgtgcatcaaagttggaatatacttctaatgactatgactcctg |
| | | gggccaggggacccaggtcaccgtctcctca |
| 35 | Single domain | QVQLQESGGGLVQPGGSLRLSCAASGRTVSSLFMGWFRRAP |
| | anti-MUC16 | GKERELVAAISRYSLYTYYADSVKGRFTISADNAKNAVYLQ |
| | R3MU150 | MNSLKPEDTAVYYCASKLEYTSNDYDSWGQGTQVTVSS |
| 36 | R3MU150 | GRTVSSLF |
| | CDR1 | |
| 37 | R3MU150 | ISRYSLYT |
| | CDR2 | |
| 38 | R3MU150 | ASKLEYTSNDYDS |
| | CDR3 | |
| 39 | Nucleic acid | caggtgcagctgcaggagtctgggggaggattggtgcaggctgggga |
| | sequence | gtctctgagactctcctgtgcagcctctggacgcaccgtcagtagct |
| | encoding single | tgttcatgggctggttccgccgagctccagggaaggagcgtgaactt |
| | domain anti- | gtagcagccattagccggtatagtctatatacatactatgcagactc |
| | MUC16 | cgtgaagggccgattcaccatctccgcagacaacgccaagaacgcgg |
| | R3MU147 | tatatctgcaaatgaacagcctgaaacctgaggacacggccgtttat |
| | | tactgtgcatcaaagttggaatatacttctaatgactatgactcctg |
| | | gggccaggggacccaggtcaccgtctcctca |
| 40 | Single domain | QVQLQESGGGLVQAGESLRLSCAASGRTVSSLFMGWFRRAP |
| | anti-MUC16 | GKERELVAAISRYSLYTYYADSVKGRFTISADNAKNAVYLQ |
| | R3MU147 | MNSLKPEDTAVYYCASKLEYTSNDYDSWGQGTQVTVSS |
| 41 | R3MU147 | GRTVSSLF |
| | CDR1 | |
| 42 | R3MU147 | ISRYSLYT |
| | CDR2 | |
| 43 | R3MU147 | ASKLEYTSNDYDS |
| | CDR3 | |
| 44 | R3MU29h15 | EVQLVESGGGLVQPGGSLRLSCAASGRAVSSLFMGWVRQAP |
| | (98.9% human) | GKGLEWVSAISRYSLYTYYADSVKGRFTISRDNAKNTLYLQ |
| | | MNSLRPEDTAVYYCASKLEYTSNDYDSWGQGTLVTVSS |
| 45 | R3MU29h14 | EVQLVESGGGLVQPGGSLRLSCAASGRAVSSLFMGWFRQAP |
| | (97.8% human) | GKGLEWVSAISRYSLYTYYADSVKGRFTISRDNAKNTLYLQ |
| | | MNSLRPEDTAVYYCASKLEYTSNDYDSWGQGTLVTVSS |
| 46 | R3MU29h13 | EVQLVESGGGLVQPGGSLRLSCAASGRAVSSLFMGWFRQAP |
| | (96.7% human) | GKGLELVSAISRYSLYTYYADSVKGRFTISRDNAKNTLYLQM |
| | | NSLRPEDTAVYYCASKLEYTSNDYDSWGQGTLVTVSS |
| 47 | R3MU4h13 | EVQLVESGGGLVQPGGSLRLSCAASGRTVSSLFMGWVRQAP |
| | (98.9% human | GKGLEWVSAISRYSLYTYYADSVKGRFTISRDNAKNTLYLQ |
| | | MNSLRPEDTAVYYCASKLEYTSNDYDSWGQGTLVTVSS |
| 48 | R3MU4h12 | EVQLVESGGGLVQPGGSLRLSCAASGRTVSSLFMGWFRQAP |
| | (97.8% human) | GKGLEWVSAISRYSLYTYYADSVKGRFTISRDNAKNTLYLQ |
| | | MNSLRPEDTAVYYCASKLEYTSNDYDSWGQGTLVTVSS |
| 49 | R3MU4h11 | EVQLVESGGGLVQPGGSLRLSCAASGRTVSSLFMGWFRQAP |
| | (96.7% human) | GKGLELVSAISRYSLYTYYADSVKGRFTISRDNAKNTLYLQM |
| | | NSLRPEDTAVYYCASKLEYTSNDYDSWGQGTLVTVSS |
| 50 | Nucleic acid | GATGTCCAGCTGGTGGAGTCTGGGGGAGGCTTGGTGCAGCCTGG |
| | sequence | GGGGTCTCTGAGACTCTCCTGTGCAGCCTCTGGATTCACTTCGGA |
| | encoding | TTATTATATCATAGGCTGGTTCCGCCAGGCCCCAGGGAAGGAGC |
| | Llama single | GCGAGGGGGTATCATGTATTAGTAGTAAATATGCGAACACAAAC |
| | domain anti- | TATGCAGACTCCGTGAAGGGCCGATTCACCCAGTCCAGAGGTGC |
| | IL13Rα2 Clone 1 | TGCTAAGAACACGGTGTATCTGCAAATGAACGCCCTGAAACCTG |
| | (LSD 1) | AGGACACGGCCGTTTATTACTGCGCGGCAGATACGAGGCGGTAT |
| | | ACATGCCCGGATATAGCGACTATGCACAGGAACTTTGATTCCTG |
| | | GGGCCAGGGGACCCAGGTCACCGTCTCCTCA |
| 51 | Llama single | DVQLVESGGGLVQPGGSLRLSCAASGFTSDYYIIGWFRQAPGKERE |
| | domain anti- | GVSCISSKYANTNYADSVKGRFTQSRGAAKNTVYLQMNALKPEDT |
| | IL13Rα2 Clone 1 | AVYYCAADTRRYTCPDIATMHRNFDSWGQGTQVTVSS |
| | (LSD1) | |
| 52 | LSD1 CDR1 | GFTSDYYI |
| 53 | LSD1 CDR2 | ISSKYANT |
| 54 | LSD1 CDR3 | AADTRRYTCPDIATMHRNFDS |
| 55 | Nucleic acid | GAaGTCCAGCTGGTGGAGTCTGGGGGAGGCTTGGTGCAGCCTGG |
| | sequence | GGGGTCTCTGAGACTCTCCTGTGCAGCCTCTGGATTCACTTCGGA |
| | encoding | TTATTATATCATgGGCTGGTTCCGCCAGGCCCCAGGGAAGGgcCtg |
| | Humanized Llama | GAGGGGGTATCATGTATTAGTAGTAAATATGCGAACACAtatTAT |
| | single domain | GCAGACTCCGTGAAGGGCCGATTCACCattTCCAGAGaTaacGCTA |
| | anti-IL13Rα2 | AGAACACGcTGTATCTGCAAATGAACagCCTGcgtCCTGAGGACAC |
| | Clone 1 | GGCCGTTTATTACTGCGCGGCAGATACGAGGCGGTATACATGCC |
| | (H1-LSD1) | CGGATATAGCGACTATGCACAGGAACTTTGATTCCTGGGGCCAG |
| | | GGGACCCtGGTCACCGTCTCCTCA |
| 56 | Humanized Llama | EVQLVESGGGLVQPGGSLRLSCAASGFTSDYYIMGWFRQAPGKGL |
| | Single domain | EGVSCISSKYANTYYADSVKGRFTISRDNAKNTLYLQMNSLRPEDT |
| | anti-IL13Rα2 | AVYYCAADTRRYTCPDIATMHRNFDSWGQGTLVTVSS |
| | Clone 1-h8 (H1- | |
| | LSD1) | |
| 57 | H1-LSD1 CDR1 | GFTSDYYI |
| 58 | H1-LSD1 CDR2 | ISSKYANT |
| 59 | H1-LSD1 CDR3 | AADTRRYTCPDIATMHRNFDS |
| 60 | Nucleic acid | GAaGTCCAGCTGGTGGAGTCTGGGGGAGGCTTGGTGCAGCCTGG |
| | sequence | GGGGTCTCTGAGACTCTCCTGTGCAGCCTCTGGATTCACTTCGGA |
| | encoding | TTATTATATCATgGGCTGGgtgCGCCAGGCCCCAGGGAAGGgcCtgG |
| | Humanized Llama | AGtGGGTATCATGTATTAGTAGTAAATATGCGAACACAtatTATGC |
| | single domain | AGACTCCGTGAAGGGCCGATTCACCattTCCAGAGaTaacGCTAAG |
| | anti-IL13Rα2 | AACACGcTGTATCTGCAAATGAACagCCTGcgtCCTGAGGACACGG |
| | Clone 1 | CCGTTTATTACTGCGCGGCAGATACGAGGCGGTATACATGCCCG |
| | (H2-LSD1) | GATATAGCGACTATGCACAGGAACTTTGATTCCTGGGGCCAGGG |
| | | GACCCtGGTCACCGTCTCCTCA |
| 61 | Humanized Llama | EVQLVESGGGLVQPGGSLRLSCAASGFTSDYYIMGWVRQAPGKGL |
| | single domain | EWVSCISSKYANTYYADSVKGRFTISRDNAKNTLYLQMNSLRPEDT |
| | anti-IL13Rα2 | AVYYCAADTRRYTCPDIATMHRNFDSWGQGTLVTVSS |
| | Clone 1-h10 | |
| | (H2-LSD1) | |
| 62 | H2-LSD1 CDR1 | GFTSDYYI |
| 63 | H2-LSD1 CDR2 | ISSKYANT |
| 64 | H2-LSD1 CDR3 | AADTRRYTCPDIATMHRNFDS |
| 65 | Nucleic acid | GATGTCCAGCTGGTGGAGTCTGGGGGAGGCTTGGTGCAGCCTGG |
| | sequence | GGGGTCTCTGAGACTCTCCTGTGAAGCCTCTGGATTCGCTTCGGA |
| | encoding | TGATTATATCATAGGCTGGTTCCGCCAGGCCCCAGGGAAGGAGC |
| | Llama single | GCGAGGGGGTTTCATGTATTAGTAGTAGGTATGCGAACACTGTC |
| | domain anti- | TATACAGACTCCGTGAAGGGCCGATTCCGCATCTCCAGAGGCAC |
| | IL13Rα2 Clone 2 | TGCTAAGAACACGGTGTATCTGCAAATGAGCGCCCTGAAACCTG |
| | (LSD2) | AGGACACGGCCGTTTATTACTGTGCGATGGATTCGAGGCGCGTT |
| | | ACATGCCCCGAGATATCGACTATGCACAGGAACTTTGATTCCTG |
| | | GGGCCAGGGGACCCAGGTCACCGTCTCCTCA |
| 66 | Llama single | DVQLVESGGGLVQPGGSLRLSCEASGFASDDYIIGWFRQAPGKERE |
| | domain anti- | GVSCISSRYANTVYTDSVKGRFRISRGTAKNTVYLQMSALKPEDTA |
| | IL13Ra2 Clone 2 | VYYCAMDSRRVTCPEISTMHRNFDSWGQGTQVTVSS |
| | (LSD2) | |
| 67 | LSD2 CDR1 | GFASDDYI |
| 68 | LSD2 CDR2 | ISSRYANT |
| 69 | LSD2 CDR3 | AMDSRRVTCPEISTMHRNFDS |
| 70 | Nucleic acid | GAaGTCCAGCTGGTGGAGTCTGGGGGAGGCTTGGTGCAGCCTGG |
| | sequence | GGGGTCTCTGAGACTCTCCTGTGcgGCCTCTGGATTCGCTTCGGA |
| | encoding | TGATTATATCATgGGCTGGTTCCGCCAGGCCCCAGGGAAGGgcCtg |
| | Humanized Llama | GAGGGGGTTTCATGTATTAGTAGTAGGTATGCGAACACTtatTATg |
| | single domain | CgGACTCCGTGAAGGGCCGATTCacCATCTCCAGAGatAacGCTAA |
| | anti-IL13Rα2 | GAACACGcTGTATCTGCAAATGAaCagCCTGcgtCCTGAGGACACG |
| | Clone 2 | GCCGTTTATTACTGTGCGATGGATTCGAGGCGCGTTACATGCCCC |
| | (H1-LSD2) | GAGATATCGACTATGCACAGGAACTTTGATTCCTGGGGCCAGGG |
| | | GACCCtGGTCACCGTCTCCTCA |
| 71 | Humanized Llama | EVQLVESGGGLVQPGGSLRLSCAASGFASDDYIMGWFRQAPGKGL |
| | Single domain | EGVSCISSRYANTYYADSVKGRFTISRDNAKNTLYLQMNSLRPEDT |
| | anti-IL13Rα2 | AVYYCAMDSRRVTCPEISTMHRNFDSWGQGTLVTVSS |
| | Clone 2-h8 | |
| | (H1-LSD2) | |
| 72 | Hl-LSD2 CDR1 | GFASDDYI |
| 73 | Hl-LSD2 CDR2 | ISSRYANT |
| 74 | Hl-LSD2 CDR3 | AMDSRRVTCPEISTMHRNFDS |
| 75 | Nucleic acid | GAaGTCCAGCTGGTGGAGTCTGGGGGAGGCTTGGTGCAGCCTGG |
| | sequence | GGGGTCTCTGAGACTCTCCTGTGcgGCCTCTGGATTCGCTTCGGA |
| | encoding | TGATTATATCATgGGCTGGgtgCGCCAGGCCCCAGGGAAGGgcCtgG |
| | Humanized Llama | AGtGGGTTTCATGTATTAGTAGTAGGTATGCGAACACTtatTATgCg |
| | single domain | GACTCCGTGAAGGGCCGATTCacCATCTCCAGAGatAacGCTAAGA |
| | anti-IL13Rα2 | ACACGcTGTATCTGCAAATGAaCagCCTGcgtCCTGAGGACACGGC |
| | Clone 2 | CGTTTATTACTGTGCGATGGATTCGAGGCGCGTTACATGCCCCGA |
| | (H2-LSD2) | GATATCGACTATGCACAGGAACTTTGATTCCTGGGGCCAGGGGA |
| | | CCCtGGTCACCGTCTCCTCA |
| 76 | Humanized Llama | EVQLVESGGGLVQPGGSLRLSCAASGFASDDYIMGWVRQAPGKGL |
| | single domain | EWVSCISSRYANTYYADSVKGRFTISRDNAKNTLYLQMNSLRPEDT |
| | anti-IL13Rα2 | AVYYCAMDSRRVTCPEISTMHRNFDSWGQGTLVTVSS |
| | Clone 2-h10 | |
| | (H2-LSD2) | |
| 77 | H2-LSD2 CDR1 | GFASDDYI |
| 78 | H2-LSD2 CDR2 | ISSRYANT |
| 79 | H2-LSD2 CDR3 | AMDSRRVTCPEISTMHRNFDS |
| 80 | 2TIG14 | QVQLQESGGGLVQAGGSLRLSCTASGLTFSTYS- |
| | | MGWFRQAPGKEREFVTALRWTGMDTWYADSVKGRFAISRDNAKN |
| | | TVYLQMNSLNAEDTAVYYCA-TRHKSVLG-- |
| | | AVANPTRYDYWGQGTQVTVSS |
| 81 | 2TIG23 | QVQLQESGGGLVQPGGSLRLSCTASGLTFSDYV- |
| | | MGWFRQAPGKEREFVARSTSTGY- |
| | | INYADPVKGRFTISRDDAKNTVYLQMNSLKPEDTAVYYCAATRY--- |
| | | -----VNRNREYDYWGQGTQVTVSS |
| 82 | 2TIG4 | QVQLQESGGGLVQPGGSLRLSCAASGRT---YG- |
| | | MGWFRQAPGKEREFVAVGVWSSGNTYYADFARGRFTISRDNAKN |
| | | TVYLQMDSLKPEDTAVYYCAAPRYSSY----- |
| | | TTYHAAYDYWGPGTQVTVSS |
| 83 | 2TIG52 | QVQLQESGGGLAQTGGSLRLSCDASARTFNKYV- |
| | | MGWFRQAPGKEREFVAAVNWDGDSTYYADDVKGRFTISRDNAKN |
| | | TVYLQMNSLKPEDTAVYYCAAWYGTT---- |
| | | WSPKVRNSYDYSGHGTQVTVSS |
| 84 | 2TIG21 | QVQLQESGGGLVQAGGSLRLSCTASGRTFSNYN- |
| | | LGWFRQAPGKEREFVAGVRWNYANTYYAESVKGRFKMSKDIAKN |
| | | TVYLQMNSLKPEDTAIYYCA------- |
| | | MGPKPGYELGPDDYWGQGTQVTVSS |
| 85 | 2TIG15 | QVQLQESGGGSVQAGGSLRLSCAPSGRSFS- |
| | | FRGMGWFRQAPGKEREFVAAASWIYATTDYSDSVKGRFTISKDNA |
| | | KDTLNLQMNSLKPEDTAVYYCAAVRGTSDT- |
| | | VLPPRSDYEYDVWGRGTQVTVSS |
| 86 | 2TIG35 | QVQLQESGGGLVQAGDSLRLSCAASGSTFSRYTNIGWFRQAPGKER |
| | | EFVAAFRWGFANTYYGDSVKGRSTISRDNAKKQVYLQMNSLKPED |
| | | TAVYYCAASSEW-------TTEAVKYDYWGQGTQVTVSS |
| 87 | 2TIG40 | QVQLQESGGGLVQAGGSLRLSCAASG--LSSNA- |
| | | MAWFRQGPGKDREFVAAFHWRFANTYYADSVKGRFTISRDNAKN |
| | | TVYLQMNSLKPEDTALYYCAARQGSVYGGSSPV---- |
| | | DYDYWGQGTQVTVSS |
| 88 | 2TIG6 | QVQLQESGGGLVQAGDSLKLSCVASGRTFSTYA- |
| | | MAWFRRAPGKEREFVASIIWSGGSSYYANSVKGRFTISGDNAKNTV |
| | | YLQMNGLKPEDTAVYYCAADNRPMGRS-TG------ |
| | | YNYWGQGTQVTVSS |
| 89 | 2TIG18 | QVQLQESGGGLVQAGGSLRLSCVDSGRTFGSYT- |
| | | MAWFRQAPGKEREFVAAISGSGGWKYYADSVKGRFTISRDNAKNT |
| | | VYLQMNSLKPEDTAVYYCA------- |
| | | GGLLPVTAAREYTYWGQGTQVTVSS |
| 90 | 2TIG54 | QVQLQESGGGLVQPGGSLRLSCAASGRTFSSY- |
| | | RMAWFRQAPGKESEFVAGIRWSGGRTYYADSVKGRFAISGDSAKN |
| | | MVYLQMNSLKSEDTAVYYCAADENSS------- |
| | | DQGYDYWGQGTQVTVSS |
| 91 | 2TIG66 | QVQLQESGGGLVQIGGSLRLSCAASGRTFSSY- |
| | | FMAWFRQAPGKEREFVAAIGWSGADTYYEDSVKGRFTISRDNANK |
| | | MVYLQMNSLKPEDTAVYYCASGRGS--------- |
| | | TWSTSTYSIRGQGTQVTVSS |
| 92 | 3TIG26 | QVQLQESGGGSVQAGGSLRLSCAASGRTFSDY- |
| | | YMAWFRQASGKEREFVATISRGGFNSDYADSAKGRFTISRDNAKN |
| | | TVYLQMNSLTPEDTAVYYCAADR----- |
| | | GIGDSRSATAYDYWGQGTQVTVSS |
| 93 | 3TIG35 | QVQLQESGGGLVQAGESLRLSCTASGLTDSNYA- |
| | | IGWFRQAPGKEREFVTESNWRGGNHYYLDSIKGRFTISRDNAKSTL |
| | | YLQMNNLQPEDTAVYYCAARR------------TARYDYWGQGTQVTVSS |
| 94 | 3TIG52 | QVQLQESGGGLVQAGASLKLSCAASGRTFSMYG- |
| | | LGWFRQAPGKEREFVASIRWSDNSTHYANSVKGRFTISADNAKNT |
| | | VYLQMNSLKPEDTAIYYCAG-----GRAGSP------ |
| | | LEYWGQGTQVTVSS |
| 95 | 3TIG53 | QVQLQESGGGSVQAGDSLRLSCAVSARTFSSYT- |
| | | MGWFRQAPGKEREFVTAITWSAGWTYYADSVKGRFTISRDNTQNT |
| | | VYLQMDSLKVEDTAVYYCAA------- |
| | | GPLPVTSPSSYDYWGQGTQVTVSS |
| 96 | Human MUC16 | NFSPLARRVDRVAIYEEFLRMTRNGTQLQNFTLDRSSVLVDGYSPN |
| | polypeptide | RNEPLTGNSDLP |
| 97 | Anti-MSLN | QVQLVQSGGGLVHPGGSLRLSCAASGIDLSLYRMRWYRQAPGKER |
| | VHH1 | DLVALITDDGTSYYEDSVKGRFTITRDNPSNKVFLQMNSLKPEDTA |
| | | VYYCNAETPLSPVNYWGQGTQVTVS |
| 98 | Anti-MSLN | QVQLVQSGGGLVQAGGSLRLSCAPSGSIFGIRTMDWYRQAPGKERE |
| | VHH2 | LVARITMDGRVFHADSVKGRFSGSRDGASNAVYLQMNSLKPDDTA |
| | | VYYCRYSGLTSREDYWGPGTQVTVSS |
| 99 | Short linker 3 | GGGGSGGGGS |
| 100 | DNA sequence of | GGTGGCGGAGGTTCTGGAGGTGGAGGTTCC |
| | short linker 3 | |
| 101 | MP057 primer | TTATGCTTCCGGCTCGTATG |
| 102 | Primer A6E | GATGTGCAGCTGCAGGAGTCTGGRGGAGG |
| 103 | Primer PMCF | CTAGTGCGGCCGCTGAGGAGACGGTGACCTGGGT |
| 104 | Universal | TCACACAGGAAACAGCTATGAC |
| | reverse primer | |
| 105 | Universal | CGCCAGGGTTTTCCCAGTCACGAC |
| | forward primer | |

1.-291. (canceled)

292. A pharmaceutical composition comprising
(I) a T cell from a human subject, wherein the T cell comprises a recombinant nucleic acid molecule encoding a T cell receptor (TCR) fusion protein (TFP) comprising
(a) a TCR subunit comprising
(i) at least a portion of a TCR extracellular domain,
(ii) a TCR transmembrane domain, and
(iii) a TCR intracellular domain; and
(b) an antigen binding domain comprising an anti-MUC16 binding domain; and
(II) a pharmaceutically acceptable carrier;
wherein the TCR subunit and the anti-MUC16 binding domain are operatively linked;
wherein the TFP functionally interacts with a TCR when expressed in the T cell.

293. The pharmaceutical composition of claim 292, wherein the TCR subunit and the anti-MUC16 binding domain are operatively linked by a linker.

294. The pharmaceutical composition of claim 293, wherein the linker comprises (G₄S)ₙ, wherein G is glycine, S is serine, and n is an integer from 1 to 4.

295. The pharmaceutical composition of claim 292, wherein the anti-MUC16 binding domain comprises
(i) a heavy chain (HC) CDR1 sequence GRTVSSLF, GRAVSSLF, or GDSLDGYV,
(ii) a HC CDR2 sequence ISRYSLYT, or ISGDGSMR, and
(iii) a HC CDR3 sequence ASKLEYTSNDYDS, or AADPPTWDY.

296. The pharmaceutical composition of claim 295, wherein the anti-MUC16 binding domain comprises a sequence having at least 70% sequence identity of SEQ ID NO: 15, SEQ ID NO:20, SEQ ID NO:25, SEQ ID NO:30, SEQ ID NO:35, or SEQ ID NO: 40.

297. The pharmaceutical composition of claim 292, wherein the pharmaceutical composition is substantially free of serum.

298. The pharmaceutical composition of claim 292, wherein the antigen binding domain is a scFv or a single domain antibody.

299. The pharmaceutical composition of claim 298, wherein the single domain antibody is a V_{H} domain.

300. The pharmaceutical composition of claim 292, wherein the T cell has greater than or more efficient cytotoxic activity than a T cell comprising a nucleic acid encoding a chimeric antigen receptor (CAR) comprising (a) the anti-MUC16 binding domain operatively linked to (b) at least a portion of a CD28 extracellular domain, (c) a CD28 transmembrane domain, (d) at least a portion of a CD28 intracellular domain and (e) a CD3 zeta intracellular domain.

301. The pharmaceutical composition of claim 292, wherein the TFP molecule functionally interacts with an endogenous TCR complex, at least one endogenous TCR polypeptide, or a combination thereof when expressed in the T cell.

302. The pharmaceutical composition of claim 292, wherein the T cell is a human CD4+ or a human CD8+ T cell.

303. The pharmaceutical composition of claim 292, wherein production of IL-2 or IFNγ by the T cell is increased in the presence of a cell expressing an antigen that specifically interacts with the anti-MUC16 binding domain compared to a T cell not containing the TFP.

304. The pharmaceutical composition of claim 292, wherein the TCR subunit is from a single subunit of a TCR complex, wherein the single subunit is CD3 epsilon, CD3 gamma or CD3 delta.

305. The pharmaceutical composition of claim 292, wherein the TCR transmembrane domain and the TCR intracellular domain of the TCR subunit are from a TCR alpha chain, a TCR beta chain, a TCR gamma chain, a TCR delta chain, CD3 epsilon, CD3 gamma, or CD3 delta.

306. The pharmaceutical composition of claim 292, wherein the TCR subunit is from a single subunit of a TCR complex, wherein the single subunit is a TCR alpha chain, a TCR beta chain, a TCR gamma chain or a TCR delta chain.

307. The pharmaceutical composition of claim 292, wherein the T cell exhibits increased cytotoxicity to a cell expressing an antigen that specifically interacts with the anti-MUC16 binding domain compared to a T cell not containing the TFP.

308. A method of treating cancer in a subject in need thereof comprising administering the pharmaceutical composition of claim 292 to the subject.

309. A recombinant nucleic acid comprising a sequence encoding a T cell receptor (TCR) fusion protein (TFP), wherein the TFP comprises:
(a) a TCR subunit comprising
(i) at least a portion of a TCR extracellular domain,
(ii) a TCR transmembrane domain, and
(iii) a TCR intracellular domain; and
(b) an antigen binding domain comprising an anti-MUC16 binding domain;
wherein the TCR subunit and the anti-MUC16 binding domain are operatively linked; and
wherein the TFP functionally interacts with a TCR when expressed in the T cell.

310. A pharmaceutical composition comprising
(I) a T cell from a human subject, wherein the T cell comprises a recombinant nucleic acid molecule encoding a T-cell receptor (TCR) fusion protein (TFP) comprising
(a) a TCR subunit comprising
(i) at least a portion of a TCR extracellular domain, and
(ii) a TCR transmembrane domain
(iii) a TCR intracellular domain; and
(b) an antibody domain comprising an anti-IL13Rα2 binding domain;
(II) a pharmaceutically acceptable cater;
wherein the TCR subunit and the anti-IL13Rα2 binding domain are operatively linked, and
wherein the TFP incorporates into a TCR when expressed in a T-cell.

311. The pharmaceutical composition of claim 310, wherein the TCR intracellular domain of the TCR subunit is from a TCR alpha chain, a TCR beta chain, a TCR gamma chain, a TCR delta chain, CD3 epsilon, CD3 gamma, or CD3 delta.


SEQUENCE LISTING (143 sequences)

SEQ ID NO: 1 - protein, 17 aa, Artificial Sequence
  Description of Artificial Sequence Synthetic peptide
GGGGSGGGGSGGGGSLE

SEQ ID NO: 2 - protein, 20 aa, Artificial Sequence
  Description of Artificial Sequence Synthetic peptide
AAAGGGGSGGGGSGGGGSLE

SEQ ID NO: 3 - protein, 30 aa, Artificial Sequence
  Description of Artificial Sequence Synthetic polypeptide
AAAIEVMYPPPYLGGGGSGGGGSGGGGSLE

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 6 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 281 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGAA

SEQ ID NO: 9 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 11 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 13 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 14 - DNA, 360 bp, Artificial Sequence
  Description of Artificial Sequence Synthetic polynucleotide
caggtgcagctgcaggagtctgggggaggattggtgcaggctgggggctctctgagactctcctgtgcagcctctggacgcaccgtcagtagcttgttcatgggctggttccgccaagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 15 - protein, 120 aa, Artificial Sequence
  Description of Artificial Sequence Synthetic polypeptide
QVQLQESGGGLVQAGGSLRLSCAASGRTVSSLFMGWFRQAPGKERELVAAISRYSLYTYYADSVKGRFTISADNAKNAVYLQMNSLKPEDTAVYYCASKLEYTSNDYDSWGQGTQVTVSS

SEQ ID NO: 16 - protein, 8 aa, Artificial Sequence
  Description of Artificial Sequence Synthetic peptide
GRTVSSLF

SEQ ID NO: 17 - protein, 8 aa, Artificial Sequence
  Description of Artificial Sequence Synthetic peptide
ISRYSLYT

SEQ ID NO: 18 - protein, 13 aa, Artificial Sequence
  Description of Artificial Sequence Synthetic peptide
ASKLEYTSNDYDS

SEQ ID NO: 19 - DNA, 360 bp, Artificial Sequence
  Description of Artificial Sequence Synthetic polynucleotide
caggtgcagctgcaggagtctgggggaggattggtgcaggctggggactctctgagactctcctgtgcagcctctggacgcgccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctaaaacctgaggacacggccgtttattactgtgcatcaaagttggaatatacttctaatgactatgactcctggggccaggggacccaggtcaccgtctcctca

SEQ ID NO: 20 - protein, 120 aa, Artificial Sequence
  Description of Artificial Sequence Synthetic polypeptide
QVQLQESGGGLVQAGDSLRLSCAASGRAVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 21 - protein, 8 aa, Artificial Sequence
  Description of Artificial Sequence Synthetic peptide
GRAVSSLF

SEQ ID NO: 22 - protein, 8 aa, Artificial Sequence
  Description of Artificial Sequence Synthetic peptide
ISRYSLYT

SEQ ID NO: 23 - protein, 13 aa, Artificial Sequence
  Description of Artificial Sequence Synthetic peptide
ASKLEYTSNDYDS

SEQ ID NO: 24 - DNA, 360 bp, Artificial Sequence
  Description of Artificial Sequence Synthetic polynucleotide
caggtgcagctgcaggagtctgggggaggattggtgcaggctggggactctctgagactctcctgtgcagcctctggacgcaccgtcagtagcttgttcatggggtggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 25 - protein, 120 aa, Artificial Sequence
  Description of Artificial Sequence Synthetic polypeptide
QVQLQESGGGLVQAGDSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 26 - protein, 8 aa, Artificial Sequence
  Description of Artificial Sequence Synthetic peptide
GRTVSSLF

SEQ ID NO: 27 - protein, 8 aa, Artificial Sequence
  Description of Artificial Sequence Synthetic peptide
ISRYSLYT

SEQ ID NO: 28 - protein, 13 aa, Artificial Sequence
  Description of Artificial Sequence Synthetic peptide
ASKLEYTSNDYDS

SEQ ID NO: 29 - DNA, 348 bp, Artificial Sequence
  Description of Artificial Sequence Synthetic polynucleotide
caggtgcagctgcaggagtctgggggaggtttggtgcagcctggggattctatgagactctcctgtgcagccgagggggactctttggatggttatgtagtaggttggttccgccaggccccagggaaggagcgccagggggtctcaagtattagtggcgatggcagtatgcgatacgttgctgactccgtgaaggggcgattcaccatctcccgagacaacgccaagaacacggtgtatctgcaaatgatcgacctgaaacctgaggacacaggcgtttattactgtgcagcagacccacccacttgggactactggggtcaggggacccaggtcaccgtctcctca

SEQ ID NO: 30 - protein, 116 aa, Artificial Sequence
  Description of Artificial Sequence Synthetic polypeptide
QVQLQESGGGLVQPGDSMRLSCAAEGDSLDGYVVGWFRQAPGKERQGVSSISGDGSMRYVADSVKGRFTISRDNAKNTVYLQMIDLKPEDTGVYYCAADPPTWDYWGQGTQVTVSS

SEQ ID NO: 31 - protein, 8 aa, Artificial Sequence
  Description of Artificial Sequence Synthetic peptide
GDSLDGYV

SEQ ID NO: 32 - protein, 8 aa, Artificial Sequence
  Description of Artificial Sequence Synthetic peptide
ISGDGSMR

SEQ ID NO: 33 - protein, 9 aa, Artificial Sequence
  Description of Artificial Sequence Synthetic peptide
AADPPTWDY

SEQ ID NO: 34 - DNA, 360 bp, Artificial Sequence
  Description of Artificial Sequence Synthetic polynucleotide
caggtgcagctgcaggagtctgggggaggcttggtgcagcctggggggtctctgagactctcctgtgcagcctctggacgcaccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 35 - protein, 120 aa, Artificial Sequence
  Description of Artificial Sequence Synthetic polypeptide
QVQLQESGGGLVQPGGSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 36 - protein, 8 aa, Artificial Sequence
  Description of Artificial Sequence Synthetic peptide
GRTVSSLF

SEQ ID NO: 37 - protein, 8 aa, Artificial Sequence
  Description of Artificial Sequence Synthetic peptide
ISRYSLYT

SEQ ID NO: 38 - protein, 13 aa, Artificial Sequence
  Description of Artificial Sequence Synthetic peptide
ASKLEYTSNDYDS

SEQ ID NO: 39 - DNA, 360 bp, Artificial Sequence
  Description of Artificial Sequence Synthetic polynucleotide
caggtgcagctgcaggagtctgggggaggattggtgcaggctggggagtctctgagactctcctgtgcagcctctggacgcaccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 40 - protein, 120 aa, Artificial Sequence
  Description of Artificial Sequence Synthetic polypeptide
QVQLQESGGGLVQAGESLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 41 - protein, 8 aa, Artificial Sequence
  Description of Artificial Sequence Synthetic peptide
GRTVSSLF

SEQ ID NO: 42 - protein, 8 aa, Artificial Sequence
  Description of Artificial Sequence Synthetic peptide
ISRYSLYT

SEQ ID NO: 43 - protein, 13 aa, Artificial Sequence
  Description of Artificial Sequence Synthetic peptide
ASKLEYTSNDYDS

SEQ ID NO: 44 - protein, 120 aa, Artificial Sequence
  Description of Artificial Sequence Synthetic polypeptide
EVQLVESGGGLVQPGGSLRLSCAASGRAVSSLFMGWVRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 45 - protein, 120 aa, Artificial Sequence
  Description of Artificial Sequence Synthetic polypeptide
EVQLVESGGGLVQPGGSLRLSCAASGRAVSSLFMGWFRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 46 - protein, 120 aa, Artificial Sequence
  Description of Artificial Sequence Synthetic polypeptide
EVQLVESGGGLVQPGGSLRLSCAASGRAVSSLFMGWFRQAPGKGLELVSAISRYSLYTYYADSVKGRFTISRDNAKNTLYLQMNSLRPEDTAVYYCASKLEYTSNDYDSWGQGTLVTVSS

SEQ ID NO: 47 - protein, 120 aa, Artificial Sequence
  Description of Artificial Sequence Synthetic polypeptide
EVQLVESGGGLVQPGGSLRLSCAASGRTVSSLFMGWVRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 48 - protein, 120 aa, Artificial Sequence
  Description of Artificial Sequence Synthetic polypeptide
EVQLVESGGGLVQPGGSLRLSCAASGRTVSSLFMGWFRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 49 - protein, 120 aa, Artificial Sequence
  Description of Artificial Sequence Synthetic polypeptide
EVQLVESGGGLVQPGGSLRLSCAASGRTVSSLFMGWFRQAPGKGLELVSAISRYSLYTYYADSVKGRFTISRDNAKNTLYLQMNSLRPEDTAVYYCASKLEYTSNDYDSWGQGTLVTVSS

SEQ ID NO: 50 - DNA, 384 bp, Artificial Sequence
  Description of Artificial Sequence Synthetic polynucleotide
gatgtccagctggtggagtctgggggaggcttggtgcagcctggggggtctctgagactctcctgtgcagcctctggattcacttcggattattatatcataggctggttccgccaggccccagggaaggagcgcgagggggtatcatgtattagtagtaaatatgcgaacacaaactatgcagactccgtgaagggccgattcacccagtccagaggtgctgctaagaacacggtgtatctgcaaatgaacgccctgaaacctgaggacacggccgtttattactgcgcggcagatacgaggcggtatacatgcccggatatagcgactatgcacaggaactttgattcctggggccaggggacccaggtcaccgtctcctca

SEQ ID NO: 51 - protein, 128 aa, Artificial Sequence
  Description of Artificial Sequence Synthetic polypeptide
DVQLVESGGGLVQPGGSLRLSCAASGFTSDYYIIGWFRQAPGKEREGVSCISSKYANTNYADSVKGRFTQSRGAAKNTVYLQMNALKPEDTAVYYCAADTRRYTCPDIATMHRNFDSWGQGTQVTVSS

SEQ ID NO: 52 - protein, 8 aa, Artificial Sequence
  Description of Artificial Sequence Synthetic peptide
GFTSDYYI

SEQ ID NO: 53 - protein, 8 aa, Artificial Sequence
  Description of Artificial Sequence Synthetic peptide
ISSKYANT

SEQ ID NO: 54 - protein, 21 aa, Artificial Sequence
  Description of Artificial Sequence Synthetic peptide
AADTRRYTCPDIATMHRNFDS

SEQ ID NO: 55 - DNA, 384 bp, Artificial Sequence
  Description of Artificial Sequence Synthetic polynucleotide
gaagtccagctggtggagtctgggggaggcttggtgcagcctggggggtctctgagactctcctgtgcagcctctggattcacttcggattattatatcatgggctggttccgccaggccccagggaagggcctggagggggtatcatgtattagtagtaaatatgcgaacacatattatgcagactccgtgaagggccgattcaccatttccagagataacgctaagaacacgctgtatctgcaaatgaacagcctgcgtcctgaggacacggccgtttattactgcgcggcagatacgaggcggtatacatgcccggatatagcgactatgcacaggaactttgattcctggggccaggggaccctggtcaccgtctcctca

SEQ ID NO: 56 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTYYADSVKGRFTISRDNAKNTLYLQMNSLRPEDTAVYYCAADTRRYTCPDIATMHRNFDSWGQGTLVTVSS

SEQ ID NO: 57 - protein, 8 aa, Artificial Sequence
  Description of Artificial Sequence Synthetic peptide
GFTSDYYI

SEQ ID NO: 58 - protein, 8 aa, Artificial Sequence
  Description of Artificial Sequence Synthetic peptide
ISSKYANT

SEQ ID NO: 59 - protein, 21 aa, Artificial Sequence
  Description of Artificial Sequence Synthetic peptide
AADTRRYTCPDIATMHRNFDS

SEQ ID NO: 60 - DNA, 384 bp, Artificial Sequence
  Description of Artificial Sequence Synthetic polynucleotide
gaagtccagctggtggagtctgggggaggcttggtgcagcctggggggtctctgagactctcctgtgcagcctctggattcacttcggattattatatcatgggctgggtgcgccaggccccagggaagggcctggagtgggtatcatgtattagtagtaaatatgcgaacacatattatgcagactccgtgaagggccgattcaccatttccagagataacgctaagaacacgctgtatctgcaaatgaacagcctgcgtcctgaggacacggccgtttattactgcgcggcagatacgaggcggtatacatgcccggatatagcgactatgcacaggaactttgattcctggggccaggggaccctggtcaccgtctcctca

SEQ ID NO: 61 - protein, 128 aa, Artificial Sequence
  Description of Artificial Sequence Synthetic polypeptide
EVQLVESGGGLVQPGGSLRLSCAASGFTSDYYIMGWVRQAPGKGLEWVSCISSKYANTYYADSVKGRFTISRDNAKNTLYLQMNSLRPEDTAVYYCAADTRRYTCPDIATMHRNFDSWGQGTLVTVSS

SEQ ID NO: 62 - protein, 8 aa, Artificial Sequence
  Description of Artificial Sequence Synthetic peptide
GFTSDYYI

SEQ ID NO: 63 - protein, 8 aa, Artificial Sequence
  Description of Artificial Sequence Synthetic peptide
ISSKYANT

SEQ ID NO: 64 - protein, 21 aa, Artificial Sequence
  Description of Artificial Sequence Synthetic peptide
AADTRRYTCPDIATMHRNFDS

SEQ ID NO: 65 - DNA, 384 bp, Artificial Sequence
  Description of Artificial Sequence Synthetic polynucleotide
gatgtccagctggtggagtctgggggaggcttggtgcagcctggggggtctctgagactctcctgtgaagcctctggattcgcttcggatgattatatcataggctggttccgccaggccccagggaaggagcgcgagggggtttcatgtattagtagtaggtatgcgaacactgtctatacagactccgtgaagggccgattccgcatctccagaggcactgctaagaacacggtgtatctgcaaatgagcgccctgaaacctgaggacacggccgtttattactgtgcgatggattcgaggcgcgttacatgccccgagatatcgactatgcacaggaactttgattcctggggccaggggacccaggtcaccgtctcctca

SEQ ID NO: 66 - protein, 128 aa, Artificial Sequence
  Description of Artificial Sequence Synthetic polypeptide
DVQLVESGGGLVQPGGSLRLSCEASGFASDDYIIGWFRQAPGKEREGVSCISSRYANTVYTDSVKGRFRISRGTAKNTVYLQMSALKPEDTAVYYCAMDSRRVTCPEISTMHRNFDSWGQGTQVTVSS

SEQ ID NO: 67 - protein, 8 aa, Artificial Sequence
  Description of Artificial Sequence Synthetic peptide
GFASDDYI

SEQ ID NO: 68 - protein, 8 aa, Artificial Sequence
  Description of Artificial Sequence Synthetic peptide
ISSRYANT

SEQ ID NO: 69 - protein, 21 aa, Artificial Sequence
  Description of Artificial Sequence Synthetic peptide
AMDSRRVTCPEISTMHRNFDS

SEQ ID NO: 70 - DNA, 384 bp, Artificial Sequence
  Description of Artificial Sequence Synthetic polynucleotide
gaagtccagctggtggagtctgggggaggcttggtgcagcctggggggtctctgagactctcctgtgcggcctctggattcgcttcggatgattatatcatgggctggttccgccaggccccagggaagggcctggagggggtttcatgtattagtagtaggtatgcgaacacttattatgcggactccgtgaagggccgattcaccatctccagagataacgctaagaacacgctgtatctgcaaatgaacagcctgcgtcctgaggacacggccgtttattactgtgcgatggattcgaggcgcgttacatgccccgagatatcgactatgcacaggaactttgattcctggggccaggggaccctggtcaccgtctcctca

SEQ ID NO: 71 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTYYADSVKGRFTISRDNAKNTLYLQMNSLRPEDTAVYYCAMDSRRVTCPEISTMHRNFDSWGQGTLVTVSS

SEQ ID NO: 72 - protein, 8 aa, Artificial Sequence
  Description of Artificial Sequence Synthetic peptide
GFASDDYI

SEQ ID NO: 73 - protein, 8 aa, Artificial Sequence
  Description of Artificial Sequence Synthetic peptide
ISSRYANT

SEQ ID NO: 74 - protein, 21 aa, Artificial Sequence
  Description of Artificial Sequence Synthetic peptide
AMDSRRVTCPEISTMHRNFDS

SEQ ID NO: 75 - DNA, 384 bp, Artificial Sequence
  Description of Artificial Sequence Synthetic polynucleotide
gaagtccagctggtggagtctgggggaggcttggtgcagcctggggggtctctgagactctcctgtgcggcctctggattcgcttcggatgattatatcatgggctgggtgcgccaggccccagggaagggcctggagtgggtttcatgtattagtagtaggtatgcgaacacttattatgcggactccgtgaagggccgattcaccatctccagagataacgctaagaacacgctgtatctgcaaatgaacagcctgcgtcctgaggacacggccgtttattactgtgcgatggattcgaggcgcgttacatgccccgagatatcgactatgcacaggaactttgattcctggggccaggggaccctggtcaccgtctcctca

SEQ ID NO: 76 - protein, 128 aa, Artificial Sequence
  Description of Artificial Sequence Synthetic polypeptide
EVQLVESGGGLVQPGGSLRLSCAASGFASDDYIMGWVRQAPGKGLEWVSCISSRYANTYYADSVKGRFTISRDNAKNTLYLQMNSLRPEDTAVYYCAMDSRRVTCPEISTMHRNFDSWGQGTLVTVSS

SEQ ID NO: 77 - protein, 8 aa, Artificial Sequence
  Description of Artificial Sequence Synthetic peptide
GFASDDYI

SEQ ID NO: 78 - protein, 8 aa, Artificial Sequence
  Description of Artificial Sequence Synthetic peptide
ISSRYANT

SEQ ID NO: 79 - protein, 21 aa, Artificial Sequence
  Description of Artificial Sequence Synthetic peptide
AMDSRRVTCPEISTMHRNFDS

SEQ ID NO: 80 - protein, 126 aa, Artificial Sequence
  Description of Artificial Sequence Synthetic polypeptide
QVQLQESGGGLVQAGGSLRLSCTASGLTFSTYSMGWFRQAPGKEREFVTALRWTGMDTWYADSVKGRFAISRDNAKNTVYLQMNSLNAEDTAVYYCATRHKSVLGAVANPTRYDYWGQGTQVTVSS

SEQ ID NO: 81 - protein, 120 aa, Artificial Sequence
  Description of Artificial Sequence Synthetic polypeptide
QVQLQESGGGLVQPGGSLRLSCTASGLTFSDYVMGWFRQAPGKEREFVARSTSTGYINYADPVKGRFTISRDDAKNTVYLQMNSLKPEDTAVYYCAATRYVNRNREYDYWGQGTQVTVSS

SEQ ID NO: 82 - protein, 121 aa, Artificial Sequence
  Description of Artificial Sequence Synthetic polypeptide
QVQLQESGGGLVQPGGSLRLSCAASGRTYGMGWFRQAPGKEREFVAVGVWSSGNTYYADFARGRFTISRDNAKNTVYLQMDSLKPEDTAVYYCAAPRYSSYTTYHAAYDYWGPGTQVTVSS

SEQ ID NO: 83 - protein, 125 aa, Artificial Sequence
  Description of Artificial Sequence Synthetic polypeptide
QVQLQESGGGLAQTGGSLRLSCDASARTFNKYVMGWFRQAPGKEREFVAAVNWDGDSTYYADDVKGRFTISRDNAKNTVYLQMNSLKPEDTAVYYCAAWYGTTWSPKVRNSYDYSGHGTQVTVSS

SEQ ID NO: 84 - protein, 122 aa, Artificial Sequence
  Description of Artificial Sequence Synthetic polypeptide
QVQLQESGGGLVQAGGSLRLSCTASGRTFSNYNLGWFRQAPGKEREFVAGVRWNYANTYYAESVKGRFKMSKDIAKNTVYLQMNSLKPEDTAIYYCAMGPKPGYELGPDDYWGQGTQVTVSS

SEQ ID NO: 85 - protein, 128 aa, Artificial Sequence
  Description of Artificial Sequence Synthetic polypeptide
QVQLQESGGGSVQAGGSLRLSCAPSGRSFSFRGMGWFRQAPGKEREFVAAASWIYATTDYSDSVKGRFTISKDNAKDTLNLQMNSLKPEDTAVYYCAAVRGTSDTVLPPRSDYEYDVWGRGTQVTVSS

SEQ ID NO: 86 - protein, 123 aa, Artificial Sequence
  Description of Artificial Sequence Synthetic polypeptide
QVQLQESGGGLVQAGDSLRLSCAASGSTFSRYTNIGWFRQAPGKEREFVAAFRWGFANTYYGDSVKGRSTISRDNAKKQVYLQMNSLKPEDTAVYYCAASSEWTTEAVKYDYWGQGTQVTVSS

SEQ ID NO: 87 - protein, 123 aa, Artificial Sequence
  Description of Artificial Sequence Synthetic polypeptide
QVQLQESGGGLVQAGGSLRLSCAASGLSSNAMAWFRQGPGKDREFVAAFHWRFANTYYADSVKGRFTISRDNAKNTVYLQMNSLKPEDTALYYCAARQGSVYGGSSPVDYDYWGQGTQVTVSS

SEQ ID NO: 88 - protein, 122 aa, Artificial Sequence
  Description of Artificial Sequence Synthetic polypeptide
QVQLQESGGGLVQAGDSLKLSCVASGRTFSTYAMAWFRRAPGKEREFVASIIWSGGSSYYANSVKGRFTISGDNAKNTVYLQMNGLKPEDTAVYYCAADNRPMGRSTGYNYWGQGTQVTVSS

SEQ ID NO: 89 - protein, 122 aa, Artificial Sequence
  Description of Artificial Sequence Synthetic polypeptide
QVQLQESGGGLVQAGGSLRLSCVDSGRTFGSYTMAWFRQAPGKEREFVAAISGSGGWKYYADSVKGRFTISRDNAKNTVYLQMNSLKPEDTAVYYCAGGLLPVTAAREYTYWGQGTQVTVSS

SEQ ID NO: 90 - protein, 120 aa, Artificial Sequence
  Description of Artificial Sequence Synthetic polypeptide
QVQLQESGGGLVQPGGSLRLSCAASGRTFSSYRMAWFRQAPGKESEFVAGIRWSGGRTYYADSVKGRFAISGDSAKNMVYLQMNSLKSEDTAVYYCAADENSSDQGYDYWGQGTQVTVSS

SEQ ID NO: 91 - protein, 122 aa, Artificial Sequence
  Description of Artificial Sequence Synthetic polypeptide
QVQLQESGGGLVQIGGSLRLSCAASGRTFSSYFMAWFRQAPGKEREFVAAIGWSGADTYYEDSVKGRFTISRDNANKMVYLQMNSLKPEDTAVYYCASGRGSTWSTSTYSIRGQGTQVTVSS

SEQ ID NO: 92 - protein, 124 aa, Artificial Sequence
  Description of Artificial Sequence Synthetic polypeptide
QVQLQESGGGSVQAGGSLRLSCAASGRTFSDYYMAWFRQASGKEREFVATISRGGFNSDYADSAKGRFTISRDNAKNTVYLQMNSLTPEDTAVYYCAADRGIGDSRSATAYDYWGQGTQVTVSS

SEQ ID NO: 93 - protein, 117 aa, Artificial Sequence
  Description of Artificial Sequence Synthetic polypeptide
QVQLQESGGGLVQAGESLRLSCTASGLTDSNYAIGWFRQAPGKEREFVTESNWRGGNHYYLDSIKGRFTISRDNAKSTLYLQMNNLQPEDTAVYYCAARRTARYDYWGQGTQVTVSS

SEQ ID NO: 94 - protein, 118 aa, Artificial Sequence
  Description of Artificial Sequence Synthetic polypeptide
QVQLQESGGGLVQAGASLKLSCAASGRTFSMYGLGWFRQAPGKEREFVASIRWSDNSTHYANSVKGRFTISADNAKNTVYLQMNSLKPEDTAIYYCAGGRAGSPLEYWGQGTQVTVSS

SEQ ID NO: 95 - protein, 122 aa, Artificial Sequence
  Description of Artificial Sequence Synthetic polypeptide
QVQLQESGGGSVQAGDSLRLSCAVSARTFSSYTMGWFRQAPGKEREFVTAITWSAGWTYYADSVKGRFTISRDNTQNTVYLQMDSLKVEDTAVYYCAAGPLPVTSPSSYDYWGQGTQVTVSS

SEQ ID NO: 96 - protein, 58 aa, Homo sapiens
NFSPLARRVDRVAIYEEFLRMTRNGTQLQNFTLDRSSVLVDGYSPNRNEPLTGNSDLP

SEQ ID NO: 97 - protein, 116 aa, Artificial Sequence
  Description of Artificial Sequence Synthetic polypeptide
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 98 - protein, 117 aa, Artificial Sequence
  Description of Artificial Sequence Synthetic polypeptide
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 99 - protein, 10 aa, Artificial Sequence
  Description of Artificial Sequence Synthetic peptide
GGGGSGGGGS

SEQ ID NO: 100 - DNA, 30 bp, Artificial Sequence
  Description of Artificial Sequence Synthetic oligonucleotide
ggtggcggaggttctggaggtggaggttcc

SEQ ID NO: 101 - DNA, 20 bp, Artificial Sequence
  Description of Artificial Sequence Synthetic oligonucleotide
ttatgcttccggctcgtatg

SEQ ID NO: 102 - DNA, 29 bp, Artificial Sequence
  Description of Artificial Sequence Synthetic oligonucleotide
gatgtgcagctgcaggagtctggrggagg

SEQ ID NO: 103 - DNA, 34 bp, Artificial Sequence
  Description of Artificial Sequence Synthetic oligonucleotide
ctagtgcggccgctgaggagacggtgacctgggt

SEQ ID NO: 104 - DNA, 22 bp, Artificial Sequence
  Description of Artificial Sequence Synthetic oligonucleotide
tcacacaggaaacagctatgac

SEQ ID NO: 105 - DNA, 24 bp, Artificial Sequence
  Description of Artificial Sequence Synthetic oligonucleotide
cgccagggttttcccagtcacgac

SEQ ID NO: 106 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 107 - protein, 20 aa, Artificial Sequence
  Description of Artificial Sequence Synthetic peptide
  MISC_FEATURE (1)..(20): This sequence may encompass 1-4 “Gly Gly Gly Gly Ser” repeating units
GGGGSGGGGSGGGGSGGGGS

SEQ ID NO: 108 - protein, 20 aa, Artificial Sequence
  Description of Artificial Sequence Synthetic peptide
  MISC_FEATURE (1)..(20): This sequence may encompass 2-4 “Gly Gly Gly Gly Ser” repeating units
GGGGSGGGGSGGGGSGGGGS

SEQ ID NO: 109 - protein, 15 aa, Artificial Sequence
  Description of Artificial Sequence Synthetic peptide
  MISC_FEATURE (1)..(15): This sequence may encompass 1-3 “Gly Gly Gly Gly Ser” repeating units
GGGGSGGGGSGGGGS

SEQ ID NO: 110 - protein, 4 aa, Artificial Sequence
  Description of Artificial Sequence Synthetic peptide
  See specification as filed for detailed description of substitutions and preferred embodiments
GGGS

SEQ ID NO: 111 - protein, 20 aa, Artificial Sequence
  Description of Artificial Sequence Synthetic peptide
GGGGSGGGGSGGGGSGGGGS

SEQ ID NO: 112 - protein, 15 aa, Artificial Sequence
  Description of Artificial Sequence Synthetic peptide
GGGGSGGGGSGGGGS

SEQ ID NO: 113 - protein, 4 aa, Artificial Sequence
  Description of Artificial Sequence Synthetic peptide
GGGS

SEQ ID NO: 114 - DNA, 5000 bp, Artificial Sequence
  Description of Artificial Sequence Synthetic polynucleotide
  misc_feature (1)..(5000): This sequence may encompass 50-5000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 115 - protein, 30 aa, Artificial Sequence
  Description of Artificial Sequence Synthetic polypeptide
  MISC_FEATURE (1)..(30): This sequence may encompass 1-6 “Gly Gly Gly Gly Ser” repeating units
  See specification as filed for detailed description of substitutions and preferred embodiments
GGGGSGGGGSGGGGSGGGGSGGGGSGGGGS

SEQ ID NO: 116 - protein, 5 aa, Artificial Sequence
  Description of Artificial Sequence Synthetic peptide
  See specification as filed for detailed description of substitutions and preferred embodiments
GGGGS

SEQ ID NO: 117 - DNA, 2000 bp, Artificial Sequence
  Description of Artificial Sequence Synthetic polynucleotide
  misc_feature (1)..(2000): This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - DNA, 100 bp, Artificial Sequence
  Description of Artificial Sequence Synthetic polynucleotide
tttttttttttttttttttttttttttttttttttttttttttttttttttttttttttttttttttttttttttttttttttttttttttttttttttt

SEQ ID NO: 119 - DNA, 5000 bp, Artificial Sequence
  Description of Artificial Sequence Synthetic polynucleotide
  misc_feature (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 120 - DNA, 5000 bp, Artificial Sequence; identical to SEQ ID NO: 114
  Description of Artificial Sequence Synthetic polynucleotide
  misc_feature (1)..(5000): This sequence may encompass 100-5000 nucleotides

SEQ ID NO: 121 - DNA, 400 bp, Artificial Sequence
  Description of Artificial Sequence Synthetic polynucleotide
  misc_feature (1)..(400): This sequence may encompass 100-400 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - protein, 5 aa, Artificial Sequence
  Description of Artificial Sequence Synthetic peptide
GGGGS

SEQ ID NO: 123 - protein, 10 aa, Artificial Sequence
  Description of Artificial Sequence Synthetic peptide
GGGGSGGGGS

SEQ ID NO: 124 - protein, 6 aa, Artificial Sequence
  Description of Artificial Sequence Synthetic 6xHis tag
HHHHHH

SEQ ID NO: 125 - protein, 58 aa, Artificial Sequence; identical to SEQ ID NO: 96
  Description of Artificial Sequence Synthetic polypeptide

SEQ ID NO: 126 - protein, 58 aa, Artificial Sequence; identical to SEQ ID NO: 96
  Description of Artificial Sequence Synthetic polypeptide

SEQ ID NO: 127 - protein, 58 aa, Artificial Sequence; identical to SEQ ID NO: 96
  Description of Artificial Sequence Synthetic polypeptide

SEQ ID NO: 128 - protein, 128 aa, Artificial Sequence
  Description of Artificial Sequence Synthetic polypeptide
EVQLVESGGGLVQPGGSLRLSCAASGFTSDYYIMGWFRQAPGKGLEWVSCISSKYANTYYADSVKGRFTISRDNAKNTLYLQMNSLRPEDTAVYYCAADTRRYTCPDIATMHRNFDSWGQGTLVTVSS

SEQ ID NO: 129 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTYYADSVKGRFTISRDNAKNTLYLQMNSLRPEDTAVYYCAADTRRYTCPDIATMHRNFDSWGQGTQVTVSS

SEQ ID NO: 130 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTNYADSVKGRFTISRDNAKNTLYLQMNSLRPEDTAVYYCAADTRRYTCPDIATMHRNFDSWGQGTQVTVSS

SEQ ID NO: 131 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTNYADSVKGRFTISRGNAKNTLYLQMNSLRPEDTAVYYCAADTRRYTCPDIATMHRNFDSWGQGTQVTVSS

SEQ ID NO: 132 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTNYADSVKGRFTISRGNAKNTLYLQMNALRPEDTAVYYCAADTRRYTCPDIATMHRNFDSWGQGTQVTVSS

SEQ ID NO: 133 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTNYADSVKGRFTISRGNAKNTVYLQMNALRPEDTAVYYCAADTRRYTCPDIATMHRNFDSWGQGTQVTVSS

SEQ ID NO: 134 - protein, 128 aa, Artificial Sequence
  Description of Artificial Sequence Synthetic polypeptide
EVQLVESGGGLVQPGGSLRLSCAASGFTSDYYIMGWFRQAPGKGLEGVSCISSKYANTNYADSVKGRFTQSRGNAKNTVYLQMNALRPEDTAVYYCAADTRRYTCPDIATMHRNFDSWGQGTQVTVSS

SEQ ID NO: 135 - protein, 128 aa, Artificial Sequence
  Description of Artificial Sequence Synthetic polypeptide
EVQLVESGGGLVQPGGSLRLSCAASGFTSDYYIMGWFRQAPGKELEGVSCISSKYANTNYADSVKGRFTQSRGNAKNTVYLQMNALRPEDTAVYYCAADTRRYTCPDIATMHRNFDSWGQGTQVTVSS

SEQ ID NO: 136 - protein, 128 aa, Artificial Sequence
  Description of Artificial Sequence Synthetic polypeptide
EVQLVESGGGLVQPGGSLRLSCAASGFASDDYIMGWFRQAPGKGLEWVSCISSRYANTYYADSVKGRFTISRDNAKNTLYLQMNSLRPEDTAVYYCAMDSRRVTCPEISTMHRNFDSWGQGTLVTVSS

SEQ ID NO: 137 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTYYADSVKGRFTISRDNAKNTLYLQMNSLRPEDTAVYYCAMDSRRVTCPEISTMHRNFDSWGQGTQVTVSS

SEQ ID NO: 138 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFTISRDNAKNTLYLQMNSLRPEDTAVYYCAMDSRRVTCPEISTMHRNFDSWGQGTQVTVSS

SEQ ID NO: 139 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFTISRDNAKNTVYLQMNSLRPEDTAVYYCAMDSRRVTCPEISTMHRNFDSWGQGTQVTVSS

SEQ ID NO: 140 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFTISRGNAKNTVYLQMNSLRPEDTAVYYCAMDSRRVTCPEISTMHRNFDSWGQGTQVTVSS

SEQ ID NO: 141 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFTISRGNAKNTVYLQMSSLRPEDTAVYYCAMDSRRVTCPEISTMHRNFDSWGQGTQVTVSS

SEQ ID NO: 142 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFTISRGNAKNTVYLQMSALRPEDTAVYYCAMDSRRVTCPEISTMHRNFDSWGQGTQVTVSS

SEQ ID NO: 143 - protein, 128 aa, Artificial Sequence
  Description of Artificial Sequence Synthetic polypeptide
EVQLVESGGGLVQPGGSLRLSCAASGFASDDYIMGWFRQAPGKGLEGVSCISSRYANTVYADSVKGRFRISRGNAKNTVYLQMSALRPEDTAVYYCAMDSRRVTCPEISTMHRNFDSWGQGTQVTVSS